(19) United States
(12) Patent Application Publication
(10) Pub. No.: US 2002/0172968 A1
(43) Pub. Date: Nov. 21, 2002

(54) Biochips comprising nucleic acid/protein conjugates

(76) Inventors: Hongxiang Liu, Monrovia, CA (US); Bassil I. Dahiyat, Altadena, CA (US); Min Li, Lutherville, MD (US)

Correspondence Address: Robin M. Silva, FLEHR HOHBACH TEST ALBRITTON & HERBERT LLP, Suite 3400, Four Embarcadero Center, San Francisco, CA, 94111-4187, US

(21) Appl. No.: 10/080,376

(22) Filed: Feb. 19, 2002

Related U.S. Application Data
(63) Continuation-in-part of application No. 09/792,630, filed on Feb. 22, 2001.

(51) Int. Cl. C12Q 1/68; C07H 21/04

(52) U.S. Cl. 435/6; 536/24.3

(57) ABSTRACT

The present invention is directed to the formation of protein arrays through the use of nucleic acid/protein (NAP) conjugates, which allow the covalent attachment of proteins and the nucleic acids encoding them. By using vectors that include capture sequences that will hybridize to capture probes on a nucleic acid array, the NAP conjugates including the proteins of interest are arrayed and used in a wide variety of applications.

This application is a continuation of U.S. Ser. No. 09/792,630, filed Feb. 22, 2001.

### FIELD OF THE INVENTION

The present invention is directed to the formation of protein arrays through the use of nucleic acid/protein (NAP) conjugates, which allow the covalent attachment of proteins and the nucleic acids encoding them. By using vectors that include capture sequences that will hybridize to capture probes on a nucleic acid array, the NAP conjugates including the proteins of interest are arrayed and used in a wide variety of applications.

### BACKGROUND OF THE INVENTION

There are a wide variety of known nucleic acid array technologies, which utilize immobilized capture probes on a wide variety of surfaces, for the detection and/or quantification of nucleic acids. These surfaces can comprise any number of different substrates, including silicon, glass, electrodes, plastics, etc.

These biochips are used in a wide variety of different assays, including diagnostic applications, gene expression profiling, and mutation detection (often referred to as single nucleotide polymorphism (SNP) detection when single base substitutions are at issue).

However, while nucleic acid biochips are useful in a large number of applications, there is an increasing awareness that an evaluation of a cell's protein content and variety is increasingly important. That is, an evaluation of the genetic content of a cell (whether genomic or mRNA, or both) provides a great deal of knowledge; however, the proteins of a cell that are expressed at any particular time (sometimes referred to in the art as the proteome of the cell) is becoming an increasing important and lucrative area.

Thus, it is an object of the present invention to provide methods of arraying proteins for use in a wide variety of applications.

### SUMMARY OF THE INVENTION

In accordance with the objects outlined above, the present invention provides compositions comprising a substrate comprising an array of capture probes, a plurality of which are hybridized to a nucleic acid/protein (NAP) conjugate. The NAP conjugates comprise a fusion polypeptide comprising a nucleic acid modification (NAM) enzyme and a candidate protein, and an expression vector comprising an enzyme attachment sequence (EAS), a capture sequence and a fusion nucleic acid. The fusion nucleic acid comprises a nucleic acid encoding a NAM enzyme and a nucleic acid encoding a candidate protein, wherein the EAS and the NAM enzyme are covalently attached. In a preferred embodiment, the NAM enzyme is a Rep protein.

In a further aspect, the nucleic acid sequence encoding the candidate protein is derived from cDNA, genomic DNA or a random peptide.

In an additional aspect, the invention provides methods of detecting the presence of a target analyte in a sample comprising contacting the sample with a biochip as outlined above and detecting the presence of the target analyte on the substrate.

### BRIEF DESCRIPTION OF THE DRAWINGS

FIG. 1 (SEQ ID NO:1) depicts the amino acid sequence of Rep78 isolated from adeno-associated virus 2.

FIG. 2 (SEQ ID NO:2) depicts the nucleotide sequence of Rep78 isolated from adeno-associated virus 2.

FIG. 3 (SEQ ID NO:3) depicts the amino acid sequence of major coat protein A isolated from adeno-associated virus 2.

FIG. 4 (SEQ ID NO:4) depicts the nucleotide sequence of major coat protein A isolated from adeno-associated virus 2.

FIG. 5 (SEQ ID NO:5) depicts the amino acid sequence of a Rep protein isolated from adeno-associated virus 4.

FIG. 6 (SEQ ID NO:6) depicts the nucleotide sequence of a Rep protein isolated from adeno-associated virus 4.

FIG. 7 (SEQ ID NO:7) depicts the amino acid sequence of Rep78 isolated from adeno-associated virus 3B.

FIG. 8 (SEQ ID NO:8) depicts the nucleotide sequence of Rep78 isolated from adeno-associated virus 3B.

FIG. 9 (SEQ ID NO:9) depicts the amino acid sequence of a nonstructural protein isolated from adeno-associated virus 3.

FIG. 10 (SEQ ID NO:10) depicts the nucleotide sequence of a nonstructural protein isolated from adeno-associated virus 3.

FIG. 11 (SEQ ID NO:11) depicts the amino acid sequence of a nonstructural protein isolated from adeno-associated virus 1.

FIG. 12 (SEQ ID NO:12) depicts the nucleotide sequence of a nonstructural protein isolated from adeno-associated virus 1.

FIG. 13 (SEQ ID NO:13) depicts the amino acid sequence of Rep78 isolated from adeno-associated virus6.

FIG. 14 (SEQ ID NO:14) depicts the nucleotide sequence of Rep78 isolated from adeno-associated virus 6.

FIG. 15 (SEQ ID NO:15) depicts the amino acid sequence of Rep68 isolated from adeno-associated virus 2.

FIG. 16 (SEQ ID NO:16) depicts the nucleotide sequence of Rep68 isolated from adeno-associated virus 2.

FIG. 17 (SEQ ID NO:17) depicts the amino acid sequence of major coat protein A′ (alt.) isolated from adeno-associated virus 2.

FIG. 18 (SEQ ID NO:18) depicts the nucleotide sequence of major coat protein A′ (alt.) isolated from adeno-associated virus 2.

FIG. 19 (SEQ ID NO:19) depicts the amino acid sequence of major coat protein A″ (alt.) isolated from adeno-associated virus 2.

FIG. 20 (SEQ ID NO:20) depicts the nucleotide sequence of major coat protein A″ (alt.) isolated from adeno-associated virus 2.

FIG. 21 (SEQ ID NO:21) depicts the amino acid sequence of a Rep protein isolated from adeno-associated virus 5.

FIG. 22 (SEQ ID NO:22) depicts the nucleotide sequence of a Rep protein isolated from adeno-associated virus 5.

FIG. 23 (SEQ ID NO:23) depicts the amino acid sequence of major coat protein Aa (alt.) isolated from adeno-associated virus 2.

FIG. 24 (SEQ ID NO:24) depicts the nucleotide sequence of major coat protein Aa (alt.) isolated from adeno-associated virus 2.

FIG. 25 (SEQ ID NO:25) depicts the amino acid sequence of a Rep protein isolated from Barbarie duck parvovirus.

FIG. 26 (SEQ ID NO:26) depicts the nucleotide sequence of a Rep protein isolated from Barbarie duck parvovirus.

FIG. 27 (SEQ ID NO:27) depicts the amino acid sequence of a Rep protein isolated from goose parvovirus.

FIG. 28 (SEQ ID NO:28) depicts the nucleotide sequence of a Rep protein isolated from goose parvovirus.

FIG. 29 (SEQ ID NO:29) depicts the amino acid sequence of NS1 protein isolated from muscovy duck parvovirus.

FIG. 30 (SEQ ID NO:30) depicts the nucleotide sequence of NS1 protein isolated from muscovy duck parvovirus.

FIG. 31 (SEQ ID NO:31) depicts the amino acid sequence of NS1 protein isolated from goose parvovirus.

FIG. 32 (SEQ ID NO:32) depicts the nucleotide sequence of NS1 protein isolated from goose parvovirus.

FIG. 33 (SEQ ID NO:33) depicts the amino acid sequence of a nonstructural protein isolated from chipmunk parvovirus.

FIG. 34 (SEQ ID NO:34) depicts the nucleotide sequence of a nonstructural protein isolated from chipmunk parvovirus.

FIG. 35 (SEQ ID NO:35) depicts the amino acid sequence of a nonstructural protein isolated from the pig-tailed macaque parvovirus.

FIG. 36 (SEQ ID NO:36) depicts the nucleotide sequence of a nonstructural protein isolated from the pig-tailed macaque parvovirus.

FIG. 37 (SEQ ID NO:37) depicts the amino acid sequence of NS1 protein isolated from a simian parvovirus.

FIG. 38 (SEQ ID NO:38) depicts the nucleotide sequence of NS1 protein isolated from a simian parvovirus.

FIG. 39 (SEQ ID NO:39) depicts the amino acid sequence of a NS protein isolated from the Rhesus macaque parvovirus.

FIG. 40 (SEQ ID NO:40) depicts the nucleotide sequence of a NS protein isolated from the Rhesus macaque parvovirus.

FIG. 41 (SEQ ID NO:41) depicts the amino acid sequence of a nonstructural protein isolated from the B19 virus.

FIG. 42 (SEQ ID NO:42) depicts the nucleotide sequence of a nonstructural protein isolated from the B19 virus.

FIG. 43 (SEQ ID NO:43) depicts the amino acid sequence of orf1 isolated from the Erythrovirus B19.

FIG. 44 (SEQ ID NO:44) depicts the nucleotide sequence of orf1 isolated from the Erythrovirus B19.

FIG. 45 (SEQ ID NO:45) depicts the amino acid sequence of U94 isolated from the human herpesvirus 6B.

FIG. 46 (SEQ ID NO:46) depicts the nucleotide sequence of U94 isolated from the human herpesvirus 6B.

FIG. 47 (SEQ ID NO:47) depicts an enzyme attachment sequence for a Rep protein.

FIG. 48 (SEQ ID NO:48) depicts the Rep68 and Rep78 enzyme attachment site found in chromosome 19.

FIGS. 49A-49N depict preferred embodiments of the expression vectors of the invention.

FIG. 50 depicts the synthesis of a full-length gene and all possible mutations by PCR. Overlapping oligonucleotides corresponding to the full-length gene (black bar, Step 1) are synthesized, heated and annealed. Addition of Pfu DNA polymerase to the annealed oligonucleotides results in the 5′-3′ synthesis of DNA (Step 2) to produce longer DNA fragments (Step 3). Repeated cycles of heating, annealing (Step 4) results in the production of longer DNA, including some full-length molecules. These can be selected by a second round of PCR using primers (arrowed) corresponding to the end of the full-length gene (Step 5).

FIG. 51 depicts the reduction of the dimensionality of sequence space by PDA screening. From left to right, 1: without PDA; 2: without PDA not counting Cysteine, Proline, Glycine; 3: with PDA using the 1% criterion, modeling free enzyme; 4: with PDA using the 1% criterion, modeling enzyme-substrate complex; 5: with PDA using the 5% criterion modeling free enzyme; 6: with PDA using the 5% criterion modeling enzyme-substrate complex.

FIG. 52 depicts a preferred scheme for synthesizing a library of the invention. The wild-type gene, or any starting gene, such as the gene for the global minima gene, can be used. Oligonucleotides comprising different amino acids at the different variant positions can be used during PCR using standard primers. This generally requires fewer oligonucleotides and can result in fewer errors.

FIG. 53 depicts and overlapping extension method. At the top of FIG. 53 is the template DNA showing the locations of the regions to be mutated (black boxes) and the binding sites of the relevant primers (arrows). The primers R1 and R2 represent a pool of primers, each containing a different mutation; as described herein, this may be done using different ratios of primers if desired. The variant position is flanked by regions of homology sufficient to get hybridization. In this example, three separate PCR reactions are done for step 1. The first reaction contains the template plus oligos F1 and R1. The second reaction contains template plus F2 and R2, and the third contains the template and F3 and R3. The reaction products are shown. In Step 2, the products from Step 1 tube 1 and Step 1 tube 2 are taken. After purification away from the primers, these are added to a fresh PCR reaction together with F1 and R4. During the Denaturation phase of the PCR, the overlapping regions anneal and the second strand is synthesized. The product is then amplified by the outside primers. In Step 3, the purified product from Step 2 is used in a third PCR reaction, together with the product of Step 1, tube 3 and the primers F1 and R3. The final product corresponds to the full length gene and contains the required mutations.

FIG. 54 depicts a ligation of PCR reaction products to synthesize the libraries of the invention. In this technique, the primers also contain an endonuclease restriction site (RE), either blunt, 5′ overhanging or 3′ overhanging. We set up three separate PCR reactions for Step 1. The first reaction contains the template plus oligos F1 and R1. The second reaction contains template plus F2 and R2, and the third contains the template and F3 and R3. The reaction products are shown. In Step 2, the products of step 1 are purified and then digested with the appropriate restriction endonuclease. The digestion products from Step 2, tube 1 and Step 2, tube 2 and ligate them together with DNA ligase (step 3). The products are then amplified in Step 4 using primer F1 and R4. The whole process is then repeated by digesting the amplified products, ligating them to the digested products of Step 2, tube 3, and then amplifying the final product by primers F1 and R3. It would also be possible to ligate all three PCR products from Step 1 together in one reaction, providing the two restriction sites (RE1 and RE2) were different.

FIG. 55 depicts blunt end ligation of PCR products. In this technique, the primers such as F1 and R1 do not overlap, but they abut. Again three separate PCR reactions are performed. The products from tube 1 and tube 2 are ligated, and then amplified with outside primers F1 and R4. This product is then ligated with the product from Step 1, tube 3. The final products are then amplified with primers F1 and R3.

FIG. 56 depicts M13 single stranded template production of mutated PCR products. Primer1 and Primer2 (each representing a pool of primers corresponding to desired mutations) are mixed with the M13 template containing the wildtype gene or any starting gene. PCR produces the desired product (11) containing the combinations of the desired mutations incorporated in Primer1 and Primer2. This scheme can bp used to produce a gene with mutations, or fragments of a gene with mutations that are then linked together via ligation or PCR for example.

FIG. 57 depicts preferred biochip configurations. FIG. 57A depicts a nucleic acid biochip in which a capture probe/capture sequence hybridization complex is used to form an array of NAM expression vectors. FIG. 57B depicts a protein biochip in which a capture probe/capture sequence hybridization complex is used to form an array of NAM expression vectors/NAP conjugates. FIG. 57C depicts a bifunctional chip in which some pads contain capture probe/capture sequence hybridization complexes hybridized to NAM expression vectors and other pads contain capture probe/capture sequence hybridization complexes hybridized to NAM expression vectors/NAP conjugates. FIG. 57D depicts an in vitro expression method for making a protein chip in which the NAP conjugates are covalently linked to the EAS sequence of the expression vector. FIG. 57E depicts an in vitro expression method for making a protein chip in which the NAP conjugates are not covalently attached to the EAS sequence of the expression vector.

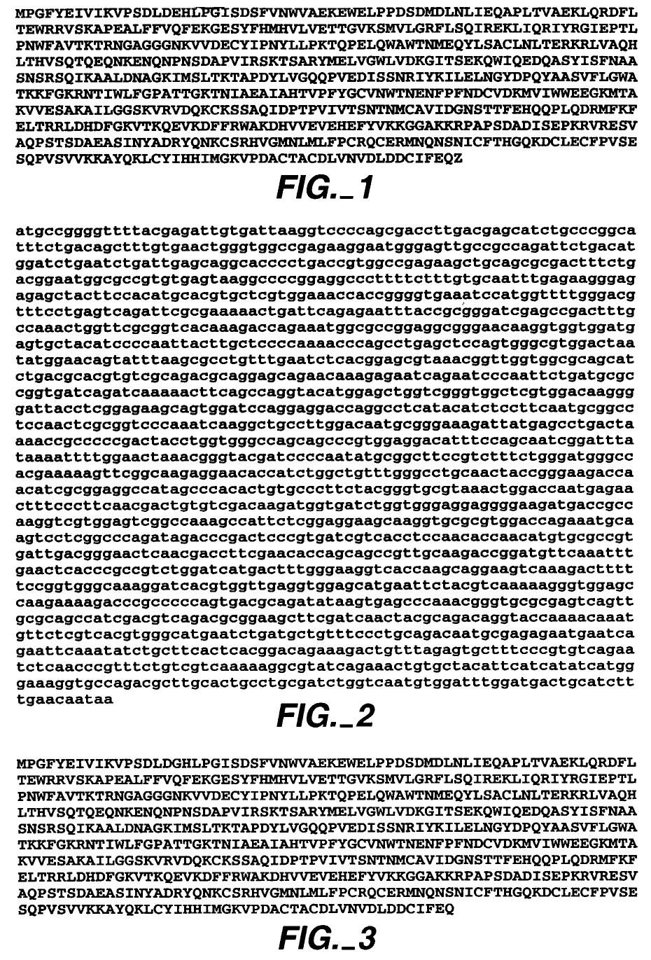

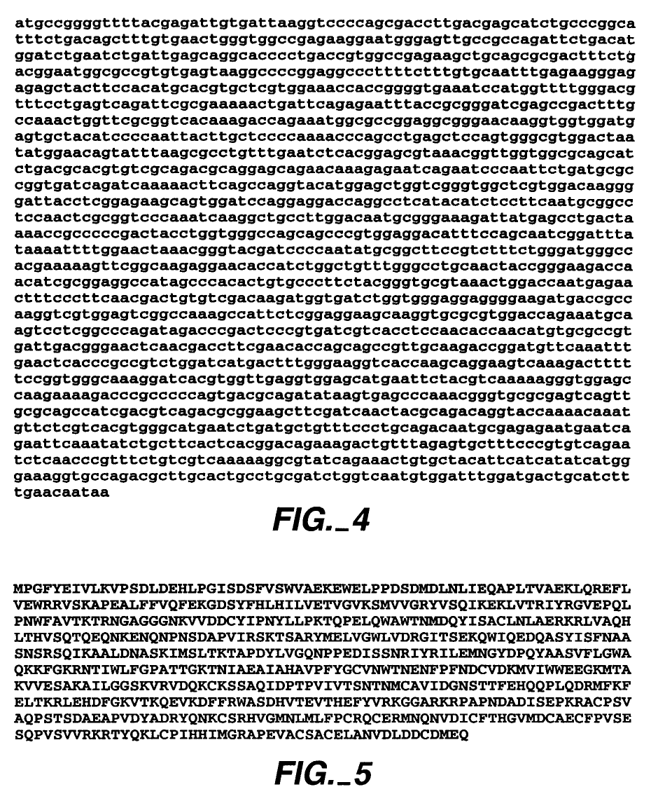

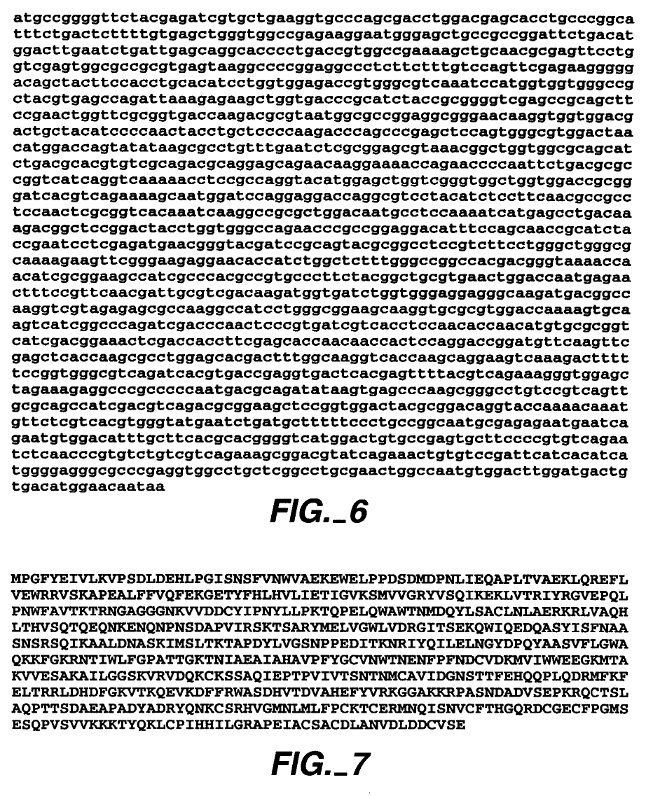

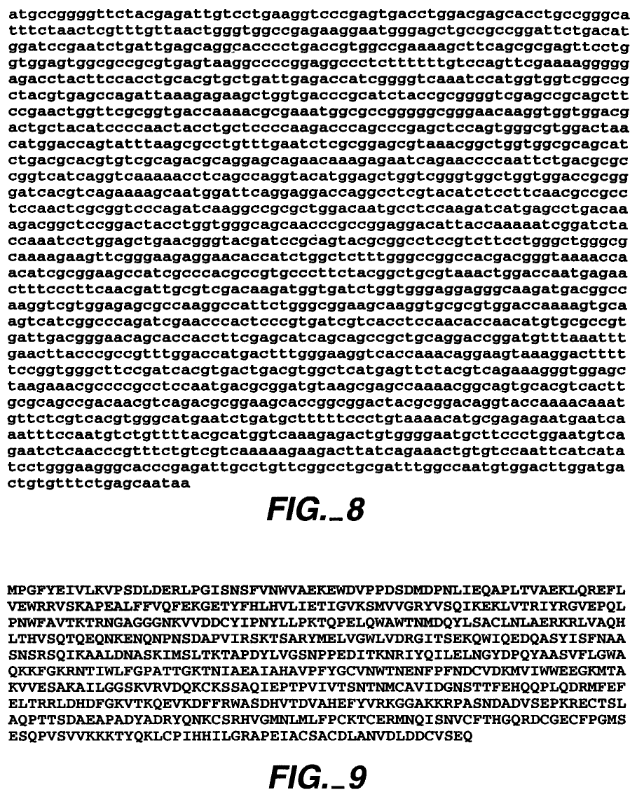

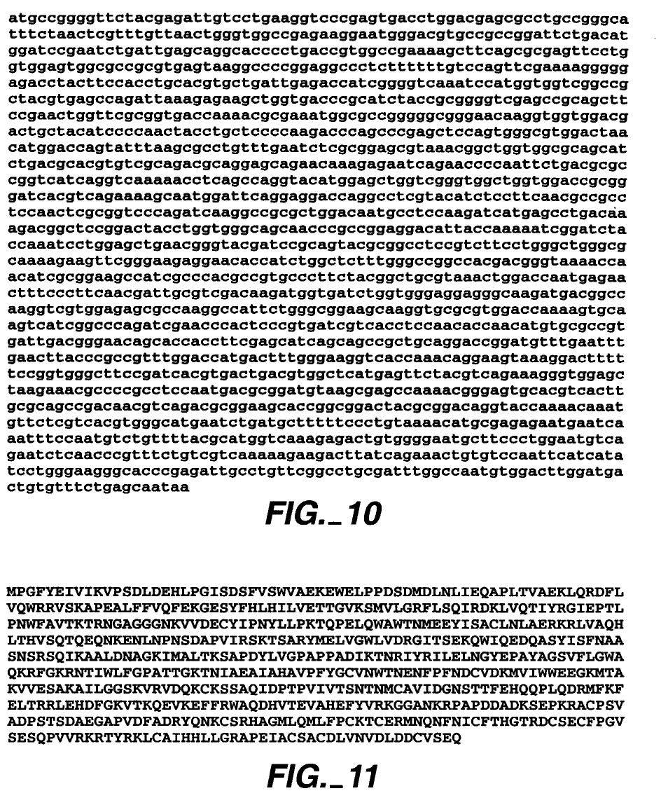

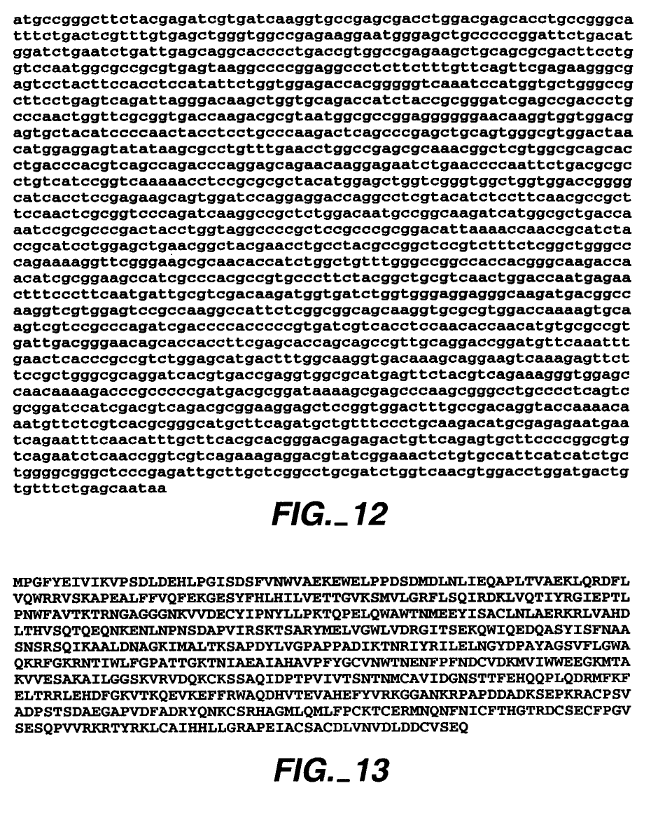

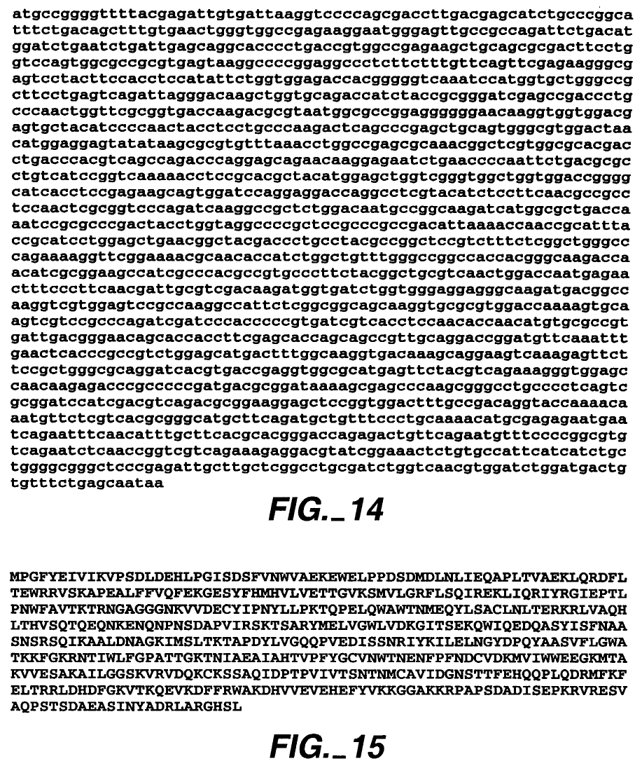

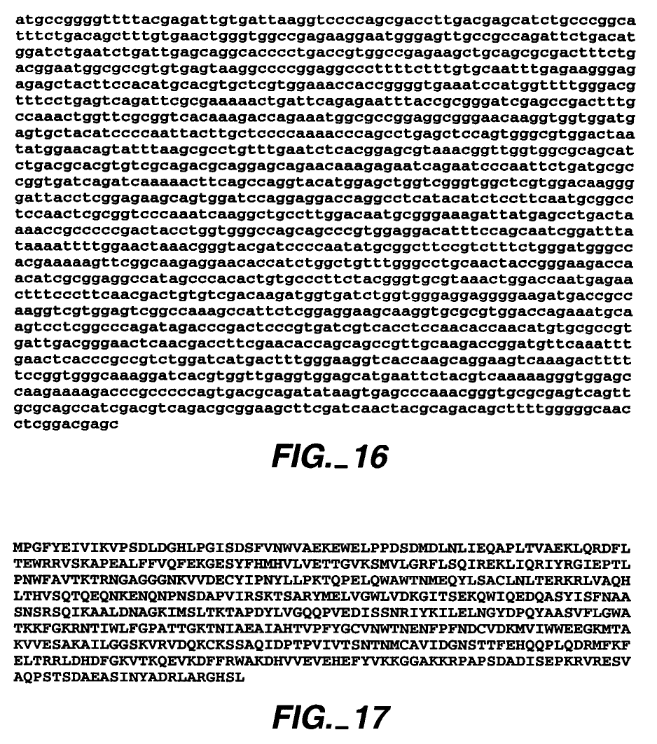

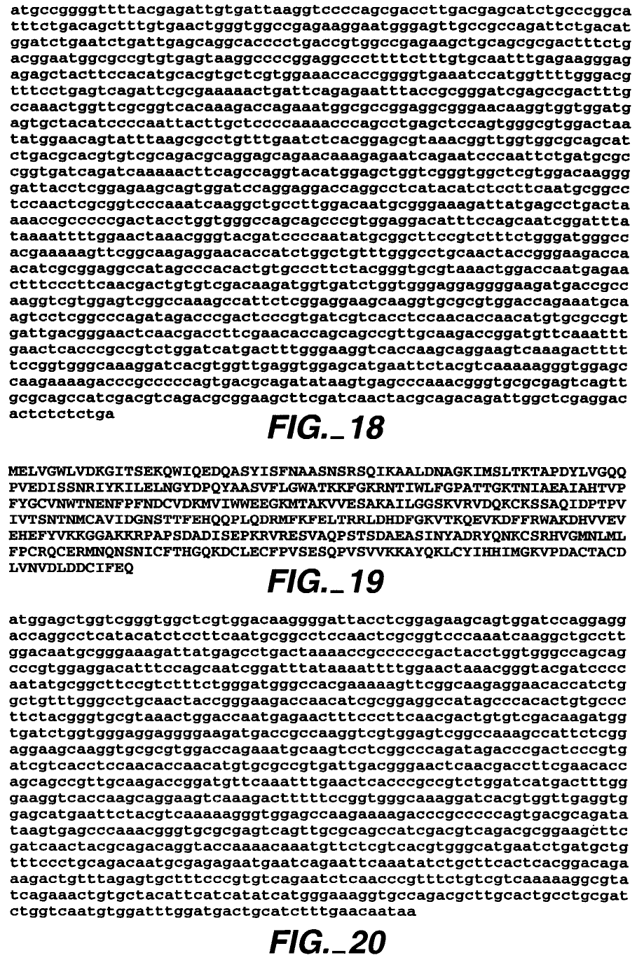

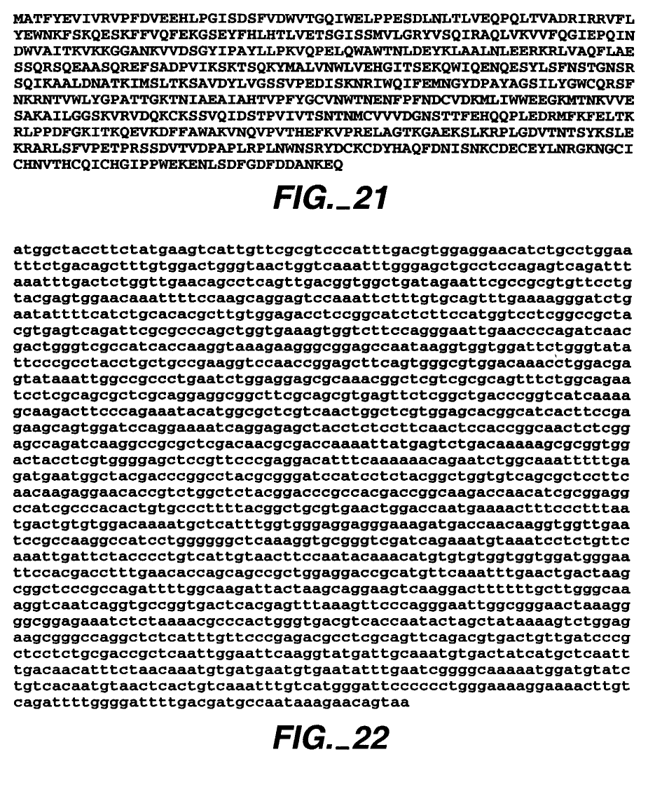

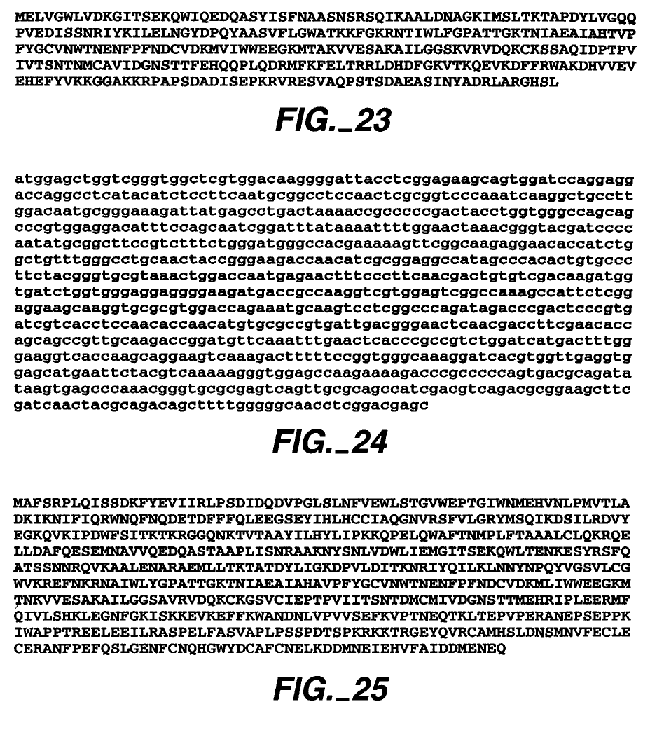

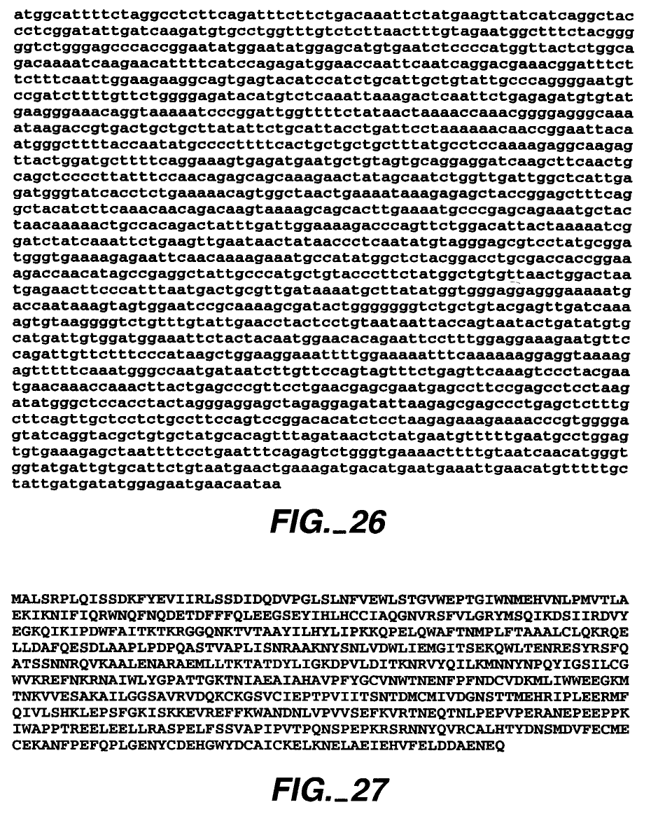

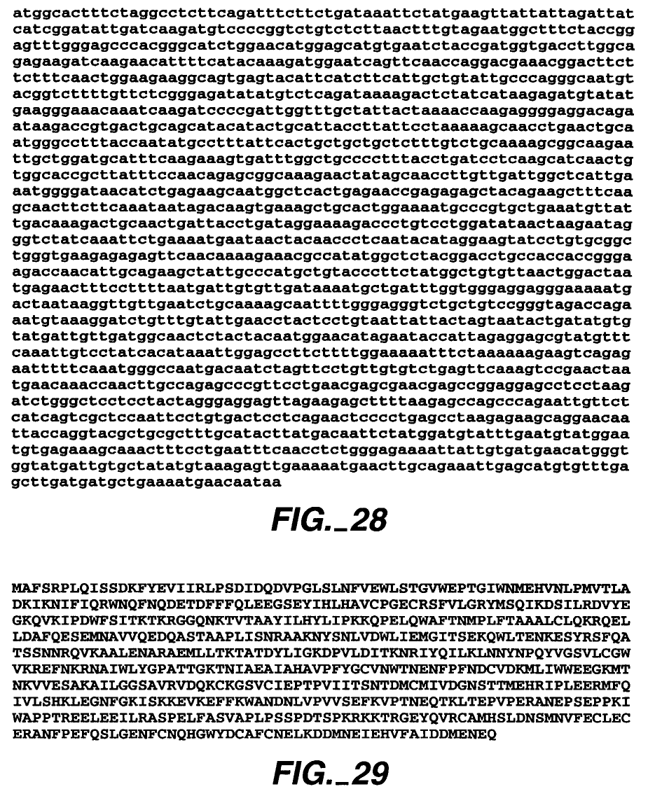

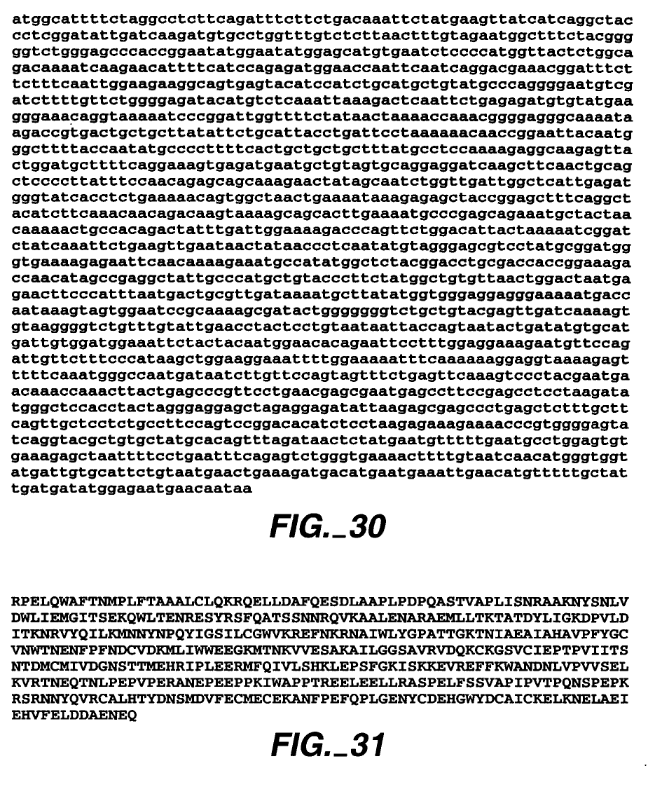

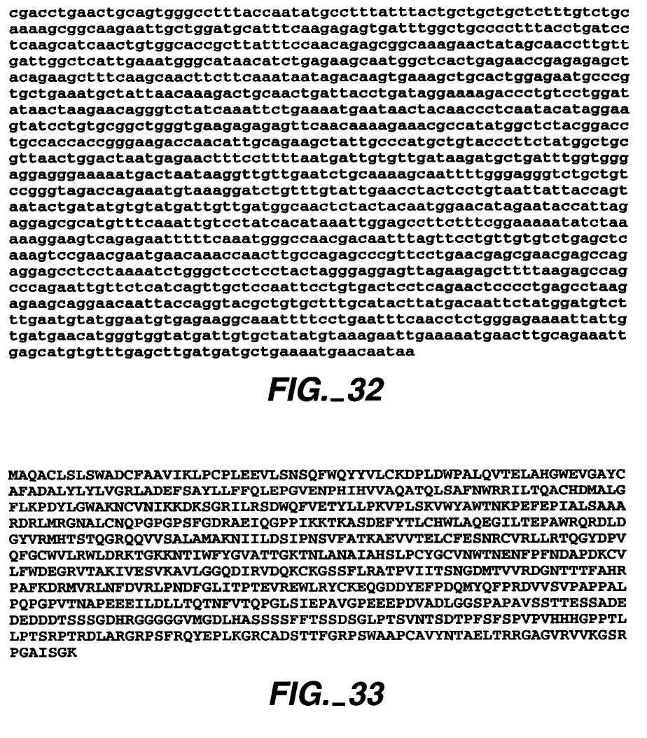

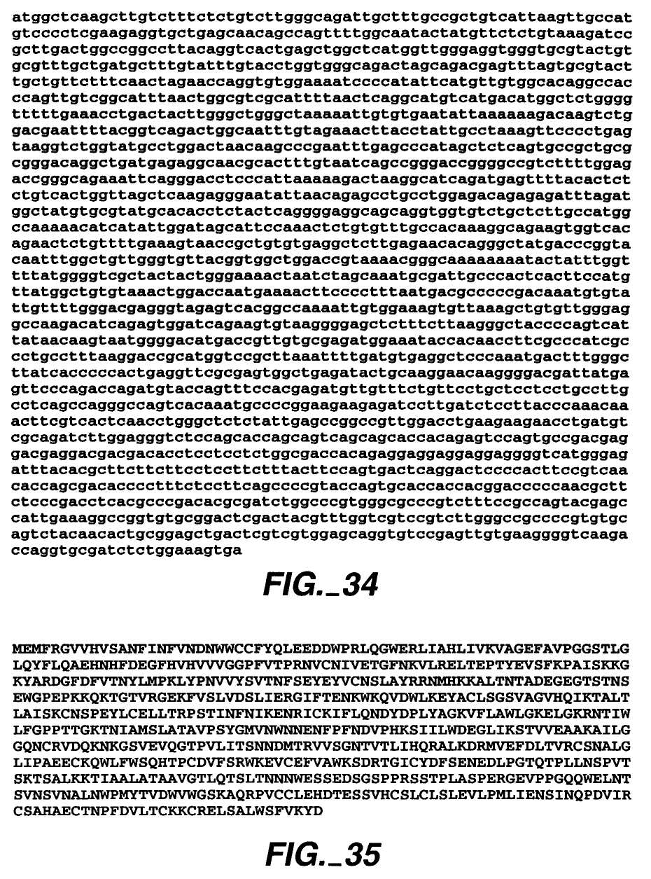

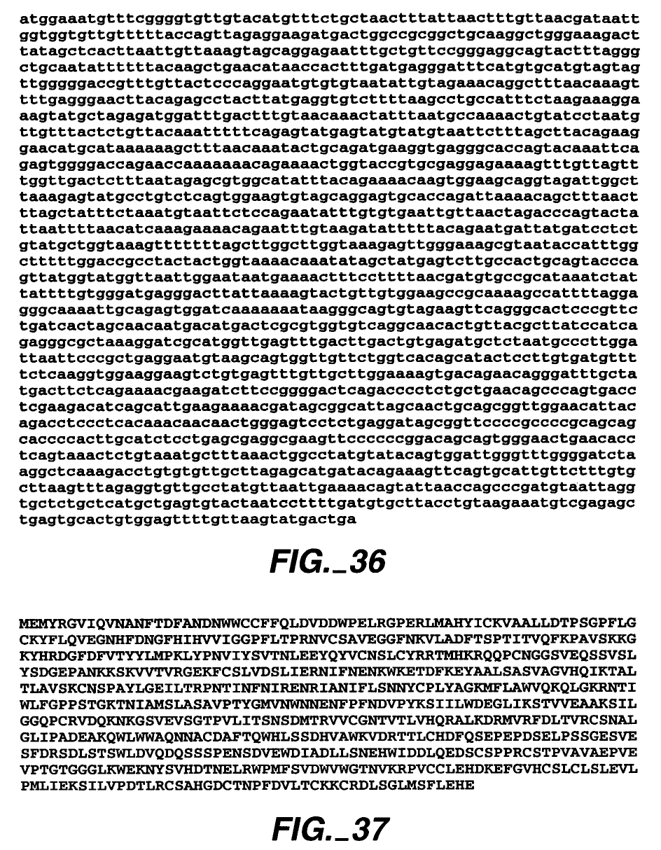

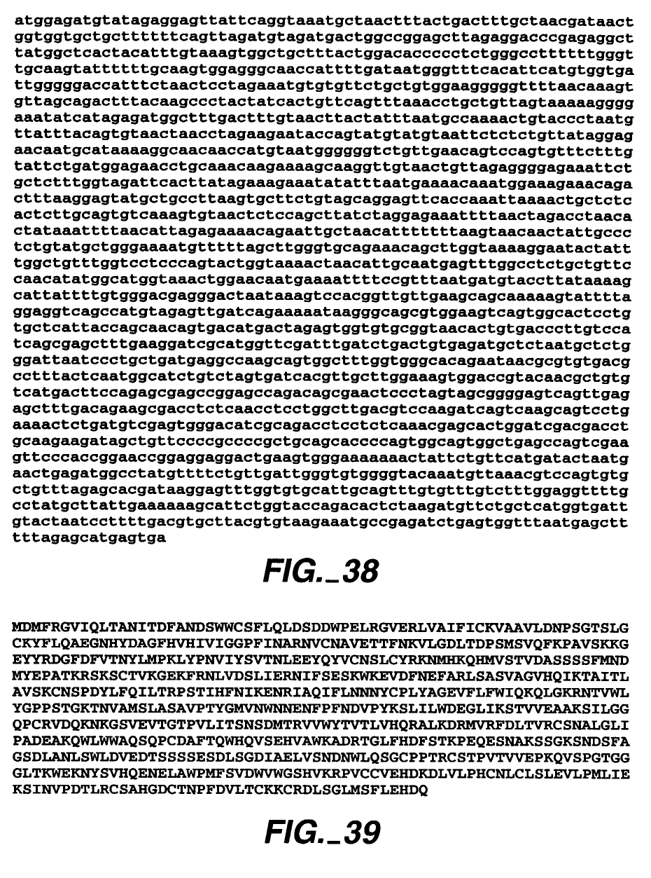

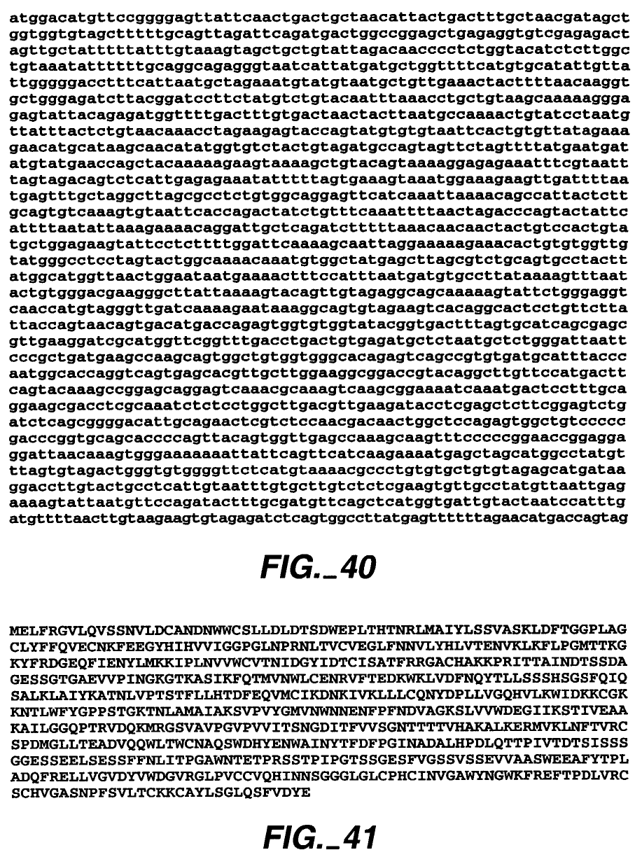

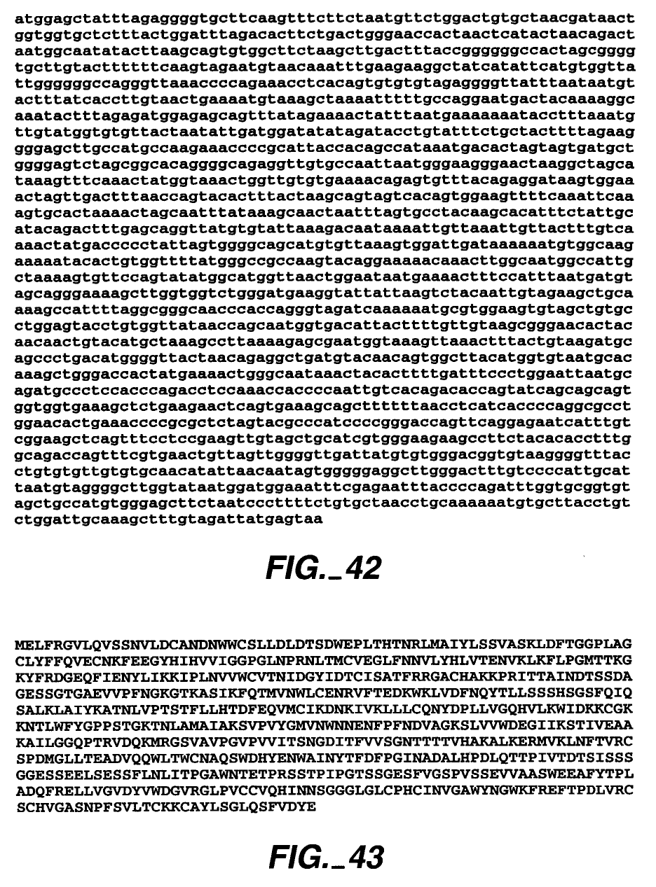

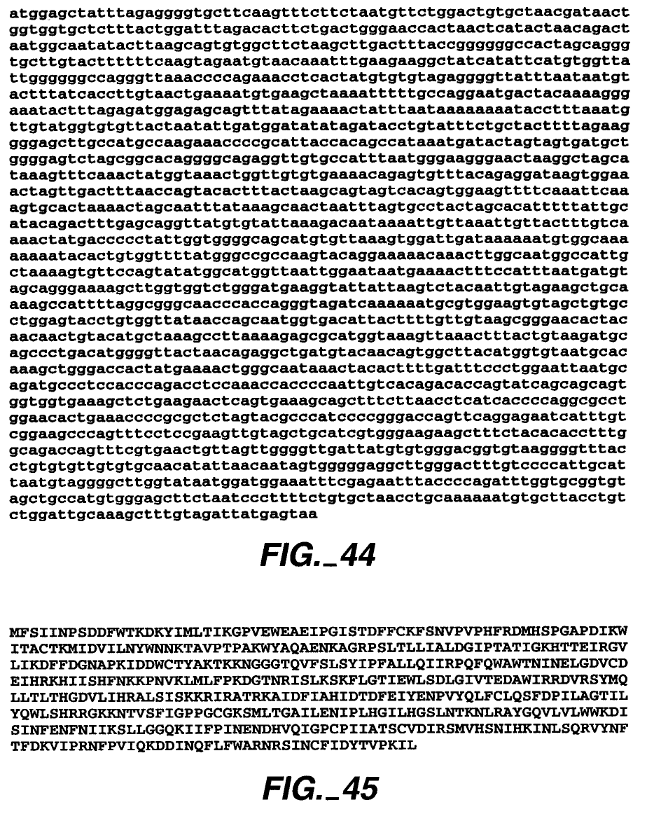

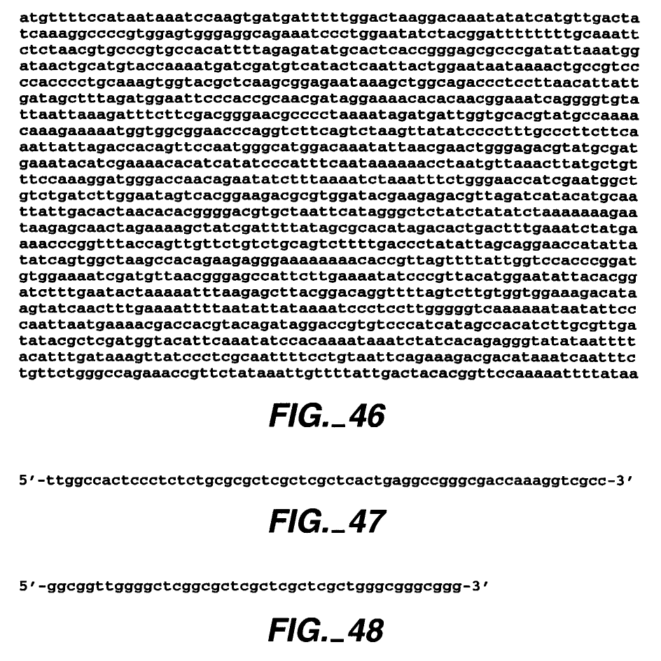

### DETAILED DESCRIPTION OF THE INVENTION

The present invention is directed to novel biochip compositions that can allow the rapid and facile creation of protein biochips that can be used in a wide variety of methods and techniques. The present invention relies on the use of nucleic acid modification enzymes that covalently and specifically bind to the sequence that encode them. Proteins of interest (for example, proteins to be arrayed for diagnostic or research purposes, as outlined below) are fused (either directly or indirectly, as outlined below) to a nucleic acid modification (NAM) enzyme. The NAM enzyme will covalently attach itself to a corresponding NAM attachment sequence (termed an enzyme attachment sequence (EAS)). Thus, by using vectors that comprising coding regions for the NAM enzyme and candidate proteins and the NAM enzyme attachment sequence, the candidate protein is covalently linked to the nucleic acid that encodes it upon translation, forming nucleic acid/protein (NAP) conjugates. These NAP conjugates thus have a nucleic acid portion and a protein portion. By using vectors that also contain capture sequences that will hybridize with capture probes on the surface of a biochip, the NAP conjugates can be “captured” or “arrayed” on the biochip. These protein biochips can then be used in a wide variety of ways, including diagnosis (e.g. detecting the presence of specific target analytes), screening (looking for target analytes that bind to specific proteins), and single-nucleotide polymorphism (SNP) analysis.

Accordingly, the present invention provides biochips comprising a substrate with an array of capture probes. By “biochip” or “array” herein is meant a substrate with a plurality of biomolecules in an array format; the size of the array will depend on the composition and end use of the array.

The biochips comprise a substrate. By “substrate” or “solid support” or other grammatical equivalents, herein is meant any material appropriate for the attachment of capture probes and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, ceramics, and a variety of other polymers. In a preferred embodiment, the substrates allow optical detection and do not themselves appreciably fluoresce.

In addition, as is known the art, the substrate may be coated with any number of materials, including polymers, such as dextrans, acrylamides, gelatins, agaraose, etc.

Preferred substrates include silicon, glass, polystyrene and other plastics and acrylics.

Generally the substrate is flat (planar), although as will be appreciated by those in the art, other configurations of substrates may be used as well, including the placement of the probes on the inside surface of a tube, for flow-through sample analysis to minimize sample volume.

The present system finds particular utility in array formats, i.e. wherein there is a matrix of addressable locations (herein generally referred to “pads”, “addresses” or “micro-locations”). By “array” herein is meant a plurality of capture probes in an array format; the size of the array will depend on the composition and end use of the array. Arrays containing from about 2 different capture probes to many thousands can be made. Generally, the array will comprise from two to as many as 100,000 or more, depending on the size of the pads, as well as the end use of the array. Preferred ranges are from about 2 to about 10,000, with from about 5 to about 1000 being preferred, and from about 10 to about 100 being particularly preferred. In some embodiments, the compositions of the invention may not be in array format; that is, for some embodiments, compositions comprising a single capture probe may be made as well. In addition, in some arrays, multiple substrates may be used, either of different or identical compositions. Thus for example, large arrays may comprise a plurality of smaller substrates.

The biochip substrates comprise an array of capture probes. By “capture probes” herein is meant nucleic acids (attached either directly or indirectly to the substrate as is more fully outlined below ) that are used to bind, e.g. hybridize, the NAP conjugates of the invention. Capture probes comprise nucleic acids. By “nucleic acid” or “oligonucleotide” or grammatical equivalents herein means at least two nucleosides covalently linked together. A nucleic acid of the present invention will generally contain phosphodiester bonds, although in some cases nucleic acid analogs are included (particularly in the case where nucleic acids are used as target analytes or test agents) that may have alternate backbones, particularly when the target molecule is a nucleic acid, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), 0-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of other elements, such as labels, or to increase the stability and half-life of such molecules in physiological environments.

As will be appreciated by those in the art, all of these nucleic acid analogs may find use in the present invention. In addition, mixtures of naturally occurring nucleic acids and analogs can be made, or, alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occuring analog structures. Thus for example the individual units of a peptide nucleic acid, each containing a base, are referred to herein as a nucleoside.

Nucleic acid arrays are known in the art, and include, but are not limited to, those made using photolithography techniques (Affymetrix GeneChip™), spotting techniques (Synteni and others), printing techniques (Hewlett Packard and Rosetta), three dimensional “gel pad” arrays (U.S. Pat. No. 5,552,270), nucleic acid arrays on electrodes and other metal surfaces (WO 98/20162; WO 10 98/12430; WO 99/57317; and WO 01/07665) microsphere arrays (U.S. Pat. No. 6,023,540; WO 00/16101; WO 99/67641; and WO 00/39587), arrays made using functionalized materials (see PhotoLink™ technology from SurModics); all of which are expressly incorporated by reference.

As will be appreciated by those in the art, the capture probes can be attached either directly to the substrate, or indirectly, through the use of polymers or through the use of microspheres.

Capture probes are designed to be substantially complementary to capture sequences of the vectors, as is described below, such that hybridization of the capture sequence and the capture probes of the present invention occurs. As outlined below, this complementarity need not be perfect; there may be any number of base pair mismatches which will interfere with hybridization between the capture sequences and the capture probes of the present invention. However, if the number of mutations is so great that no hybridization can occur under even the least stringent of hybridization conditions, the sequence is not a complementary sequence. Thus, by “substantially complementary” herein is meant that the probes are sufficiently complementary to the capture sequences to hybridize under normal reaction conditions.

As is appreciated by those in the art, the length of the probe will vary with the length of the capture sequence and the hybridization and wash conditions. Generally, oligonucleotide probes range from about 8 to about 50 nucleotides, with from about 10 to about 30 being preferred and from about 12 to about 25 being especially preferred. In some cases, very long probes may be used, e.g. 50 to 200-300 nucleotides in length.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

The capture probes of the array are used to hybridize the NAP conjugates of the invention to form arrays of candidate proteins. Thus, the invention provides libraries of nucleic acid molecules comprising nucleic acid sequences encoding fusion nucleic acids. By “fusion nucleic acid” herein is meant a plurality of nucleic acid components that are joined together. The fusion nucleic acids encode fusion polypeptides. By “fusion polypeptide” or “fusion peptide” or grammatical equivalents herein is meant a protein composed of a plurality of protein components, that while typically unjoined in their native state, are joined by their respective amino and carboxyl termini through a peptide linkage to form a single continuous polypeptide. Plurality in this context means at least two, and preferred embodiments generally utilize two components. It will be appreciated that the protein components can be joined directly or joined through a peptide linker/spacer as outlined below. In addition, it should be noted that in some embodiments, as is more fully outlined below, the fusion nucleic acids encode protein components that are not fused; for example, the fusion nucleic acid may comprise an intron that is removed, leaving two non-associated protein components, although generally the nucleic acids encoding each component are fused. Furthermore, as outlined below, additional components such as fusion partners including targeting sequences, etc. may be used.

The fusion nucleic acids encode nucleic acid modification (NAM) enzymes and candidate proteins. By “nucleic acid modification enzyme” or “NAM enzyme” herein is meant an enzyme that utilizes nucleic acids, particularly DNA, as a substrate and covalently attaches itself to nucleic acid enzyme attachment (EA) sequences. The covalent attachment can be to the base, to the ribose moiety or to the phosphate moietes. NAM enzymes include, but are not limited to, helicases, topoisomerases, polymerases, gyrases, recombinases, transposases, restriction enzymes and nucleases. As outlined below, NAM enzymes include variants. Although many DNA binding peptides are known, such as those involved in nucleic acid compaction, transcription regulators, and the like, enzymes that covalently attach to DNA, in particular peptides involved with replication, are preferred. Some NAM enzymes can form covalent linkages with DNA without nicking the DNA. For example, it is believed that enzymes involved in DNA repair recognize and covalently attach to nucleic acid regions, which can be either double-stranded or single-stranded. Such NAM enzymes are suitable for use in the fusion enzyme library. However, DNA NAM enzymes that nick DNA to form a covalent linkage, e.g., viral replication peptides, are most preferred.

Preferably, the NAM enzyme is a protein that recognizes specific sequences or conformations of a nucleic acid substrate and performs its enzymatic activity such that a covalent complex is formed with the nucleic acid substrate. Preferably, the enzyme acts upon nucleic acids, particularly DNA, in various configurations including, but not limited to, single-strand DNA, double-strand DNA, Z-form DNA, and the like.

Suitable NAM enzymes, include, but are not limited to, enzymes involved in replication such as Rep68 and Rep78 of adeno-associated viruses (MV), NS1 and H-1 of parvovirus, bacteriophage phi-29 terminal proteins, the 55 Kd adenovirus proteins, and derivatives thereof.

In a preferred embodiment, the NAM enzyme is a Rep protein. Adeno-associated viral (MV) Rep proteins are encoded by the left open reading frame of the viral genome. MV Rep proteins, such as Rep68 and Rep78, regulate AAV transcription, activate AAV replication, and have been shown to inhibit transcription of heterologous promoters (Chiorini et al., J. Virol., 68(2), 797-804 (1994), hereby incorporated by reference in its entirety). The Rep68 and Rep78 proteins act, in part, by covalently attaching to the AAV inverted terminal repeat (Prasad et al., Virology, 229, 183-192 (1997); Prasad et al., Virology 214:360 (1995); both of which are hereby incorporated by reference in their entirety). These Rep proteins act by a site-specific and strand-specific endonuclease nick at the MV origin at the terminal resolution site, followed by covalent attachment to the 5′ terminus of the nicked site via a putative tyrosine linkage. Rep68 and Rep78 result from alternate splicing of the transcript. The nucleic acid and protein sequences of Rep68 as shown in the Figures,; the nucleic acid and protein sequences of Rep78 are shown in the Figures. As is further outlined below, functional fragments and variants of Rep proteins are also included within the definition of Rep proteins; in this case, the variants preferably include nucleic acid binding activity and endonuclease activity. The corresponding enzyme attachment site for Rep68 and Rep78, discussed below, is shown in the Figures.

In a preferred embodiment, the NAM enzyme is NS1. NS1 is a non-structural protein in parvovirus, is a functional homolog of Rep78, and also covalently attaches to DNA (Cotmore et al., J. Virol., 62(3), 851-860 (1998), hereby expressly incorporated by reference). The nucleotide and amino acid sequences of NS1 are shown in the Figures. As is further outlined below, fragments and variants of NS1 proteins are also included within the definition of NS1 proteins.

In a preferred embodiment, the NAM enzyme is the parvoviral H-1 protein, which is also known to form a covalent linkage with DNA (see, for example, Tseng et al., Proc. Natl. Acad. Sci. USA, 76(11), 5539-5534 (1979), hereby expressly incorporated by reference. As is further outlined below, fragments and variants of H-1 proteins are also included within the definition of H-1 proteins.

In a preferred embodiment, the NAM enzyme is the bacteriophage phi-29 terminal protein, which is also known to form a covalent linkage with DNA (see, for example, Germendia et al., Nucleic Acid Research, 16(3), 5727-5740 (1988), hereby expressly incorporated by reference. As is further outlined below, fragments and variants of phi-29 proteins are also included within the definition of phi-29 proteins.

In a preferred embodiment, the NAM enzyme is the adenoviral 55 Kd (a55) protein, again known to form covalent linkages with DNA; see Desiderio and Kelly, J. Mol. Biol., 98, 319-337 (1981), hereby expressly incorporated by reference. As is further outlined below, fragments and variants of a55 proteins are also included within the definition of a55 proteins.

Some DNA-binding enzymes form covalent linkages upon physical or chemical stimuli such as, for example, UV-induced crosslinking between DNA and a bound protein, or camptothecin (CPT)-related chemically induced trapping of the DNA-topoisomerase I covalent complex (e.g., Hertzberg et al., J. Biol. Chem., 265, 19287-19295 (1990)). NAM enzymes that form induced covalent linkages are suitable for use in some embodiments of the present invention.

Also included with the definition of NAM enzymes of the present invention are amino acid sequence variants. These variants fall into one or more of three classes: substitutional, insertional or deletional (e.g. fragment) variants. These variants ordinarily are prepared by site specific mutagenesis of nucleotides in the DNA encoding the NAM protein, using cassette or PCR mutagenesis or other techniques well known in the art, to produce DNA encoding the variant, and thereafter expressing the recombinant DNA in cell culture as outlined herein. However, variant NAM protein fragments having up to about 100-150 residues may be prepared by in vitro synthesis or peptide ligation using established techniques. Amino acid sequence variants are characterized by the predetermined nature of the variation, a feature that sets them apart from naturally occurring allelic or interspecies variation of the NAM protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed NAM variants screened for the optimal combination of desired activity. Techniques for making substitution mutations at predetermined sites in DNA having a known sequence are well known, for example, M13 primer mutagenesis and PCR mutagenesis. Screening of the mutants is done using assays of NAM protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger, for example when unnecessary domains are removed.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the NAM protein are desired, substitutions are generally made in accordance with the following shown in chart 1:

Chart I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity as the naturally-occurring analogue, although variants also are selected to modify the characteristics of the NAM proteins as needed. Alternatively, the variant may be designed such that the biological activity of the NAM protein is altered. For example, glycosylation sites may be altered or removed. Similarly, functional mutations within the endonuclease domain or nucleic acid recognition site may be made. Furthermore, unnecessary domains may be deleted, to form fragments of NAM enzymes.

In addition, some embodiments utilize concatameric constructs to effect multivalency and increase binding kinetics or efficiency. For example, constructs containing a plurality of NAM coding regions or a plurality of EASs may be made.

Also included with the definition of NAM protein are other NAM homologs, and NAM proteins from other organisms including viruses, which are cloned and expressed as known in the art. Thus, probe or degenerate polymerase chain reaction (PCR) primer sequences may be used to find other related NAM proteins. As will be appreciated by those in the art, particularly useful probe and/or PCR primer sequences include the unique areas of the NAM nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition to nucleic acids encoding NAM enzymes, the fusion nucleic acids of the invention also encode candidate proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures, the latter being especially useful when the target molecule is a protein. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard ex vivo degradations. Chemical blocking groups or other chemical substituents may also be added. Thus, the present invention can find use in template based synthetic systems.

By “candidate protein” herein is meant a protein to be tested for binding, association or effect in an assay of the invention, including both in vitro (e.g. cell free systems) or ex vivo (within cells). Generally, as outlined below, libraries of candidate proteins are used in the fusions. As will be appreciated by those in the art, the source of the candidate protein libraries can vary, particularly depending on the end use of the system.

In a preferred embodiment, the candidate proteins are derived from cDNA libraries. The cDNA libraries can be derived from any number of different cells, particularly those outlined for host cells herein, and include cDNA libraries generated from eucaryotic and procaryotic cells, viruses, cells infected with viruses or other pathogens, genetically altered cells, etc. Preferred embodiments, as outlined below, include cDNA libraries made from different individuals, such as different patients, particularly human patients. The cDNA libraries may be complete libraries or partial libraries. Furthermore, the library of candidate proteins can be derived from a single cDNA source or multiple sources; that is, cDNA from multiple cell types or multiple individuals or multiple pathogens can be combined in a screen. The cDNA library may utilize entire cDNA constructs or fractionated constructs, including random or targeted fractionation. Suitable fractionation techniques include enzymatic, chemical or mechanical fractionation.

In a preferred embodiment, the candidate proteins are derived from genomic libraries. As above, the genomic libraries can be derived from any number of different cells, particularly those outlined for host cells herein, and include genomic libraries generated from eucaryotic and procaryotic cells, viruses, cells infected with viruses or other pathogens, genetically altered cells, etc. Preferred embodiments, as outlined below, include genomic libraries made from different individuals, such as different patients, particularly human patients. The genomic libraries may be complete libraries or partial libraries. Furthermore, the library of candidate proteins can be derived from a single genomic source or multiple sources; that is, genomic DNA from multiple cell types or multiple individuals or multiple pathogens can be combined in a screen. The genomic library may utilize entire genomic constructs or fractionated constructs, including random or targeted fractionation. Suitable fractionation techniques include enzymatic, chemical or mechanical fractionation.

In this regard, the combination of a NAM enzyme with nucleic acid derived from genomic DNA in a genetic library vector is novel. Accordingly, the present invention further provides an isolated and purified nucleic acid molecule comprising a nucleic acid sequence encoding a NAM enzyme fused to a nucleic acid sequence isolated from genomic DNA. Such an isolated and purified nucleic acid molecule is particularly useful in the present inventive methods described herein. Preferably, the isolated and purified nucleic acid molecule further comprises a splice donor sequence and splice acceptor sequence located between the nucleic acid sequence encoding the NAM enzyme and the genomic DNA. The incorporation of splice donor and splice acceptor sequences into the isolated and purified nucleic acid sequence allows formation of a transcript encoding the NAM enzyme and exons of the genomic DNA fragment. The methods of the prior art have failed to comprehend the potential of operably linking genomic DNA to a NAM enzyme such that the product of the genomic DNA can be associated with the nucleic acid molecule encoding it. One of ordinary skill in the art will appreciate that appropriate regulatory sequences can also be incorporated into the isolated and purified nucleic acid molecule.

In a preferred embodiment, the present invention also provides methods of determining open reading frames in genomic DNA. In this embodiment, the candidate protein encoded by the genomic nucleic acid is preferably fused directly to the N-terminus of the NAM enzyme, rather than at the C-terminus. Thus, if a functional NAM enzyme is produced, the genomic DNA was fused in the correct reading frame. This is particularly useful with the use of labels, as well.

In addition, the libraries may also be subsequently mutated using known techniques (exposure to mutagens, error-prone PCR, error-prone transcription, combinatorial splicing (e.g. cre-lox recombination). In this way libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

The candidate proteins may vary in size. In the case of cDNA or genomic libraries, the proteins may range from 20 or 30 amino acids to thousands, with from about 50 to 1000 being preferred and from 100 to 500 being especially preferred. When the candidate proteins are peptides, the peptides are from about 3 to about 50 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In a preferred embodiment, libraries of candidate proteins are fused to the NAM enzymes, with each member of the library comprising a different candidate protein. However, as will be appreciated by those in the art, different members of the library may be reproduced or duplicated, resulting in some libraries members being identical. The library should provide a sufficiently structurally diverse population of expression products to effect a probabilistically sufficient range of cellular responses to provide one or more cells exhibiting a desired response. Accordingly, an interaction library must be large enough so that at least one of its members will have a structure that gives it affinity for some molecule, including both protein and non-protein targets, or other factors whose activity is necessary or effective within the assay of interest. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library 5 size of 10⁷ to 10⁸ is sufficient to find structures with affinity for the target. A library of all combinations of a peptide 7 to 20 amino acids in length has the potential to code for 20⁷(10⁸) to 20²⁰ . Thus, with libraries of 1⁷ to 10⁸ the present methods allow a “working” subset of a theoretically complete interaction library for 7 amino acids, and a subset of shapes for the 20²⁰ library. Thus, in a preferred embodiment, at least 10^{6,} preferably at least 10^{7,} more preferably at least 10⁸ and most preferably at least 10⁹ different expression products are simultaneously analyzed in the subject methods. Preferred methods maximize library size and diversity.

It is important to understand that in any library system encoded by oligonucleotide synthesis one cannot have complete control over the codons that will eventually be incorporated into the peptide structure. This is especially true in the case of codons encoding stop signals (TAA, TGA, TAG). In a synthesis with NNN as the random region, there is a 3/64, or 4.69%, chance that the codon will be a stop codon. Thus, in a peptide of 10 residues, there is a high likelihood that 46.7% of the peptides will prematurely terminate. One way to alleviate this is to have random residues encoded as NNK, where K=T or G. This allows for encoding of all potential amino acids (changing their relative representation slightly), but importantly preventing the encoding of two stop residues TAA and TGA. Thus, libraries encoding a 10 amino acid peptide will have a 15.6% chance to terminate prematurely. Alternatively, fusing the candidate proteins to the C-terminus of the NAM enzyme also may be done, although in some instances, fusing to the N-terminus means that prematurely terminating proteins result in a lack of NAM enzyme which eliminates these samples from the assay.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, PDZ domains, serines, threonines, tyrosines or histidines for phosphorylation sites, etc., or to purines, etc.

In a preferred embodiment, the bias is towards peptides or nucleic acids that interact with known classes of molecules. For example, when the candidate protein is a peptide, it is known that much of intracellular signaling is carried out via short regions of polypeptides interacting with other polypeptides through small peptide domains. For instance, a short region from the HIV-1 envelope cytoplasmic domain has been previously shown to block the action of cellular calmodulin. Regions of the Fas cytoplasmic domain, which shows homology to the mastoparan toxin from Wasps, can be limited to a short peptide region with death-inducing apoptotic or G protein inducing functions. Magainin, a natural peptide derived from Xenopus, can have potent anti-tumour and anti-microbial activity. Short peptide fragments of a protein kinase C isozyme (βPKC), have been shown to block nuclear translocation of βPKC in Xenopus oocytes following stimulation. And, short SH-3 target peptides have been used as psuedosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate proteins as well.

Thus, a number of molecules or protein domains are suitable as starting points for the generation of biased randomized candidate candidate proteins. A large number of small molecule domains are known, that confer a common function, structure or affinity. In addition, as is appreciated in the art, areas of weak amino acid homology may have strong structural homology. A number of these molecules, domains, and/or corresponding consensus sequences, are known, including, but are not limited to, SH-2 domains, SH-3 domains, Pleckstrin, death domains, protease cleavage/recognition sites, enzyme inhibitors, enzyme substrates, Traf, etc. Similarly, there are a number of known nucleic acid binding proteins containing domains suitable for use in the invention. For example, leucine zipper consensus sequences are known.

In a preferred embodiment, biased SH-3 domain-binding oligonucleotides/peptides are made. SH-3 domains have been shown to recognize short target motifs (SH-3 domain-binding peptides), about ten to twelve residues in a linear sequence, that can be encoded as short peptides with high affinity for the target SH-3 domain. Consensus sequences for SH-3 domain binding proteins have been proposed. Thus, in a preferred embodiment, oligos/peptides are made with the following biases 1. XXXPPXPXX, wherein X is a randomized residue. 2. (within the positions of residue positions 11 to -2):

11 10 9 8 7 6 5 4 3 2 1

Met GTy aa11 aa10 aa9 aa8 aa7 Arg Pro Leu Pro Pro hyd

0−1 −2

Pro hyd hyd Gly Gly Pro Pro STOP (SEQ ID NO:49)

atg ggc nnk nnk nnk nnk nnk aga cct ctg cct cca sbk ggg sbk sbk gga ggc cca

cct TAA1 (SEQ ID NO:50).

In this embodiment, the N-terminus flanking region is suggested to have the greatest effects on binding affinity and is therefore entirely randomized. “Hyd” indicates a bias toward a hydrophobic residue, i.e.—Val, Ala, Gly, Leu, Pro, Arg. To encode a hydrophobically biased residue, “sbk” codon biased structure is used. Examination of the codons within the genetic code will ensure this encodes generally hydrophobic residues. s=g,c; b=t, g, c; v=a, g, c; m=a, c; k=t, g; n=a, t, g, c.

Thus, in a preferred embodiment, the candidate protein is a structural tag that will allow the isolation of target proteins with that structure. That is, in the case of leucine zippers, the fusion of the NAM enzyme to a leucine zipper sequence will allow the fusions to “zip up” with other leucine zippers, allow the quick isolation of a plurality of leucine zipper proteins. In addition, structural tags (which may only be the proteins themselves) can allow heteromultimeric protein complexes to form, that then are assayed for activity as complexes. That is, many proteins, such as many eucaryotic transcription factors, function as heteromultimeric complexes which can be assayed using the present invention.

In addition, rather than a cDNA, genomic, or random library, the candidate protein library may be a constructed library; that is, it may be built to contain only members of a defined class, or combinations of classes. For example, libraries of immunoglobulins may be built, or libraries of G-protein coupled receptors, tumor suppressor genes, proteases, transcription factors, phosphotases, kinases, etc.

The fusion nucleic acid can comprise the NAM enzyme and candidate protein in a variety of configurations, including both direct and indirect fusions, and include N- and C-terminal fusions and internal fusions.

In a preferred embodiment, the NAM enzyme and the candidate protein are directly fused. In this embodiment, a direct, in-frame fusion of the nucleic acid encoding the NAM enzyme and the candidate protein is done. Again, this may be done in several ways, including N- and C-terminal fusions and internal fusions. Thus, the NAM enzyme coding region may be 3′ or 5′ to the candidate protein coding region, or the candidate protein coding region may be inserted into a suitable position within the coding region of the NAM enzyme. In this embodiment, it may be desirable to insert the candidate protein into an external loop of the NAM enzyme, either as a direct insertion or with the replacement of several of the NAM enzyme residues. This may be particularly desirable in the case of random candidate proteins, as they frequently require some sort of scaffold or presentation structure to confer a conformationally restricted structure. For an example of this general idea using green fluorescent protein (GFP) as a scaffold for the expression of random peptide libraries, see for example WO 99/20574, expressly incorporated herein by reference. Furthermore, in this embodiment, generally only a single set of regulatory elements such as promoters are used.

In a preferred embodiment, the NAM enzyme and the candidate protein are indirectly fused. This may be done such that the components of the fusion remain attached, such as through the use of linkers, or in ways that result in the components of the fusion becoming separated. As will be appreciated by those in the art, there are a wide variety of different types of linkers that may be used, including cleavable and non-cleavable linkers; this cleavage may also occur at the level of the nucleic acid, or at the protein level.

In a preferred embodiment, linkers may be used to functionally isolate the NAM enzyme and the candidate protein. That is, a direct fusion system may sterically or functionally hinder the interaction of the candidate protein with its intended binding partner, and thus fusion configurations that allow greater degrees of freedom are useful. An analogy is seen in the single chain antibody area, where the incorporation of a linker allows functionality.

In a preferred embodiment, linkers known to confer flexibility are used. For example, useful linkers include glycine-serine polymers (including, for example, (GS)ₙ, (GSGGS)ₙ(SEQ ID NO:51) and (GGGS)ₙ(SEQ ID NO:52), where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

In a preferred embodiment, the linker is a cleavable linker. Cleavable linkers may function at the level of the nucleic acid or the protein. That is, cleavage (which in this sense means that the NAM enzyme and the candidate protein are separated) may occur during transcription, or before or after translation.

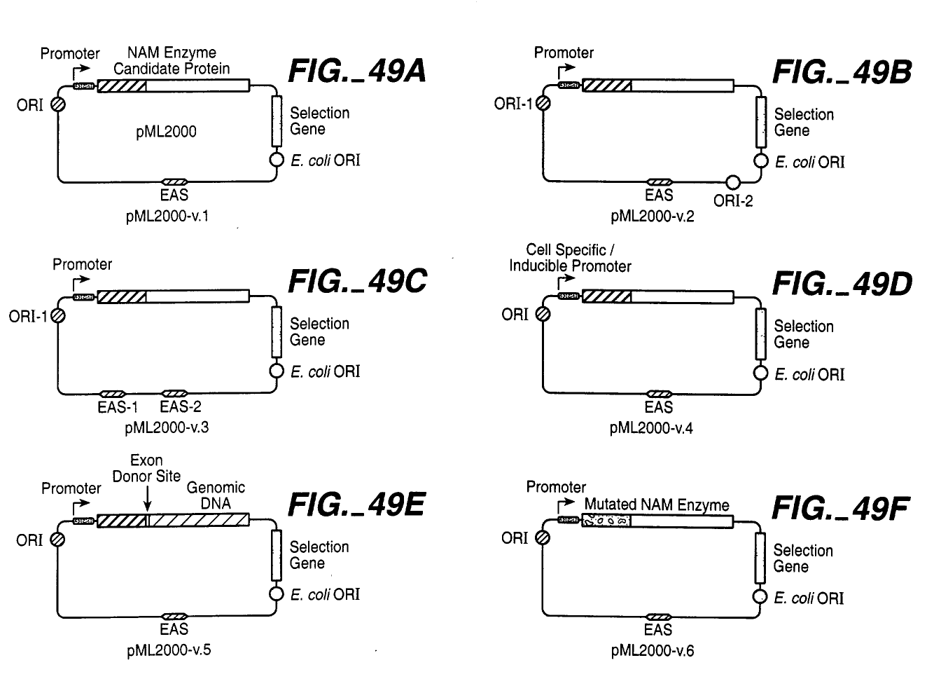

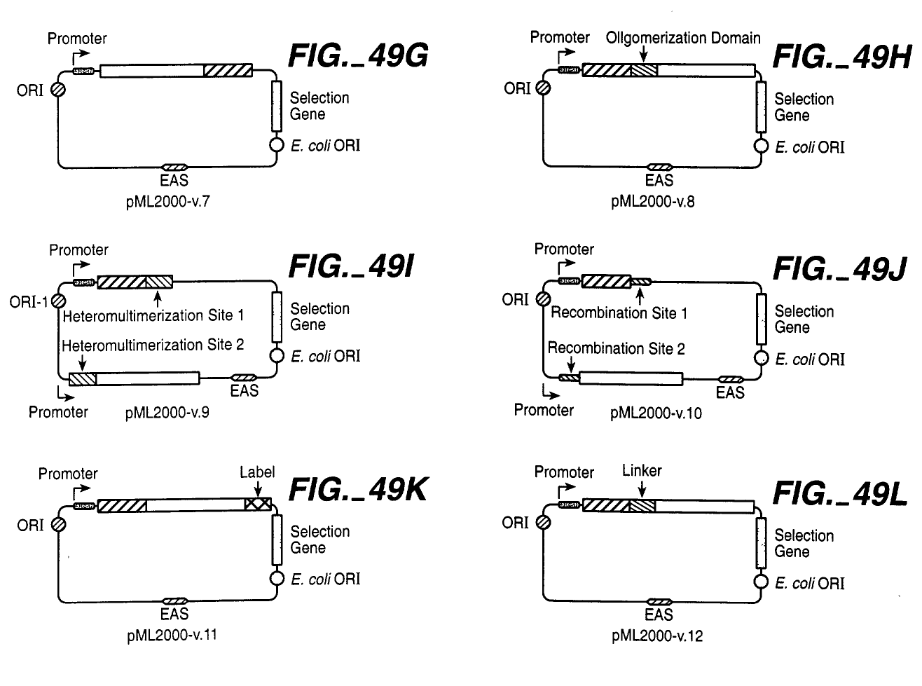

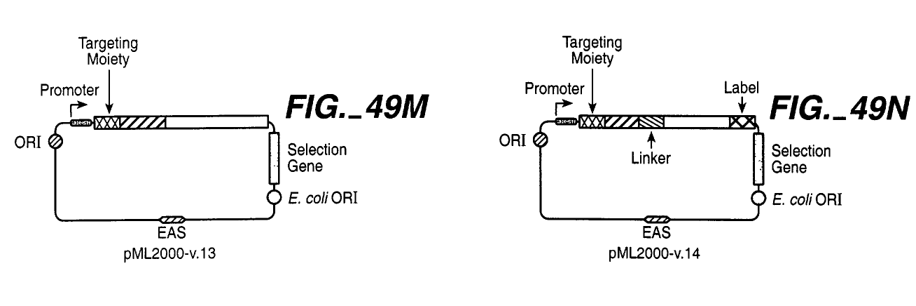

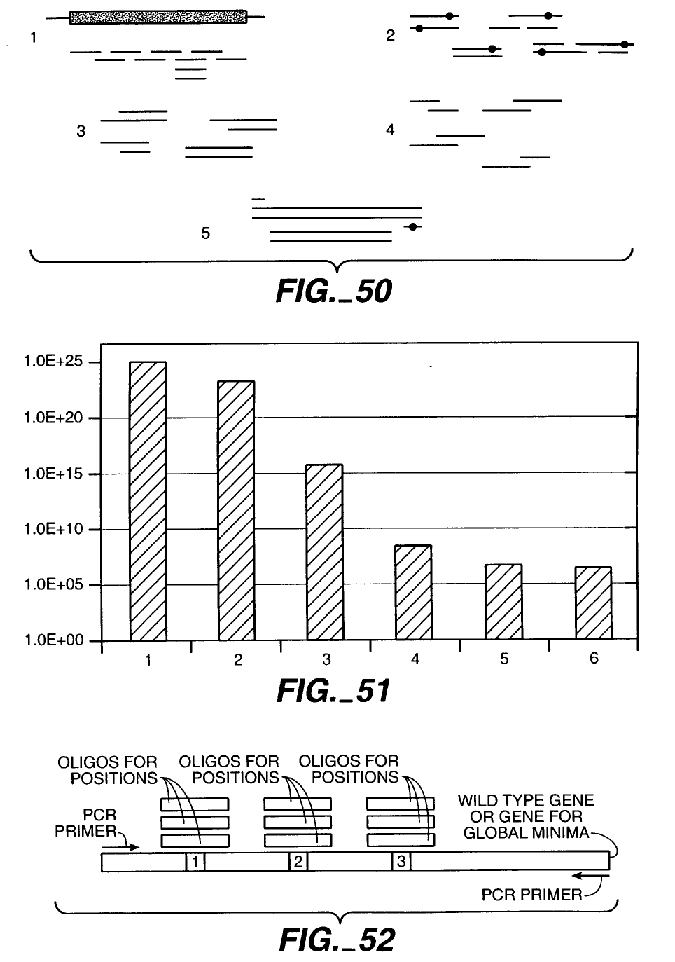

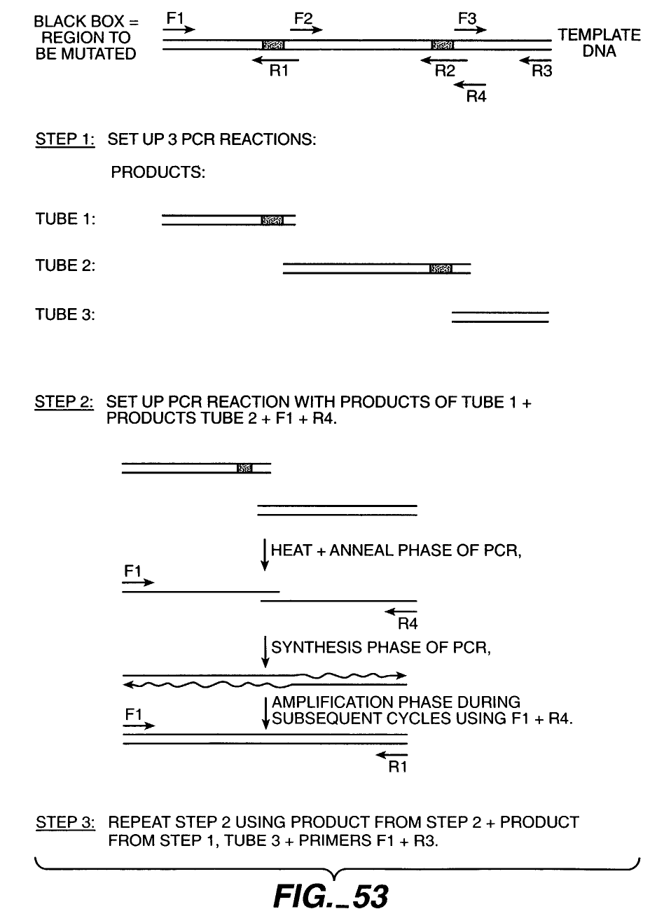

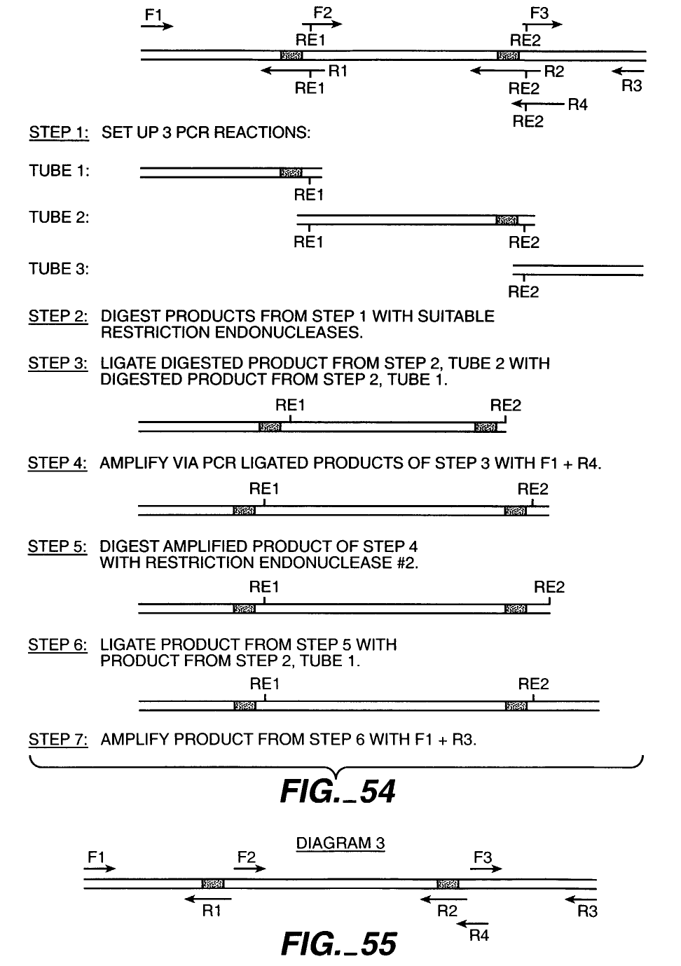

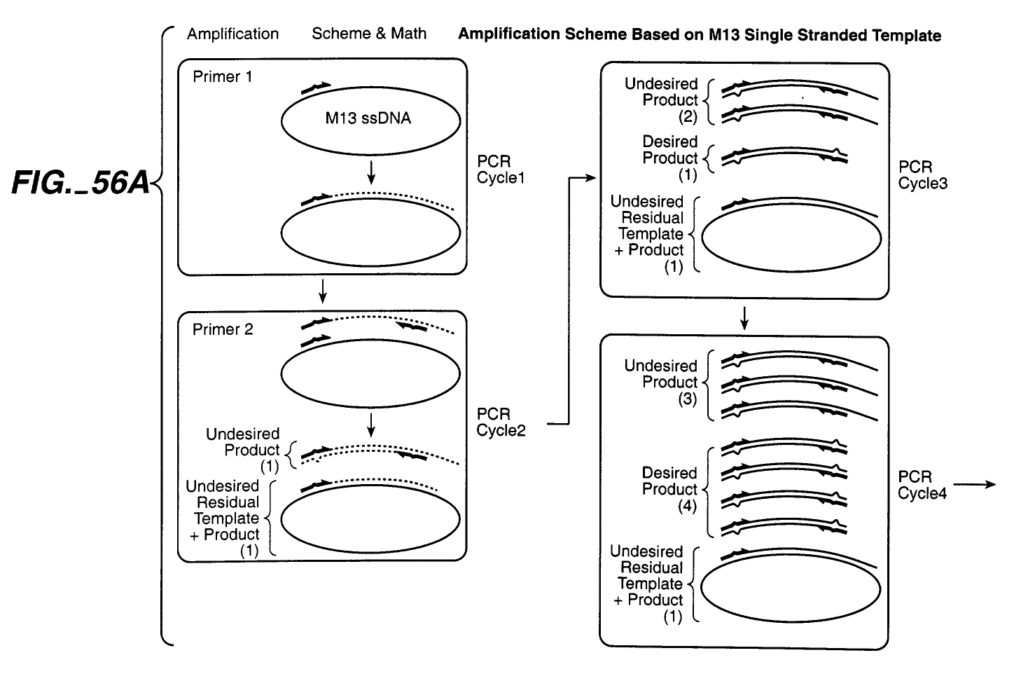

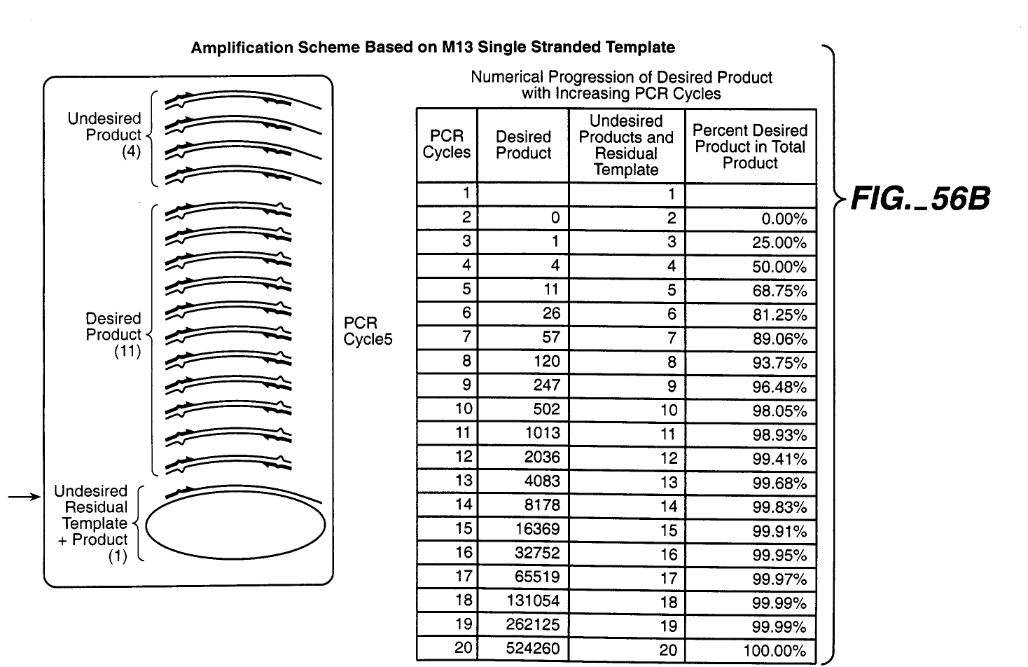

In a preferred embodiment, the cleavage occurs as a result of cleavage functionality built into the nucleic acid. In this embodiment, for example, cleavable nucleic acid sequences, or sequences that will disrupt the nucleic acid, can be used. For example, intron sequences that the cell will remove can be placed between the coding region of the NAM enzyme and the candidate protein. See FIG. 49, which depicts two different vectors comprising exon donor sites and splice recipient sites.

In a preferred embodiment, the linkers are heterodimerization domains, as depicted in FIG. 49. In this embodiment, both the NAM enzyme and the candidate protein are fused to heterodimerization domains (or multimeric domains, if multivalency is desired), to allow association of these two proteins after translation.

In a preferred embodiment, cleavable protein linkers are used. In this embodiment, the fusion nucleic acids include coding sequences for a protein sequence that may be subsequently cleaved, generally by a protease. As will be appreciated by those in the art, cleavage sites directed to ubiquitous proteases, e.g. those that are constitutively present in most or all of the host cells of the system, can be used. Alternatively, cleavage sites that correspond to cell-specific proteases may be used. Similarly, cleavage sites for proteases that are induced only during certain cell cycles or phases or are signal specific events may be used as well.

There are a wide variety of possible proteinaceous cleavage sites known. For example, sequences that are recognized and cleaved by a protease or cleaved after exposure to certain chemicals are considered cleavable linkers. This may find particular use in in vitro systems, outlined below, as exogeneous enzymes can be added to the milieu or the NAP conjugates may be purified and the cleavage agents added. For example, cleavable linkers include, but are not limited to, the prosequence of bovine chymosin, the prosequence of subtilisin, the 2a site (Ryan et al., J. Gen. Virol. 72:2727 (1991); Ryan et al., EMBO J. 13:928 (1994); Donnelly et al., J. Gen. Virol. 78:13 (1997); Hellen et al., Biochem, 28(26):9881 (1989); and Mattion et al., J. Virol. 70:8124 (1996)), prosequences of retroviral proteases including human immunodeficiency virus protease and sequences recognized and cleaved by trypsin (EP 578472, Takasuga et al., J. Biochem. 112(5)652 (1992)) factor Xa (Gardella et al., J. Biol. Chem. 265(26):15854 (1990), WO 9006370), collagenase (J03280893, Tajima et al., J. Ferment. Bioeng. 72(5):362 (1991), WO 9006370), clostripain (EP 578472), subtilisin (including mutant H64A subtilisin, Forsberg et al., J. Protein Chem. 10(5):517 (1991), chymosin, yeast KEX2 protease (Bourbonnais et al., J. Bio. Chem. 263(30):15342 (1988), thrombin (Forsberg et al., supra; Abath et al., BioTechniques 10(2):178 (1991)), Staphylococcus aureus V8 protease or similar endoproteinase-Glu-C to cleave after Glu residues (EP 578472, Ishizaki et al., Appl. Microbiol. Biotechnol. 36(4):483 (1992)), cleavage by NIa proteainase of tobacco etch virus (Parks et al., Anal. Biochem. 216(2):413 (1994)), endoproteinase-Lys-C (U.S. Pat. No. 4,414,332) and endoproteinase-Asp-N, Neisseria type 2 IgA protease (Pohiner et al., Bio/Technology 10(7):799-804 (1992)), soluble yeast endoproteinase yscF (EP 467839), chymotrypsin (Altman et al., Protein Eng. 4(5):593 (1991)), enteropeptidase (WO 9006370), lysostaphin, a polyglycine specific endoproteinase (EP 316748), and the like. See e.g. Marston, F. A. O. (1986) Biol. Chem. J. 240, 1-12. Particular amino acid sites that serve as chemical cleavage sites include, but are not limited to, methionine for cleavage by cyanogen bromide (Shen, PNAS USA 81:4627 (1984); Kempe et al., Gene 39:239 (1985); Kuliopulos et al., J. Am. Chem. Soc. 116:4599 (1994); Moks et al., Bio/Technology 5:379 (1987); Ray et al., Bio/Technology 11:64 (1993)), acid cleavage of an Asp-Pro bond (Wingender et al., J. Biol. Chem. 264(8):4367 (1989); Gram et al., Bio/Technology 12:1017 (1994)), and hydroxylamine cleavage at an Asn-Gly bond (Moks supra). In addition to the NAM enzymes, candidate proteins, and linkers, the fusion nucleic acids may comprise additional coding sequences for other functionalities. As will be appreciated by those in the art, the discussion herein is directed to fusions of these other components to the fusion nucleic acids described herein; however, they may also be unconnected to the fusion protein and rather be a component of the expression vector comprising the fusion nucleic acid, as is generally outlined below.

Thus, in a preferred embodiment, the fusions are linked to a fusion partner. By “fusion partner” or “functional group” herein is meant a sequence that is associated with the candidate candidate protein, that confers upon all members of the library in that class a common function or ability. Fusion partners can be heterologous (i.e. not native to the host cell), or synthetic (not native to any cell). Suitable fusion partners include, but are not limited to: a) presentation structures, as defined below, which provide the candidate proteins in a conformationally restricted or stable form, including hetero- or homodimerization or multimerization sequences; b) targeting sequences, defined below, which allow the localization of the candidate proteins into a subcellular or extracellular compartment or be incorporated into infected organisms, such as those infected by viruses or pathogens; c) rescue sequences as defined below, which allow the purification or isolation of the NAP conjugates; d) stability sequences, which confer stability or protection from degradation to the candidate protein or the nucleic acid encoding it, for example resistance to proteolytic degradation; e) linker sequences; f) any number of heterologous proteins, particularly for labeling purposes as described herein; or g) any combination of a), b), c), d), e) and f), as well as linker sequences as needed.

In a preferred embodiment, the fusion partner is a presentation structure. By “presentation structure” or grammatical equivalents herein is meant a sequence, which, when fused to candidate proteins, causes the candidate proteins to assume a conformationally restricted form. This is particularly useful when the candidate proteins are random, biased random or pseudorandom peptides. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems.

Thus, synthetic presentation structures, i.e. artificial polypeptides, are capable of presenting a randomized peptide as a conformationally-restricted domain. Generally such presentation structures comprise a first portion joined to the N-terminal end of the randomized peptide, and a second portion joined to the C-terminal end of the peptide; that is, the peptide is inserted into the presentation structure, although variations may be made, as outlined below. To increase the functional isolation of the randomized expression product, the presentation structures are selected or designed to have minimal biologically activity when expressed in the target cell.

Preferred presentation structures maximize accessibility to the peptide by presenting it on an exterior loop. Accordingly, suitable presentation structures include, but are not limited to, minibody structures, dimerization sequences, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

In a preferred embodiment, the presentation structure is a coiled-coil structure, allowing the presentation of the randomized peptide on an exterior loop. See, for example, Myszka et al., Biochem. 33:2362-2373 (1994), hereby incorporated by reference). Using this system investigators have isolated peptides capable of high affinity interaction with the appropriate target. In general, coiled-coil structures allow for between 6 to 20 randomized positions.

A preferred coiled-coil presentation structure is as follows: MGCAALESEVSALESEVASLESEVAALGRGDMPLAAVKSKLSAVKSKLASVKSKLAACGPP (SEQ ID

NO:53). The underlined regions represent a coiled-coil leucine zipper region defined previously (see Martin et al., EMBO J. 13(22):5303-5309 (1994), incorporated by reference). The bolded GRGDMP (SEQ ID NO:54) region represents the loop structure and when appropriately replaced with randomized peptides (i.e., candidate proteins, generally depicted herein as (X)ₙ, where X is an amino acid residue and n is an integer of at least 5 or 6) can be of variable length. The replacement of the bolded region is facilitated by encoding restriction endonuclease sites in the underlined regions, which allows the direct incorporation of randomized oligonucleotides at these positions. For example, a preferred embodiment generates a Xhol site at the double underlined LE site and a HindIII site at the double-underlined KL site.

In a preferred embodiment, the presentation structure is a minibody structure. A “minibody” is essentially composed of a minimal antibody complementarity region. The minibody presentation structure generally provides two randomizing regions that in the folded protein are presented along a single face of the tertiary structure. See for example Bianchi et al., J. Mol. Biol. 236(2):649-59 (1994) and references cited therein, all of which are incorporated by reference). Investigators have shown this minimal domain is stable in solution and have used phage selection systems in combinatorial libraries to select minibodies with peptide regions exhibiting high affinity, Kd=10⁻⁷, for the pro-inflammatory cytokine IL-6.

A preferred minibody presentation structure is as follows: MGRNSQATSGFTFSHFYMEWVRGGEYIMSRHKHNKYTTEYSASVKGRYIVSRDTSQSILYLQKKKG PP (SEQ ID NO:55). The bold, underline regions are the regions which may be randomized. The italicized phenylalanine must be invariant in the first randomizing region. The entire peptide is cloned in a three-oligonucleotide variation of the coiled-coil embodiment, thus allowing two different randomizing regions to be incorporated simultaneously. This embodiment utilizes non-palindromic BstXI sites on the termini.

In a preferred embodiment, the presentation structure is a sequence that contains generally two cysteine residues, such that a disulfide bond may be formed, resulting in a conformationally constrained sequence. This embodiment is particularly preferred when secretory targeting sequences are used. As will be appreciated by those in the art, any number of random sequences, with or without spacer or linking sequences, may be flanked with cysteine residues. In other embodiments, effective presentation structures may be generated by the random regions themselves. For example, the random regions may be “doped” with cysteine residues which, under the appropriate redox conditions, may result in highly crosslinked structured conformations, similar to a presentation structure. Similarly, the randomization regions may be controlled to contain a certain number of residues to confer β-sheet or α-helical structures.

In one embodiment, the presentation structure is a dimerization or multimerization sequence. A dimerization sequence allows the non-covalent association of one candidate protein to another candidate protein, including peptides, with sufficient affinity to remain associated under normal physiological conditions. This effectively allows small libraries of candidate protein (for example, 10⁴) to become large libraries if two proteins per cell are generated which then dimerize, to form an effective library of 10⁸(10⁴×10⁴). It also allows the formation of longer proteins, if needed, or more structurally complex molecules. The dimers may be homo- or heterodimers.

Dimerization sequences may be a single sequence that self-aggregates, or two sequences. That is, nucleic acids encoding both a first candidate protein with dimerization sequence 1, and a second candidate protein with dimerization sequence 2, such that upon introduction into a cell and expression of the nucleic acid, dimerization sequence 1 associates with dimerization sequence 2 to form a new structure.

Suitable dimerization sequences will encompass a wide variety of sequences. Any number of protein-protein interaction sites are known. In addition, dimerization sequences may also be elucidated using standard methods such as the yeast two, hybrid system, traditional biochemical affinity binding studies, or even using the present methods.

In a preferred embodiment, the fusion partner is a targeting sequence. As will be appreciated by those in the art, the localization of proteins within a cell is a simple method for increasing effective concentration and determining function. For example, RAF1 when localized to the mitochondrial membrane can inhibit the anti-apoptotic effect of BCL-2. Similarly, membrane bound Sos induces Ras mediated signaling in T-lymphocytes. These mechanisms are thought to rely on the principle of limiting the search space for ligands, that is to say, the localization of a protein to the plasma membrane limits the search for its ligand to that limited dimensional space near the membrane as opposed to the three dimensional space of the cytoplasm. Alternatively, the concentration of a protein can also be simply increased by nature of the localization. Shuttling the proteins into the nucleus confines them to a smaller space thereby increasing concentration. Finally, the ligand or target may simply be localized to a specific compartment, and inhibitors must be localized appropriately.

Thus, suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signalling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane or within pathogens or viruses that have infected the cell; and b) extracellular locations via a secretory signal. Particularly preferred is localization to either subcellular locations or to the outside of the cell via secretion.

In a preferred embodiment, the targeting sequence is a nuclear localization signal (NLS). NLSes are generally short, positively charged (basic) domains that serve to direct the entire protein in which they occur to the cell's nucleus. Numerous NLS amino acid sequences have been reported including single basic NLSes such as that of the SV40 (monkey virus) large T Antigen (Pro Lys Lys Lys Arg Lys Val (SEQ ID NO:56)), Kalderon (1984), et al., Cell, 39:499-509; the human retinoic acid receptor-β nuclear localization signal (ARRRRP (SEQ ID NO:57)); NFκB p50 (EEVQRKRQKL (SEQ ID NO:58); Ghosh et al., Cell 62:1019 (1990); NFκB p65 (EEKRKRTYE (SEQ ID NO:59); Nolan et al., Cell 64:961 (1991); and others (see for example Boulikas, J. Cell. Biochem. 55(1):32-58 (1994), hereby incorporated by reference) and double basic NLSes exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp (SEQ ID NO:60)), Dingwall, et al., Cell, 30:449-458, 1982 and Dingwall, et al., J. Cell Biol., 107:641-849; 1988). Numerous localization studies have demonstrated that NLSes incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. NatI. Acad. Sci. USA, 84:6795-6799, 1987; Galileo, et al., Proc. Natl. Acad. Sci. USA, 87:458-462, 1990.

In a preferred embodiment, the targeting sequence is a membrane anchoring signal sequence. This is particularly useful since many parasites and pathogens bind to the membrane, in addition to the fact that many intracellular events originate at the plasma membrane. Thus, membrane-bound peptide libraries are useful for both the identification of important elements in these processes as well as for the discovery of effective inhibitors. In addition, many drugs interact with membrane associated proteins. The invention provides methods for presenting the candidate proteins extracellularly or in the cytoplasmic space. For extracellular presentation, a membrane anchoring region is provided at the carboxyl terminus of the candidate protein. The candidate protein region is expressed on the cell surface and presented to the extracellular space, such that it can bind to other surface molecules (affecting their function) or molecules present in the extracellular medium. The binding of such molecules could confer function on the cells expressing a peptide that binds the molecule. The cytoplasmic region could be neutral or could contain a domain that, when the extracellular candidate protein region is bound, confers a function on the cells (activation of a kinase, phosphatase, binding of other cellular components to effect function). Similarly, the candidate protein-containing region could be contained within a cytoplasmic region, and the transmembrane region and extracellular region remain constant or have a defined function.

In addition, it should be noted that in this embodiment, as well as others outlined herein, it is possible that the formation of the NAP conjugate happens after the screening; that is, having the fusion protein expressed on the extracellular surface means that it may not be available for binding to the nucleic acid. However, this may be done later, with lysis of the cell.

Membrane-anchoring sequences are well known in the art and are based on the genetic geometry of mammalian transmembrane molecules. Peptides are inserted into the membrane based on a signal sequence (designated herein as ssTM) and require a hydrophobic transmembrane domain (herein TM). The transmembrane proteins are inserted into the membrane such that the regions encoded 5′ of the transmembrane domain are extracellular and the sequences 3′ become intracellular. Of course, if these transmembrane domains are placed 5′ of the variable region, they will serve to anchor it as an intracellular domain, which may be desirable in some embodiments. ssTMs and TMs are known for a wide variety of membrane bound proteins, and these sequences may be used accordingly, either as pairs from a particular protein or with each component being taken from a different protein, or alternatively, the sequences may be synthetic, and derived entirely from consensus as artificial delivery domains.

As will be appreciated by those in the art, membrane-anchoring sequences, including both ssTM and TM, are known for a wide variety of proteins and any of these may be used. Particularly preferred membrane-anchoring sequences include, but are not limited to, those derived from CD8, ICAM-2, IL-8 R, CD4 and LFA-1.

Useful sequences include sequences from: 1) class I integral membrane proteins such as IL-2 receptor beta-chain (residues 1-26 are the signal sequence, 241-265 are the transmembrane residues; see Hatakeyama et al., Science 244:551 (1989) and von Heijne et al, Eur. J. Biochem. 174:671 (1988)) and insulin receptor beta chain (residues 1-27 are the signal, 957-959 are the transmembrane domain and 960-1382 are the cytoplasmic domain; see Hatakeyama, supra, and Ebina et al., Cell 40:747 (1985)); 2) class II integral membrane proteins such as neutral endopeptidase (residues 29-51 are the transmembrane domain, 2-28 are the cytoplasmic domain; see Malfroy et al., Biochem. Biophys. Res. Commun. 144:59 (1987)); 3) type III proteins such as human cytochrome P450 NF25 (Hatakeyama, supra); and 4) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (MASPLTRFLSLNLLLLGESILGSGEAKPQAP (SEQ ID NO:61); Nakauchi et al., PNAS USA 82:5126 (1985) and 1-21 in the case of ICAM-2 (MSSFGYRTLTVALFTLICCPG (SEQ ID NO:62); Staunton et al., Nature (London) 339:61 (1989)). These leader sequences deliver the construct to the membrane while the hydrophobic transmembrane domains, placed 3′ of the random candidate region, serve to anchor the construct in the membrane. These transmembrane domains are encompassed by amino acids 145-195 from CD8 (PQRPEDCRPRGSVKGTGLDFACDIYIWAPLAGICVALLLSLIITLICYHSR (SEQ ID NO:63); Nakauchi, supra) and 224-256 from ICAM-2 (MVIIVTVVSVLLSLFVTSVLLCFIFGQHLRQQR (SEQ ID NO:64); Staunton, supra).

Alternatively, membrane anchoring sequences include the GPI anchor, which results in a covalent bond between the molecule and the lipid bilayer via a glycosyl-phosphatidylinositol bond for example in DAF (PNKGSGTTSGTTRLLSGHTCFTLTGLLGTLVTMGLLT (SEQ ID NO:65), with the bolded serine the site of the anchor; see Homans et al., Nature 333(6170):269-72 (1988), and Moran et al., J. Biol. Chem. 266:1250 (1991)). In order to do this, the GPI sequence from Thy-1 can be cassetted 3′ of the variable region in place of a transmembrane sequence.

Similarly, myristylation sequences can serve as membrane anchoring sequences. It is known that the myristylation of c-src recruits it to the plasma membrane. This is a simple and effective method of membrane localization, given that the first 14 amino acids of the protein are solely responsible for this function: MGSSKSKPKDPSQR (SEQ ID NO:66) (see Cross et al., Mol. Cell. Biol. 4(9):1834 (1984); Spencer et al., Science 262:1019-1024 (1993), both of which are hereby incorporated by reference). This motif has already been shown to be effective in the localization of reporter genes and can be used to anchor the zeta chain of the TCR. This motif is placed 5′ of the variable region in order to localize the construct to the plasma membrane. Other modifications such as palmitoylation can be used to anchor constructs in the plasma membrane; for example, palmitoylation sequences from the G protein-coupled receptor kinase GRK6 sequence (LLQRLFSRQDCCGNCSDSEEELPTRL (SEQ ID NO:67), with the bold cysteines being palmitolyated; Stoffel et al., J. Biol. Chem 269:27791 (1994)); from rhodopsin (KQFRNCMLTSLCCGKNPLGD (SEQ ID NO:68); Barnstable et al., J. Mol. Neurosci. 5(3):207 (1994)); and the p21 H-ras 1 protein (LNPPDESGPGCMSCKCVLS (SEQ ID NO:69); Capon et al., Nature 302:33 (1983)).

In a preferred embodiment, the targeting sequence is a lysozomal targeting sequence, including, for example, a lysosomal degradation sequence such as Lamp-2 (KFERQ (SEQ ID NO:70); Dice, Ann. N. Y. Acad. Sci. 674:58 (1992); or lysosomal membrane sequences from Lamp-1 (MLIPIAGFFALAGLVLIVLIAYLIGRKRSHAGYQTI (SEQ ID NO:71), Uthayakumar et al., Cell. Mol. Biol. Res. 41:405 (1995)) or Lamp-2 (LVPIAVGAALAGVLILVLLAYFIGLKHHHAGYEQF (SEQ ID NO:72), Konecki et la., Biochem. Biophys. Res. Comm. 205:1-5 (1994), both of which show the transmembrane domains in italics and the cytoplasmic targeting signal underlined).

Alternatively, the targeting sequence may be a mitrochondrial localization sequence, including mitochondrial matrix sequences (e.g. yeast alcohol dehydrogenase III; MLRTSSLFTRRVQPSLFSRNILRLQST (SEQ ID NO:73); Schatz, Eur. J. Biochem. 165:1-6 (1987)); mitochondrial inner membrane sequences (yeast cytochrome c oxidase subunit IV; MLSLRQSIRFFKPATRTLCSSRYLL (SEQ ID NO:74); Schatz, supra); mitochondrial intermembrane space sequences (yeast cytochrome c1; MFSMLSKRWAQRTLSKSFYSTATGAASKSGKLTQKLVTAGVAAAGITASTLLYADSLTAEAMTA (SEQ ID NO:75); Schatz, supra) or mitochondrial outer membrane sequences (yeast 70 kD outer membrane protein; MKSFITRNKTAILATVAATGTAIGAYYYYNQLQQQQQRGKK (SEQ ID NO:76); Schatz, supra).

The target sequences may also be endoplasmic reticulum sequences, including the sequences from calreticulin (KDEL (SEQ ID NO:77); Pelham, Royal Society London Transactions B; 1-10 (1992)) or adenovirus E3/19K protein (LYLSRRSFIDEKKMP (SEQ ID NO:78); Jackson et al., EMBO J. 9:3153 (1990).

Furthermore, targeting sequences also include peroxisome sequences (for example, the peroxisome matrix sequence from Luciferase; SKL; Keller et al., PNAS USA 4:3264 (1987)); farnesylation sequences (for example, P21 H-ras 1; LNPPDESGPGCMSCKCVLS (SEQ ID NO:79), with the bold cysteine farnesylated; Capon, supra); geranylgeranylation sequences (for example, protein rab-5A; LTEPTQPTRNQCCSN (SEQ ID NO:80), with the bold cysteines geranylgeranylated; Farnsworth, PNAS USA 91:11963 (1994)); or destruction sequences (cyclin B1; RTALGDIGN (SEQ ID NO:81); Klotzbucher et al., EMBO J. 1:3053 (1996)).

In a preferred embodiment, the targeting sequence is a secretory signal sequence capable of effecting the secretion of the candidate protein. There are a large number of known secretory signal sequences which are placed 5′ to the variable peptide region, and are cleaved from the peptide region to effect secretion into the extracellular space. Secretory signal sequences and their transferability to unrelated proteins are well known, e.g., Silhavy, et al. (1985) Microbiol. Rev. 49, 398-418. This is particularly useful to generate a peptide capable of binding to the surface of, or affecting the physiology of, a target cell that is other than the host cell. In this manner, target cells grown in the vicinity of cells caused to express the library of peptides, are bathed in secreted peptide. Target cells exhibiting a physiological change in response to the presence of a peptide, e.g., by the peptide binding to a surface receptor or by being internalized and binding to intracellular targets, and the secreting cells are localized by any of a variety of selection schemes and the peptide causing the effect determined. Exemplary effects include variously that of a designer cytokine (i.e., a stem cell factor capable of causing hematopoietic stem cells to divide and maintain their totipotential), a factor causing cancer cells to undergo spontaneous apoptosis, a factor that binds to the cell surface of target cells and labels them specifically, etc.

Similar to the membrane-anchored embodiment, it is possible that the formation of the NAP conjugate happens after the screening; that is, having the fusion protein secreted means that it may not be available for binding to the nucleic acid. However, this may be done later, with lysis of the cell.

Suitable secretory sequences are known, including signals from IL-2 (MYRMQLLSCIALSLALVTNS (SEQ ID NG:82); Villinger et al., J. Immunol. 155:3946 (1995)), growth hormone (MATGSRTSLLLAFGLLCLPWLQEGSAFPT (SEQ ID NO:83); Roskam et al., Nucleic Acids Res. 7:305 (1979)); preproinsulin (MALWMRLLPLLALLALWGPDPAAAFVN (SEQ ID NO:84); Bell et al., Nature 284:26 (1980)); and influenza HA protein (MKAKLLVLLYAFVAGDQI (SEQ ID NO:85); Sekikawa et al., PNAS 80:3563)), with cleavage between the non-underlined-underlined junction. A particularly preferred secretory signal sequence is the signal leader sequence from the secreted cytokine IL-4, which comprises the first 24 amino acids of IL-4 as follows: MGLTSQLLPPLFFLLACAGNFVHG (SEQ ID NO:86).

In a preferred embodiment, the fusion partner is a rescue sequence (sometimes also referred to herein as “purification tags” or “retrieval properties”). A rescue sequence is a sequence which may be used to purify or isolate either the candidate protein or the NAP conjugate. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST. Rescue sequences can be utilized on the basis of a binding event, an enzymatic event, a physical property or a chemical property.

Alternatively, the rescue sequence may be a unique oligonucleotide sequence which serves as a probe target site to allow the quick and easy isolation of the construct, via PCR, related techniques, or hybridization.

In a preferred embodiment, the fusion partner is a stability sequence to confer stability to the candidate protein or the nucleic acid encoding it. Thus, for example, peptides may be stabilized by the incorporation of glycines after the initiation methionine (MG or MGG0), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate protein structure. Thus, preferred stability sequences are as follows: MG(X)ₙGGPP (SEQ ID NO:87), where X is any amino acid and n is an integer of at least four.

In addition, linker sequences, as defined above, may be used in any configuration as needed.

In a preferred embodiment, the fusion partner is a heterologous protein. Any number of different proteins may be added for a variety of reasons, including for labeling purposes as outlined below. Particularly suitable heterologous proteins for fusing with the candidate proteins include autofluorescent proteins. Preferred fluorescent molecules include but are not limited to green fluorescent protein (GFP; from Aquorea and Renilla species), blue fluorescent protein (BFP), yellow fluorescent protein (YFP), red fluorescent protein (RFP), and enzymes including luciferase and β-galactosidase.

In addition, the fusion partners, including presentation structures, may be modified, randomized, and/or matured to alter the presentation orientation of the randomized expression product. For example, determinants at the base of the loop may be modified to slightly modify the internal loop peptide tertiary structure, which maintaining the randomized amino acid sequence.

In a preferred embodiment, combinations of fusion partners are used. Thus, for example, any number of combinations of presentation structures, targeting sequences, rescue sequences, and stability sequences may be used, with or without linker sequences. Similarly, as discussed herein, the fusion partners may be associated with any component of the expression vectors described herein: they may be directly fused with either the NAM enzyme, the candidate protein, or the EAS, described below, or be separate from these components and contained within the expression vector.

In addition to sequences encoding NAM enzymes and candidate proteins, and the optional fusion partners, the nucleic acids of the invention preferably comprise an enzyme attachment sequence. By “enzyme attachment sequence” or “EAS” herein is meant selected nucleic acid sequences that mediate attachment with NAM enzymes. Such EAS nucleic acid sequences possess the specific sequence or specific chemical or structural configuration that allows for attachment of the NAM enzyme and the Eas. The EAS can comprise DNA or RNA sequences in their natural conformation, or hybrids. EASs also can comprise modified nucleic acid sequences or synthetic sequences inserted into the nucleic acid molecule of the present invention.

As will be appreciated by those in the art, the choice of the EAS will depend on the NAM enzyme, as individual NAM enzymes recognize specific sequences and thus their use is paired. Thus, suitable NAM/EAS pairs are the sequences recognized by Rep proteins (sometimes referred to herein as “Rep EASs”) and the Rep proteins, the H-1 recognition sequence and H-1, etc.

In a preferred embodiment, the EAS is double-stranded. By way of example, a suitable EAS is a double-stranded nucleic acid sequence containing specific features for interacting with corresponding NAM enzymes. For example, Rep68 and Rep78 recognize an EAS contained within an AAV ITR, the sequence of which is depicted in the Figures. In addition, these Rep proteins have been shown to recognize an ITR-like region in human chromosome 19 as well, the sequence of which is shown in the Figures.

An EAS also can comprise supercoiled DNA with which a topoisomerase interacts and forms covalent intermediate complexes. Alternatively, an EAS is a restriction enzyme site recognized by an altered restriction enzyme capable of forming covalent linkages. Finally, an EAS can comprise an RNA sequence and/or structure with which specific proteins interact and form stable complexes (see, for example, Romaniuk and Uhlenbeck, Biochemistry, 24, 423944 (1985)).

The present invention relies on the specific binding of the NAM enzyme to the EAS in order to mediate linkage of the fusion enzyme to the nucleic acid molecule. One of ordinary skill in the art will appreciate that use of an EAS consisting of a small nucleic acid sequence would result in non-specific binding of the NAM enzyme to expression vectors and the host cell genome depending on the frequency that the accessible EAS motif appears in the vector or host genome. Therefore, the EAS of the present invention is preferably comprised of a nucleic acid sequence of sufficient length such that specific fusion protein-coding nucleic acid molecule attachment results. For example, the EAS is preferably greater than five nucleotides in length. More preferably, the EAS is greater than 10 nucleotides in length, e.g., with EASs of at least 15, 20, 25, 30, 35, 40, 45 or 50 nucleotides being preferred.

Moreover, preferably the EAS is present in the host cell genome in a very limited manner, such that at most, only one or two NAM enzymes can bind per genome, e.g. no more than once in a human cell genome. In situations wherein the EAS is present many times within a host cell, e.g., a human cell genome, the probability of fusion proteins encoded by the expression vector attaching to the host cell genome and not the expression vector increases and is therefore undesirable. For instance, the bacteriophage P2 A protein recognizes a relatively short DNA recognition sequence. As such, use of the P2 A protein in mammalian cells would result in protein binding throughout the host genome, and identification of the desired nucleic acid sequence would be difficult. Thus, preferred embodiments exclude the use of P2A as a NAM enzyme.

One of ordinary skill in the art will appreciate that the NAM enzyme used in the present invention or the corresponding EAS can be manipulated in order to increase the stability of the fusion protein-nucleic acid molecule complex. Such manipulations are contemplated herein, so long as the NAM enzyme forms a covalent bond with its corresponding EAS.

In addition to the components outlined above, the nucleic acids of the invention comprise capture sequences. By “capture sequences” herein is meant nucleic acid sequences that are substantially complementary to capture probes. This idea is analogous to the use of sequences for universal arrays, sometimes referred to in the art as “zip codes”. The arraying of different capture probes in specific areas on an array combined with capture sequences specific for individual capture probes allows a pooled mixture of NAP conjugates to be added to the array, and then individual NAP conjugates will be similarly arrayed by specific hybridization to the capture probes. Thus, specific capture probe/capture sequence pairs are used. What is important in this respect is that the capture probe/capture sequence hybridization complexes are specific, e.g. an individual specific capture sequence hybridizes to a specific individual capture probe, and that the hybridization complex is stable enough under experimental conditions to allow screening.

In some cases, every pad on the array has the same capture probe sequence, and each NAP conjugate has the same capture sequence. In this embodiment, the array is used more as a general affinity capture surface, in a manner similar to phage display panning. In this embodiment, the NAP conjugates are bound to the array (which can also be a continuous surface, rather than spatially separate addresses) and test molecules added. Washing and competitive assays can be done to test for protein-protein interactions and affinity.

Regardless of whether the capture probe sequence and the capture sequence are the same or different, the capture probe/capture sequence hybridization complexes are used to form biochips comprising nucleic acids, proteins, or nucleic acid/protein chips.

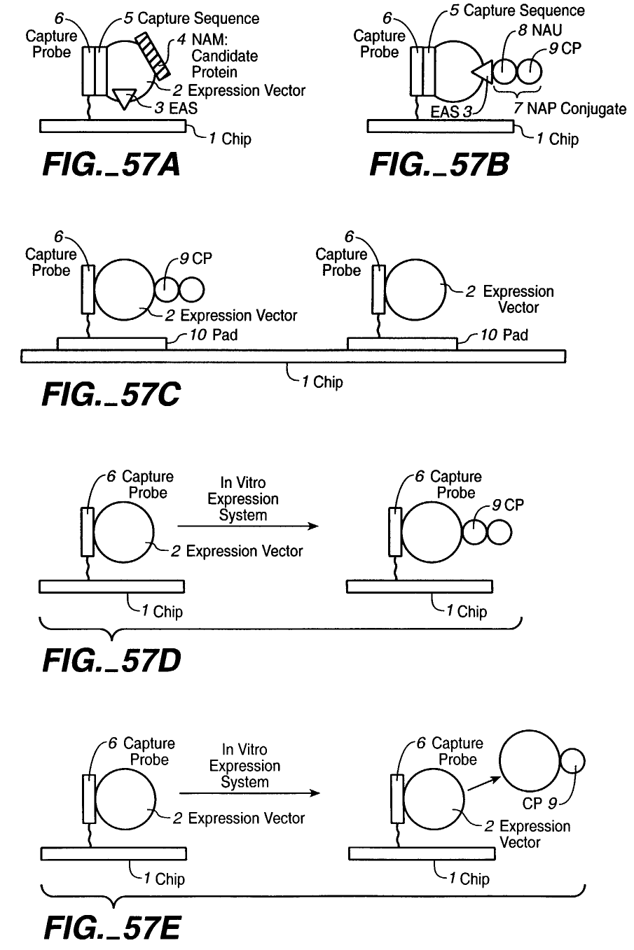

In a preferred embodiment, the capture probe/capture sequence hybridization complexes are used to form nucleic acid chips (see FIG. 57A). In this embodiment, an array of capture probes is hybridized to expression vectors encoding a NAM enzyme, a candidate protein, a capture sequence and an EAS. Preferably, each expression vector contains a fusion nucleic acid encoding a different candidate protein. As will be appreciated by those in the art, this DNA chip can be subjected to “on-chip” transcription” as described below for use in RNA profiling experiments.

In a preferred embodiment, the capture probe/capture sequence hybridization complexes are used to form “protein chips” (see FIG. 57B). In this embodiment, an array of capture probes is hybridized to nucleic acid/NAP conjugates. As will be appreciated by those in the art, the nucleic acid/NAP conjugates may be produced by culturing host cells transformed with nucleic acid or directly on the chip using in vitro expression systems. The advantage of using in vitro expression system is that the nucleic acids may be added to the chip and the chip stored as a “DNA chip”. The stored chips may then be used at a later time as a “protein chip” by performing in vitro transcription/translation as described below.

In a preferred embodiment, the capture probe/capture sequence hybridization complexes are used to form a bifunctional chip (see FIG. 57C). By “bifunctional chip” herein is meant biochip in which some of the pads have capture probes hybridized to expression vectors encoding a NAM enzyme, a candidate protein, a capture sequence and an EAS, while other pads have capture probes hybridized to nucleic acid/NAP conjugates.

Thus, in a preferred embodiment, the nucleic acids of the invention comprise a fusion nucleic acid comprising sequences encoding a NAM enzyme and a candidate protein, and an EAS and a capture sequence. These nucleic acids are preferably incorporated into an expression vector; thus providing libraries of expression vectors, sometimes referred to herein as “NAM enzyme expression vectors”.

The expression vectors may be either self-replicating extrachromosomal vectors, vectors which integrate into a host genome, or linear nucleic acids that may or may not self-replicate. Thus, specifically included within the definition of expression vectors are linear nucleic acid molecules. Expression vectors thus include plasmids, plasmid-liposome complexes, phage vectors, and viral vectors, e.g., adeno-associated virus (AAV)-based vectors, retroviral vectors, herpes simplex virus (HSV)-based vectors, and adenovirus-based vectors. The nucleic acid molecule and any of these expression vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994) Generally, these expression vectors include transcriptional and translational regulatory nucleic acid operably linked to the nucleic acid encoding the NAM protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the NAM protein, as will be appreciated by those in the art; for example, transcriptional and translational regulatory nucleic acid sequences from Bacillus are preferably used to express the NAM protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

A “promoter” is a nucleic acid sequence that directs the binding of RNA polymerase and thereby promotes RNA synthesis. Promoter sequences include constitutive and inducible promoter sequences. Exemplary constitutive promoters include, but are not limited to, the CMV immediate-early promoter, the RSV long terminal repeat, mouse mammary tumor virus (MMTV) promoters, etc. Suitable inducible promoters include, but are not limited to, the IL-8 promoter, the metallothionine inducible promoter system, the bacterial lacZYA expression system, the tetracycline expression system, and the T7 polymerases system. The promoters may be either naturally occurring promoters, hybrid or synthetic promoters. Hybrid promoters, which combine elements of more than one promoter, are also known in the art, and are useful in the present invention.

In addition, the expression vector may comprise additional elements. For example, the expression vector may have two replication systems (e.g. origins of replication), thus allowing it to be maintained in two organisms, for example in mammalian or insect cells for expression and in a prokaryotic host for cloning and amplification. Furthermore, for integrating expression vectors, which are generally not preferred in most embodiments, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors and appropriate selection and screening protocols are well known in the art and are described in e.g., Mansour et al., Cell, 51:503 (1988) and Murray, Gene Transfer and Expression Protocols, Methods in Molecular Biology, Vol. 7 (Clifton: Humana Press, 1991).

It should be noted that the compositions and methods of the present invention allow for specific chromosomal isolation. For example, since human chromosome 19 contains a Rep-binding sequence (e.g. an EAS), a NAP conjugate will be formed with chromosome 19, when the NAM enzyme is Rep. Cell lysis followed by immunoprecipitation, either using antibodies to the Rep protein itself (e.g. no candidate protein is necessary) or to a fused candidate protein or purification tag, allows the purification of the chromosome. This is a significant advance over current chromosome purification techniques. Thus, by selectively or non-selectively integrating EAS sites into chromosomes, different chromosomes may be purified.

In addition, in a preferred embodiment, the expression vector contains a selection gene to allow the selection of transformed host cells containing the expression vector, and particularly in the case of mammalian cells, ensures the stability of the vector, since cells which do not contain the vector will generally die. Selection genes are well known in the art and will vary with the host cell used. By “selection gene” herein is meant any gene which encodes a gene product that confers resistance to a selection agent. Suitable selection agents include, but are not limited to, neomycin (or its analog G418), blasticidin S, histinidol D, bleomycin, puromycin, hygromycin B, and other drugs.

In a preferred embodiment, the expression vector contains a RNA splicing sequence upstream or downstream of the gene to be expressed in order to increase the level of gene expression. See Barret et al., Nucleic Acids Res. 1991; Groos et al., Mol. Cell. Biol. 1987; and Budiman et al., Mol. Cell. Biol. 1988.

One expression vector system is a retroviral vector system such as is generally described in Mann et al., Cell, 33:153-9 (1993); Pear et al., Proc. Natl. Acad. Sci. U.S.A., 90(18):8392-6 (1993); Kitamura et al., Proc. Natl. Acad. Sci. U.S.A., 92:9146-50 (1995); Kinsella et al., Human Gene Therapy, 7:1405-13; Hofmann et al., Proc. Natl. Acad. Sci. U.S.A., 93:5185-90; Choate et al., Human Gene Therapy, 7:2247 (1996); PCT/US97/01019 and PCT/US97/01048, and references cited therein, all of which are hereby expressly incorporated by reference.

The fusion proteins of the present invention are produced by culturing a host cell transformed with nucleic acid, preferably an expression vector as outlined herein, under the appropriate conditions to induce or cause expression of the fusion protein. The conditions appropriate for fusion protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

The choice of the host cell will depend, in part, on the assay to be run; e.g. in vitro systems may allow the use of any number of procaryotic or eucaryotic organisms, while ex vivo systems preferably utilize animal cells, particularly mammalian cells with a special emphasis on human cells. Thus, appropriate host cells include yeast, bacteria, archaebacteria, fungi, and insect and animal cells, including mammalian cells and particularly human cells. The host cells may be native cells, primary cells, including those isolated from diseased tissues or organisms, cell lines (again those orginating with diseased tissues), genetically altered cells, etc. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, etc. See the ATCC cell line catalog, hereby expressly incorporated by reference.

In a preferred embodiment, the fusion proteins are expressed in mammalian cells. Mammalian expression systems are also known in the art, and include retroviral and adenoviral systems. A mammalian promoter is any DNA sequence capable of binding mammalian RNA polymerase and initiating the downstream (3′) transcription of a coding sequence for a fusion protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, NAM fusions are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of the fusion into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the fusion proteins in bacteria or other cells. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In one embodiment, NAM fusion proteins are produced in insect cells such as Sf9 cells. Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994).

In a preferred embodiment, NAM fusion proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1, 10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions. One benefit of using yeast cells is the ability to propagate the cells comprising the vectors, thus generating clonal populations.

Preferred expression vectors are shown in FIG. 49.

In a preferred embodiment, NAM fusions are produced in vitro. For example, transcription systems provided by Roche, Promega (i.e., Ribomax) and Ambion (i.e., Megascript) may be used to make RNA templates from NAM fusion vectors. Similarly, translation systems provided by Roche (i.e., linked SP6/T7 transcription/translation kit), Promega (i.e., TnT™/SP6 coupled reticulocyte system;

TnT™/SP6 coupled wheat germ extract system), Ambion (i.e., PROTEINscript II linked transcription/translation kit) may be used to make NAP conjugates from NAN fusion vectors.

In addition to the components outlined herein, including NAM enzyme-candidate protein fusions, EASs, linkers, fusion partners, etc., the expression vectors may comprise a number of additional components, including, selection genes as outlined herein (particularly including growth-promoting or growth-inhibiting functions), activatible elements, recombination signals (e.g. cre and lox sites) and labels.

In a preferred embodiment, a component of the system is a labeling component. Again, as for the fusion partners of the invention, the label may be fused to one or more of the other components, for example to the NAM fusion protein, in the case where the NAM enzyme and the candidate protein remain attached, or to either component, in the case where scission occurs, or separately, under its own promoter. In addition, as is further described below, other components of the assay systems may be labeled.

Labels can be either direct or indirect detection labels, sometimes referred to herein as “primary” and “secondary” labels. By “detection label” or “detectable label” herein is meant a moiety that allows detection. This may be a primary label or a secondary label. Accordingly, detection labels may be primary labels (i.e. directly detectable) or secondary labels (indirectly detectable).

In general, labels fall into four classes: a) isotopic labels, which may be radioactive or heavy isotopes; b) magnetic, electrical, thermal labels; c) colored or luminescent dyes or moieties; and d) binding partners. Labels can also include enzymes (horseradish peroxidase, etc.) and magnetic particles. In a preferred embodiment, the detection label is a primary label. A primary label is one that can be directly detected, such as a fluorophore.

Preferred labels include chromophores or phosphors but are preferably fluorescent dyes or moieties. Fluorophores can be either “small molecule” fluores, or proteinaceous fluores. In a preferred embodiment, particularly for labeling of target molecules, as described below, suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”), pyrene, Malacite green, stilbene, Lucifer Yellow, Cascade Blue™, Texas Red, Cy dyes (Cy3, Cy5, etc.), alexa dyes, phycoerythin, bodipy, and others described in the 6th Edition of the Molecular Probes Handbook by Richard P. Haugland, hereby expressly incorporated by reference.

In a preferred embodiment, for example when the label is attached to the fusion polypeptide or is to be expressed as a component of the expression vector, proteinaceous fluores are used. Suitable autofluorescent proteins include, but are not limited to, the green fluorescent protein (GFP) from Aequorea and variants thereof, including, but not limited to, GFP, (Chalfie, et al., Science 263(5148):802-805 (1994)); enhanced GFP (EGFP; Clontechz—Genbank Accession Number U55762)), blue fluorescent protein (BFP; Quantum Biotechnologies, Inc. 1801 de Maisonneuve Blvd. West, 8th Floor, Montreal (Quebec) Canada H3H 1J9; Stauber, R. H. Biotechniques 24(3):462-471 (1998),; Heim, R. and Tsien, R. Y. Curr. Biol. 6:178-182 (1996)), and enhanced yellow fluorescent protein (EYFP; Clontech Laboratories, Inc., 1020 East Meadow Circle, Palo Alto, Calif. 94303). In addition, there are recent reports of autofluorescent proteins from Renilla species. See WO 92/15673; WO 95/07463; WO 98/14605; WO 98/26277; WO 99/49019; U.S. Pat. Nos. 5,292,658; 5,418,155; 5,683,888; 5,741,668; 5,777,079; 5,804,387; 5,874,304; 5,876,995; and 5,925,558; all of which are expressly incorporated herein by reference.

In a preferred embodiment, the label protein is Aequorea green fluorescent protein or one of its variants; see Cody et al., Biochemistry 32:1212-1218 (1993); and Inouye and Tsuji, FEBS Lett. 341:277-280 (1994), both of which are expressly incorporated by reference herein.

In a preferred embodiment, a secondary detectable label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; enzymes such as horseradish peroxidase, alkaline phosphatases, lucifierases, etc; and cell surface markers, etc.

In a preferred embodiment, the secondary label is a binding partner pair. For example, the label may be a hapten or antigen, which will bind its binding partner. In a preferred embodiment, the binding partner can be attached to a solid support to allow separation of components containing the label and those that do not. For example, suitable binding partner pairs include, but are not limited to: antigens (such as proteins (including peptides)) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid—nucleic acid binding proteins pairs are also useful. In general, the smaller of the pair is attached to the system component for incorporation into the assay, although this is not required in all embodiments. Preferred binding partner pairs include, but are not limited to, biotin (or imino-biotin) and streptavidin, digeoxinin and Abs, etc.

In a preferred embodiment, the binding partner pair comprises a primary detection label (for example, attached to the assay component) and an antibody that will specifically bind to the primary detection label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴-10⁻⁶ M⁻¹, with less than about 10⁻⁵-10⁻⁹ M⁻¹, being preferred and less than about 10⁻⁷-10⁻⁹ M⁻¹, being particularly preferred.

In a preferred embodiment, the secondary label is a chemically modifiable moiety. In this embodiment, labels comprising reactive functional groups are incorporated into the assay component. The functional group can then be subsequently labeled with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference).

In a preferred embodiment, detection can proceed with unlabeled test molecules when a “solution binding ligands” or “soluble binding ligands” or “signalling ligands” or “signal carriers” or “label probes”or “label binding ligands” are used. In this embodiment, the soluble binding ligand carries the label and will bind to the test molecule. For example, when proteinaceous test molecules are used, they can be fused to heterologous epitope tags, which can then bind labeled antibodies to effect detection. A wide variety of epitope tags are known as outlined above.

It can be advantageous to construct the expression vector to provide further options to control attachment of the fusion enzyme to the EAS. For example, the EAS can be introduced into the nucleic acid molecule as two non-functional halves that are brought together following site-specific homologous recombination, such as that mediated by cre-lox recombination, to form a functional EAS. Likewise, the referenced cre-lox consideration could also be used to control the formation of a functional fusion enzyme. The control of cre-lox recombination is preferably mediated by introducing the recombinase gene under the control of an inducible promoter into the expression system, whether on the same nucleic acid molecule or on another expression vector.

In general, once the expression vectors of the invention are made, they follow one of two fates: they are introduced into cell-free translation systems or into cells (which are then lysed) to create libraries of nucleic acid/protein (NAP) conjugates for attachment to biochips.

In a preferred embodiment, the expression vectors are made and introduced into cell-free systems for translation, followed by the attachment of the NAP enzyme to the EAS, forming a nucleic acid/protein (NAP) conjugate. By “nucleic acid/protein conjugate” or “NAP conjugate” herein is meant a covalent attachment between the NAP enzyme and the EAS, such that the expression vector comprising the EAS is covalently attached to the NAP enzyme. Suitable cell free translation systems are known in the art. Once made, the NAP conjugates are used in assays as outlined below.

In a preferred embodiment, the expression vectors of the invention are introduced into host cells as outlined herein. By “introduced into” or grammatical equivalents herein is meant that the nucleic acids enter the cells in a manner suitable for subsequent expression of the nucleic acid. The method of introduction is largely dictated by the targeted cell type, discussed below. Exemplary methods include CaPO₄ precipitation, liposome fusion, lipofectin®, electroporation, viral infection, gene guns, etc. The candidate nucleic acids may stably integrate into the genome of the host cell (for example, with retroviral introduction, outlined herein) or may exist either transiently or stably in the cytoplasm (i.e. through the use of traditional plasmids, utilizing standard regulatory sequences, selection markers, etc.). Suitable host cells are outlined above, with eucaryotic, mammalian and human cells all preferred.

Many previously described methods involve peptide library expression in bacterial cells. Yet, it is understood in the art that translational machinery such as codon preference, protein folding machinery, and post-translational modifications of, for example, mammalian peptides, are unachievable or altered in bacterial cells, if such modifications occur at all. Peptide library screening in bacterial cells often involves expression of short amino acid sequences, which can not imitate a protein in its natural configuration. Screening of these small, sub-part sequences cannot effectively determine the function of a native protein in that the requirements for, for instance, recognition of a small ligand for its receptor, are easily satisfied by small sequences without native conformation. The complexities of tertiary structure are not accounted for, thereby easing the requirements for binding. One advantage of the present invention is the ability to express and screen unknown peptides in their native environment and in their native protein conformation. The covalent attachment of the fusion enzyme to its corresponding expression vector allows screening of peptides in organisms other than bacteria. Once introduced into a eukaryotic host cell, the nucleic acid molecule is transported into the nucleus where replication and transcription occurs. The transcription product is transferred to the cytoplasm for translation and post-translational modifications. However, the produced peptide and corresponding nucleic acid molecule must meet in order for attachment to occur, which is hindered by the compartmentalization of eukaryotic cells. NAM enzyme-EAS recognition can occur in four ways, which are merely exemplary and do not limit the present invention in any way. First, the host cells can be allowed to undergo one round of division, during which the nuclear envelope breaks down. Second, the host cells can be infected with viruses that perforate the nuclear envelope. Third, specific nuclear localization or transporting signals can be introduced into the fusion enzyme. Finally, host cell organelles can be disrupted using methods known in the art.

The end result of the above-described approaches is the transfer of the expression vector into the same environment as the fusion enzyme. The non-covalent interaction between a DNA binding protein and attachment site of previously described expression libraries would not survive the procedures required to allow linkage of the fusion protein to its expression vector in eukaryotic cells. Other DNA-protein linkages described in the art, such as those using the bacterial P2 A DNA binding peptide, require the binding peptide to remain in direct contact with its coding DNA in order for binding to occur, i.e., translation must occur proximal to the coding sequence (see, for example, Lindahl, Virology, 42, 522-533 (1970)). Such linkages are only achievable in prokaryotic systems and cannot be produced in eukaryotic cells.

Once the NAM enzyme expression vectors have been introduced into the host cells, the cells are lysed. Cell lysis is accomplished by any suitable technique, such as any of a variety of techniques known in the art (see, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994), hereby expressly incorporated by reference). Most methods of cell lysis involve exposure to chemical, enzymatic, or mechanical stress. Although the attachment of the fusion enzyme to its coding nucleic acid molecule is a covalent linkage, and can therefore withstand more varied conditions than non-covalent bonds, care should be taken to ensure that the fusion enzyme-nucleic acid molecule complexes remain intact, i.e., the fusion enzyme remains associated with the expression vector.

In a preferred embodiment, the NAP conjugate may be purified or isolated after lysis of the cells. Ideally, the lysate containing the fusion protein-nucleic acid molecule complexes is separated from a majority of the resulting cellular debris in order to facilitate interaction with the target. For example, the NAP conjugate may be isolated or purified away from some or all of the proteins and compounds with which it is normally found after expression, and thus may be substantially pure. For example, an isolated NAP conjugate is unaccompanied by at least some of the material with which it is normally associated in its natural (unpurified) state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred.

NAP conjugates may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, gel filtration, and chromatofocusing. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the NAP conjugate. In some instances no purification will be necessary.

Once made and purified if necessary, the NAP conjugates are added to biochips comprising arrays of capture probes, under conditions that allow the formation of hybridization complexes between the capture sequences of the NAP conjugates to the capture probes of the biochip. This forms the protein arrays of the invention.

In an alternative embodiment, NAP conjugates are made directly on the biochip. That is, a biochip comprising nucleic acids encoding expression vectors each comprising a different fusion nucleic acid comprising a NAM enzyme and a nucleic acid encoding a candidate protein, a capture sequence and an EAS can be made as described herein. The resulting array can then be contacted with an in vitro expression system to form fusion polypeptides. The fusion polypeptides may be linked to the expression vectors encoding it via the EAS. Alternatively, soluble fusion polypeptides may be formed.

In a preferred embodiment, a coupled transcription/translation system, such as TnT™(Promega) is used to from the fusion polypeptides of the invention. In this embodiment, the fusion polypeptide is linked to the expression vector encoding it via the EAS. Thus, protein arrays directly on the surface of the biochip are formed (FIG. 57D). Once made, these protein arrays can be used in the screening assays described below.

Alternatively, soluble fusion polypeptides may be formed (FIG. 57E).

Finally, in some embodiments, fusion nucleic acids comprising RNA encoding a NAM enzyme and a candidate protein are made using an in vitro transcription systems, such as Ribomax (Promega).

In some embodiments, stabilization of the array can be done, for example by crosslinking the hybridization complexes, for example using psoralen.

Once made, the biochips of the invention find use in a variety of applications. In a preferred embodiment, the biochips are used to screen for test molecules that bind to the candidate proteins of the chips. The test molecules in this embodiment can include a wide variety of things, including libraries of proteins, nucleic acids, lipids, carbohydrates, drugs and other small molecules, etc. In some embodiments, the target analytes comprise sets of proteins comprising different SNPs, to facilitate the identification of the role and function of different SNPs within one or more proteins.

Thus, in a preferred embodiment, the biochips are used to screen for target analytes that can bind to a candidate protein of a NAP conjugate arrayed on the biochip. By “target analyte” or “test molecule” or “target molecules” or grammatical equivalents herein is meant a molecule that is added to the biochip for testing for binding to the candidate proteins of the NAP conjugates. Test molecules as used herein describes any molecule, e.g., protein, small organic molecule, carbohydrates (including polysaccharides), polynucleotide, lipids, etc.

Test molecules encompass numerous chemical classes, though typically they are organic molecules, preferably small organic compounds having a molecular weight of more than 100 and less than about 2,500 daltons. Test molecules comprise functional groups necessary for structural interaction with proteins, particularly hydrogen bonding, and typically include at least an amine, carbonyl, hydroxyl or carboxyl group, preferably at least two of the functional chemical groups. The test molecules often comprise cyclical carbon or heterocyclic structures and/or aromatic or polyaromatic structures substituted with one or more of the above functional groups. Test molecules are also found among biomolecules including peptides, saccharides, fatty acids, steroids, purines, pyrimidines, derivatives, structural analogs or combinations thereof. Particularly preferred are proteins, candidate drugs and other small molecules, and known drugs.

Test molecules are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

Suitable test molecules include organic and inorganic molecules, including biomolecules. In a preferred embodiment, the test molecule may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

Thus, suitable target molecules encompass a wide variety of different classes, including, but not limited to, cells, viruses, proteins (particularly including enzymes, cell-surface receptors, ion channels, and transcription factors, and proteins produced by disease-causing genes or expressed during disease states), carbohydrates, fatty acids and lipids, nucleic acids, chemical moieties such as small molecules, agricultural chemicals, drugs, ions (particularly metal ions), polymers and other biomaterials. Thus for example, binding to polymers (both naturally occurring and synthetic), or other biomaterials, may be done using the methods and compositions of the invention.

In a preferred embodiment, the test molecules are proteins as defined above. In a preferred embodiment, the test molecules are naturally occurring proteins or fragments of naturally occurring proteins. Thus, for example, cellular extracts containing proteins, or random or directed digests of proteinaceous cellular extracts, may be used. In this way libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

Suitable protein test molecules include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virusy, paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lambliaY. pestis, Pseudomonas, e.g. P. aeruginosa, P. putida; Chlamydia, e.g. C. trachomatis; Bordetella, e.g. B. pertussis; Treponema, e.g. T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone, testosterone, and (4) other proteins (including α-fetoprotein, carcinoembryonic antigen CEA.

In addition, any of the biomolecules for which antibodies are tested may be tested directly as well; that is, the virus or bacterial cells, therapeutic and abused drugs, etc., may be the test molecules. In addition, one or more of the proteins listed above can be used as a candidate protein within a NAP conjugate.

In a preferred embodiment, the test molecules are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In a preferred embodiment, the test molecules are proteins derived from cDNA libraries, e.g. that are encoded by mRNA. cDNA libraries from a wide variety of different cells or tissues can be used, with cells from both eukaryotic and prokaryotic cells and cell lines being preferred. As will be appreciated by those in the art, the type of cells used to generate cDNA in the present invention can vary widely. (It should also be noted that candidate proteins can be selected from any cDNA libraries described herein).

Suitable prokaryotic cells include, but are not limited to, bacteria such as E. coli, Bacillus species, and the extremophile bacteria such as thermophiles, etc.

Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, potato, alfalfa, sunflower, etc.; and animal cells, including fish, birds and mammals. Suitable fish cells include, but are not limited to, those from species of salmon, trout, tulapia, tuna, carp, flounder, halobut, swordfish, cod and zebrafish. Suitable bird cells include, but are not limited to, those of chickens, ducks, quail, pheasants and turkeys, and other jungle foul or game birds. Suitable mammalian cells include, but are not limited to, cells from horses, cows, buffalo, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes.

As is described herein, cell types implicated in a wide variety of disease conditions are particularly useful to identify interesting protein-protein interactions. Accordingly, suitable eukaryotic cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, COS, etc. See the ATCC cell line catalog, hereby expressly incorporated by reference.

In one embodiment, the cells may be genetically engineered, that is, contain exogeneous nucleic acid.

Thus, in a preferred embodiment, the biochips are used in assays to determine protein-protein interactions, analogous to a “two hybrid” screen. In this embodiment, a library of proteinaceous test molecules, preferably cDNA derived (although random peptides can also be used) can be added to a biochip whose candidate peptides are derived from cDNA (either complete or fragmented cDNA), for example, and the interactions determined.

This embodiment can be analogous to phage display technologies, where protein-protein interactions are elucidated.

In addition, a preferred embodiment utilizes a NAP biochip and test molecules comprising NAP conjugates as well, to allow easy identification of the test molecule. This can be done as outlined herein, and in some embodiments utilizes two different selection markers (for example, different drug resistant genes); one in the surface bound NAP and one in the solution NAP. By removing both NAP conjugates from a particular address and selecting for transformants on two different antibiotics, the sequence of the candidate proteins can be elucidated. Alternatively, the solution NAP conjugate can be pulled out by identifying it via its capture sequence and sequencing it out.

In a preferred embodiment, the test molecules are nucleic acids as defined above. As described above generally for proteins, nucleic acid test molecules may be naturally occurring nucleic acids, random nucleic acids, or “biased” random nucleic acids. For example, digests of procaryotic or eukaryotic genomes may be used as is outlined above for proteins. This embodiment finds particular use in the identification and elucidation of nucleic acid/protein interactions, for example in the discovery and analysis of transcription factors. For example, biochips comprising potential transcription factor candidate proteins can be used to identify proteins that bind to DNA; then screening for small molecule drug candidates that bind to the proteins.

In a preferred embodiment, the test molecules are organic chemical moieties, a wide variety of which are available in the literature.

In a preferred embodiment, the test molecules are drugs, drug analogs or prodrugs. This is particularly useful to help elucidate the mechanism of drug action; for example, there are a wide variety of known drugs for which the targets and/or mechanism of action is unknown. By adding the drugs to biochips comprising candidate proteins, the proteins to which the drugs bind can be identified, and signaling and disease pathways can be constructed.

In a preferred embodiment, the biochips of the invention are used in SNP (single nucleotide polymorphism) analysis. There is a major effort to elucidate SNPs in different genes, particularly for genes or proteins known to be associated with disease states. However, the correlation of different bases (and the corresponding amino acid changes in the proteins) with functionality is of great interest, and a significant task. The present invention can be used to help elucidate functionality of different SNPs. In this embodiment, sets or libraries of NAP conjugates comprising candidate proteins can be made from one or more genes comprising at least one, and preferably multiple SNPs at different positions. Thus for example it is known that the BRC1 gene comprises over a thousand different SNPS. NAP conjugates comprising sets of SNPs, from one or more genes, can be constructed and tested in a variety of ways, including using proteins as the test molecules, to look for differential protein binding between different SNP proteins, or for differential binding to drugs or drug candidates. As will be appreciated by those in the art, either the SNP set can be included as NAP conjugates on the biochips, or they may be a library of test molecules added to the biochip, for example when the biochip comprises cDNA from an interesting cell. It may also be useful to put proteins comprising SNPs from the same or related disease pathways on a single biochip.

In a preferred embodiment, different patient samples can be compared for SNP analysis. That is, cDNA library-based NAP conjugates can be placed on chips, with different patient samples either on different chips or added to different chips (although as will be appreciated by those in the art, NAP conjugates from more than one patient can be placed on a single biochip as well). That is, it is possible to have the patient samples be the NAP conjugates, or cDNA derived test molecules be added to chips comprising NAP conjugates from any number of cells. For example, normal samples and samples from patients with cancer may be added to NAP conjugate chips from normal tissues, to evaluate differences in binding between normal samples and diseased samples to a particular NAP conjugate library. As for all the systems outlined herein, this may be run in reverse: the diseased samples can be incorporated as the NAP conjugates, and normal samples (and diseased samples as well) added. In addition, differential screening may be done using patient samples labeled with different labels, analogous to the “two color” nucleic acid chip analysis (see U.S. Pat. No. 5,800,992) hereby expressly incorporated by reference.

It should also be noted that this type of SNP analysis is not limited to the use of biochips; this type of analysis can be done in solution based assays, or on immobilized systems not utilizing biochips, as is generally described in PCT US00/22906 hereby expressly incorporated by reference.

In a preferred embodiment, a library of different test molecules are used. As for the candidate proteins, preferably, the library should provide a sufficiently structurally diverse population of randomized agents to effect a probabilistically sufficient range of diversity to allow binding to a particular target.

The test molecules are added to the array under conditions suitable for binding to the candidate proteins; this generally involves physiological or close to physiological conditions. Incubations may be performed at any temperature which facilitates optimal activity, typically between 4 and 40° C.

Incubation periods are selected for optimum activity, but may also be optimized to facilitate rapid high through put screening. Typically between 0.1 and 1 hour will be sufficient. Excess reagent is generally removed or washed away.

A variety of other reagents may be included in the assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for detection. Washing or rinsing the cells will be done as will be appreciated by those in the art at different times, and may include the use of filtration and centrifugation. When second labeling moieties (also referred to herein as “secondary labels”) are used, they are preferably added after excess non-bound target molecules are removed, in. order to reduce non-specific binding; however, under some circumstances, all the components may be added simultaneously.

Detection of bound test molecules can be accomplished in a wide variety of ways, as will be appreciated by those in the art. Some techniques rely on the use of detection labels, such as fluorescent labels, and others rely on unlabeled systems, for example on surface properties such as surface plasmon resonance to detect a binding event to an address on the array. In a preferred embodiment, labeling systems are used.

In general, there are two types of detection labels. By “detection label” or “detectable label” herein is meant a moiety that allows detection. This may be a primary label or a secondary label. Accordingly, detection labels may be primary labels (i.e. directly detectable) or secondary labels (indirectly detectable; this is analogous to a “sandwich” type assay).

In a preferred embodiment, the detection label is a primary label. A primary label is one that can be directly detected, such as a fluorophore. In general, labels fall into four classes: a) isotopic labels, which may be radioactive or heavy isotopes; b) magnetic, electrical, thermal labels; c) colored or luminescent dyes; and d) enzymes and other proteins that allow detection. Labels can also include enzymes (horseradish peroxidase, etc.) and magnetic particles. Preferred labels include chromophores or phosphors but are preferably fluorescent dyes. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”: see U.S. Ser. No. 09/315,584, hereby incorporated by reference), pyrene, Malacite green, stilbene, Lucifer Yellow, Cascade Blue™, Texas Red, Cy dyes (Cy3, Cy5, etc.), alexa dyes, phycoerythin, bodipy, and others described in the 6th Edition of the Molecular Probes Handbook by Richard P. Haugland, hereby expressly incorporated by reference. In some instances, fluorescent proteins such as GFP and others can be used as well.

In this embodiment, the test molecule is labeled with a primary label. As will be appreciated by those in the art, this can be done in a wide variety of ways, depending on the test molecule. In some cases, primary labels are added chemically using functional groups on the label and the test molecule. The functional group can then be subsequently labeled with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference).

In some systems, for example when the test molecule is a protein, the test molecule may be fused to a label protein such as GFP, using well known molecular biology techniques. Similarly, when the test molecule is a nucleic acid, fluorophores or other primary or secondary labels can be added to any number of the nucleotides using well known techniques.

In a preferred embodiment, a secondary detectable label is used. A secondary label is one that is indirectly detected; for example, a secondary label can bind or react with a primary label for detection, can act on an additional product to generate a primary label (e.g. enzymes), etc. Secondary labels include, but are not limited to, one of a binding partner pair; chemically modifiable moieties; nuclease inhibitors, enzymes such as horseradish peroxidase, alkaline phosphatases, lucifierases, etc.

In a preferred embodiment, the secondary label is a binding partner pair. For example, the label may be a hapten or antigen, which will bind its binding partner. For example, suitable binding partner pairs include, but are not limited to: antigens (such as proteins (including peptides) and small molecules) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid—nucleic acid binding proteins pairs are also useful. In general, the smaller of the pair is attached to the NTP for incorporation into the primer. Preferred binding partner pairs include, but are not limited to, biotin (or imino-biotin) and streptavidin, digeoxinin and Abs, and Prolinx™ reagents (see www.prolinxinc.com/ie4/home.hmtl).

In a preferred embodiment, the binding partner pair comprises an antigen and an antibody that will specifically bind to the antigen. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴-10⁻⁶ M⁻¹, with less than about 10⁻⁵to 10⁻⁹ M⁻¹ being preferred than about 10⁻⁷-10⁻⁹ M⁻¹ being particularly preferred.

In a preferred embodiment, the secondary label is a chemically modifiable moiety. In this embodiment, labels comprising reactive functional groups are incorporated into the test molecule. The functional group can then be subsequently labeled (e.g. either before or after the assay) with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference).

In general, the techniques outlined herein result in the addition of a detectable label to the test molecule, which binds to at least one of the candidate proteins of the NAP conjugates on the biochip. Fluorescent labels are preferred, and standard fluorescent detection techniques can then be used.

Once a binding event has been detected, the NAP conjugate can be identified. Since the location and sequence of each capture probe is known, the identification of a “hit” at a particular location will identify the NAP conjugate with the corresponding capture sequence. This capture sequence can be used to identify the coding region of the candidate protein. This can be done in a wide variety of ways, as will be appreciated by those in the art, including using PCR technologies. For example, using primers specific to the capture sequence and to the enzyme attachment sequence (EAS), the nucleic acid encoding the candidate protein can be amplified and sequenced. In some cases, depending on the density of the array and other factors, it can be possible to denature the capture sequence/capture probe hybridization complex and “rescue” the NAP conjugate, and sequence the vector.

In a preferred embodiment, the process may be used reiteratively. That is, the sequence of a candidate protein is used to generate more candidate proteins. For example, the sequence of the protein may be the basis of a second round of (biased) randomization, to develop agents with increased or altered activities. Alternatively, the second round of randomization may change the affinity of the agent. Furthermore, if the candidate protein is a random peptide, it may be desirable to put the identified random region of the agent into other presentation structures, or to alter the sequence of the constant region of the presentation structure, to alter the conformation/shape of the candidate protein.

The methods of using the present inventive library can involve many rounds of screenings in order to identify a nucleic acid of interest. For example, once a nucleic acid molecule is identified, the method can be repeated using a different target. Multiple libraries can be screened in parallel or sequentially and/or in combination to ensure accurate results. In addition, the method can be repeated to map pathways or metabolic processes by including an identified candidate protein as a target in subsequent rounds of screening.

In this way, the candidate protein is used to identify target molecules, i.e. the molecules with which the candidate protein interacts. As will be appreciated by those in the art, there may be primary target molecules, to which the protein binds or acts upon directly, and there may be secondary target molecules, which are part of the signalling pathway affected by the protein agent; these might be termed “validated targets”.

Once primary target molecules have been identified, secondary target molecules may be identified in the same manner, using the primary target as the “bait”. In this manner, signalling pathways may be elucidated. Similarly, protein agents specific for secondary target molecules may also be discovered, to allow a number of protein agents to act on a single pathway, for example for combination therapies.

In a preferred embodiment, the methods and compositions of the invention comprise a robotic system. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4° C. to 100° C.

In a preferred embodiment, Interchangeable pipet heads (single or multi-channel ) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some preferred embodiments, the instrumentation will include a detector, which can be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluroescence resonance energy transfer (FRET), SPR systems, luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

These instruments can fit in a sterile laminar flow or fume hood, or are enclosed, self-contained systems, for cell culture growth and transformation in multi-well plates or tubes and for hazardous operations. The living cells will be grown under controlled growth conditions, with controls for temperature, humidity, and gas for time series of the live cell assays. Automated transformation of cells and automated colony pickers will facilitate rapid screening of desired cells.

Flow cytometry or capillary electrophoresis formats can be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic workstation includes one or more heating or cooling components. Depending on the reactions and reagents, either cooling or heating may be required, which can be done using any number of known heating and cooling systems, including Peltier systems.

In a preferred embodiment, the robotic apparatus includes a central processing unit which communicates with a memory and a set of input/output devices (e.g., keyboard, mouse, monitor, printer, etc.) through a bus. The general interaction between a central processing unit, a memory, input/output devices, and a bus is known in the art. Thus, a variety of different procedures, depending on the experiments to be run, are stored in the CPU memory.

The above-described methods of screening biochips comprising fusion enzyme-nucleic acid molecule complexes for a nucleic acid encoding a desired candidate protein are merely based on the desired target property of the candidate protein. The sequence or structure of the candidate proteins does not need to be known. A significant advantage of the present invention is that no prior information about the candidate protein is needed during the screening, so long as the product of the identified coding nucleic acid sequence has biological activity, such as specific association with a targeted chemical or structural moiety. The identified nucleic acid molecule then can be used for understanding cellular processes as a result of the candidate protein's interaction with the target and, possibly, any subsequent therapeutic or toxic activity.

In one embodiment, the NAP conjugates are not attached via capture nucleic acid probes, but rather by affinity tags or antibodies, or using other methods for protein attachment.

All references cited herein are incorporated by reference.

### EXAMPLES

### Example 1

Observed differences between translation reactions using uncoupled versus coupled transcription/translation methods suggests that the higher efficiency of the coupled transcription/translation method (i.e., TnT) may be due to the transient co-localization of transcription and translation machinery required for mammalian protein translation. The mechanism for this co-localization may be similar to coupled transcription/translation in procaryotic cells. Thus, nascent proteins may be tethered to their cDNA template via polymerase and polysome complexes. A similar phenomenon, i.e., the tethering of nascent proteins via cis-linkage to their nucleic acid template, should occur on a biochip in which the nucleic acid template(s) is immobilized on the surface on the chip and in situ transcription and translation is performed.

To investigate this possibility, plasmid DNA containing nucleic acid fusions could be spotted onto the surface of a biochip in a particular pattern. For example, plasmids containing an EAS sequence and a NAM-FKBP12 or NAM-hER fusion under the control of a T7 promoter and appropriate translation signals could be spotted onto the surface of a biochip. In situ transcription and translation would be performed on the chip using a suitable coupled translation system, such as the Promega TNT™ coupled translation, for sixty minutes. Once translation is completed, the chip would be washed five times using PBS (phosphate buffered saline) buffer. Antibodies (i.e., anti-FKBP12 and anti-hER antibodies) may be used to detect the NAP conjugates linked to the EAS of the encoding plasmid vector. The presence of both plasmids may be detected simultaneously using antibodies conjugated to different fluorescent labels to distinguish between the two fusions. Analysis of the protein profile should correspond to the pattern of the encoding plasmid DNA on the chip.

We claim:

1. A composition comprising a substrate comprising an array of capture probes, a plurality of which are hybridized to an expression vector comprising:
a) a fusion nucleic acid comprising:
i) nucleic acid encoding said NAM enzyme; and
ii) nucleic acid encoding said candidate protein;
b) a capture sequence; and
c) an enzyme attachment sequence (EAS);
wherein said EAS and said NAM enzyme are covalently attached.

2. A composition according to claim 1 wherein said NAM enzyme is a Rep protein.

3. A composition comprising a substrate comprising an array of capture probes, a plurality of which are hybridized to a nucleic acid/protein (NAP) conjugate comprising:
a) a fusion polypeptide comprising:
i) a Rep protein; and
ii) a candidate protein;
b) an expression vector comprising:
i) a fusion nucleic acid comprising:
1) nucleic acid encoding said Rep protein; and
2) nucleic acid encoding said candidate protein;
ii) a capture sequence; and
iii) an enzyme attachment sequence (EAS);
wherein said EAS and said Rep protein are covalently attached.

4. A composition according to claim 1 or 3 wherein nucleic acid sequence encoding a candidate protein is derived from cDNA.

5. A composition according to claim 1 or 3 wherein nucleic acid sequence encoding a candidate protein is derived from genomic DNA.

6. A composition according to claim 1 or 3 wherein said candidate proteins include random sequences.

7. A composition according to claim 1 or 3 wherein said nucleic acids are directly fused.

8. A composition according to claim 1 or 3 wherein said nucleic acids are indirectly fused.

9. A composition according to claim 3 wherein said Rep protein is Rep68.

10. A library according to claim 3 wherein said Rep protein is Rep78.

11. A method of detecting the presence of a target analyte in a sample comprising:
a) contacting said sample with a biochip comprising:
i) a substrate comprising an array of capture probes, a plurality of which are hybridized to a nucleic acid/protein (NAP) conjugate each comprising:
1) a fusion polypeptide comprising:
A) a Rep protein; and
B) a candidate protein;
2) an expression vector comprising:
A) a fusion nucleic acid comprising:
i) nucleic acid encoding said Rep protein; and
ii) nucleic acid encoding said candidate protein;
B) a capture sequence; and
C) an enzyme attachment sequence (EAS);
wherein said EAS and said Rep protein are covalently attached, under conditions wherein said target analyte can bind to at least one of said candidate proteins to form an assay complex; and
b) detecting the presence of said target analyte on said substrate.

12. A method according to claim 11 wherein said target analyte is labeled with a fluorescent label.

13. A method according to claim 11 further comprising adding a labeled soluble binding ligand to said assay complex.

14. A method of making biochips comprising:
a) providing a substrate comprising an array of capture probes, a plurality of which are hybridized to a plurality of expression vectors each comprising:
i) a fusion nucleic acid comprising:
1) a nucleic acid encoding a NAM enzyme; and
2) a nucleic acid encoding a different candidate protein;
ii) a capture sequence; and
iii) an enzyme attachment sequence (EAS);
wherein said EAS and said NAM enzyme are covalently attached and wherein said capture sequences are hybridized to said capture probes;
b) adding an in vitro expression system to form fusion polypeptides, each comprising:
i) said (NAM) enzyme; and
ii) said different candidate protein;
c) forming a plurality of NAP conjugates on the surface of said biochip;
wherein said NAP conjugates are covalently linked to said EAS of said expression vector.

15. A method of making NAP conjugates comprising:
a) providing a substrate comprising an array of capture probes, a plurality of which are hybridized to a plurality of expression vectors each comprising:
i) a fusion nucleic acid comprising:
1) a nucleic acid encoding a NAM enzyme; and
2) a nucleic acid encoding a different candidate protein;
ii) a capture sequence; and
iii) an enzyme attachment sequence (EAS);
wherein said EAS and said NAM enzyme are covalently attached and wherein said capture sequences are hybridized to said capture probes;
b) contacting said substrate with an in vitro expression system to form a plurality of NAP conjugates each comprising:
i) said NAM enzyme;
ii) said different candidate protein; and
iii) at least a portion of said expression vector.

16. A composition comprising a substrate comprising an array of capture probes, a plurality of which are hybridized to:
a) a plurality of expression vectors each comprising:
1) a fusion nucleic acid comprising:
i) nucleic acid encoding said NAM enzyme; and
ii) nucleic acid encoding said candidate protein;
2) a capture sequence; and
3) an enzyme attachment sequence (EAS);
wherein said EAS and said NAM enzyme are covalently attached; and,
b) a plurality of nucleic acid/protein (NAP) conjugates comprising:
1) a fusion polypeptide comprising:
i) a Rep protein; and
ii) a candidate protein;
2) an expression vector comprising:
i) a fusion nucleic acid comprising:
1) nucleic acid encoding said Rep protein; and
2) nucleic acid encoding said candidate protein;
ii) a capture sequence; and
iii) an enzyme attachment sequence (EAS);
wherein said EAS and said Rep protein are covalently attached.


SEQUENCE LISTING (87 sequences)

SEQ ID NO: 1 - protein, 622 aa, adeno-associated virus 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQZ

SEQ ID NO: 2 - DNA, 1866 bp, adeno-associated virus 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 3 - protein, 621 aa, adeno-associated virus 2
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 4 - DNA, 1866 bp, adeno-associated virus 2; identical to SEQ ID NO: 2

SEQ ID NO: 5 - protein, 623 aa, adeno-associated virus 4
MPGFYEIVLKVPSDLDEHLPGISDSFVSWVAEKEWELPPDSDMDLNLIEQAPLTVAEKLQREFLVEWRRVSKAPEALFFVQFEKGDSYFHLHILVETVGVKSMVVGRYVSQIKEKLVTRIYRGVEPQLPNWFAVTKTRNGAGGGNKVVDDCYIPNYLLPKTQPELQWAWTNMDQYISACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGQNPPEDISSNRIYRILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVTHEFYVRKGGARKRPAPNDADISEPKRACPSVAQPSTSDAEAPVDYADRYQNKCSRHVGMNLMLFPCRQCERMNQNVDICFTHGVMDCAECFPVSESQPVSVVRKRTYQKLCPIHHIMGRAPEVACSACELANVDLDDCDMEQ

SEQ ID NO: 6 - DNA, 1872 bp, adeno-associated virus 4
atgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataa

SEQ ID NO: 7 - protein, 623 aa, adeno-associated virus 3B
MPGFYEIVLKVPSDLDEHLPGISNSFVNWVAEKEWELPPDSDMDPNLIEQAPLTVAEKLQREFLVEWRRVSKAPEALFFVQFEKGETYFHLHVLIETIGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGSNPPEDITKNRIYQILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFKFELTRRLDHDFGKVTKQEVKDFFRWASDHVTDVAHEFYVRKGGAKKRPASNDADVSEPKRQCTSLAQPTTSDAEAPADYADRYQNKCSRHVGMNLMLFPCKTCERMNQISNVCFTHGQRDCGECFPGMSESQPVSVVKKKTYQKLCPIHHILGRAPEIACSACDLANVDLDDCVSE

SEQ ID NO: 8 - DNA, 1875 bp, adeno-associated virus 3B
atgccggggttctacgagattgtcctgaaggtcccgagtgacctggacgagcacctgccgggcatttctaactcgtttgttaactgggtggccgagaaggaatgggagctgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttaaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacggcagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataa

SEQ ID NO: 9 - protein, 624 aa, adeno-associated virus 3
MPGFYEIVLKVPSDLDERLPGISNSFVNWVAEKEWDVPPDSDMDPNLIEQAPLTVAEKLQREFLVEWRRVSKAPEALFFVQFEKGETYFHLHVLIETIGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGSNPPEDITKNRIYQILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFEFELTRRLDHDFGKVTKQEVKDFFRWASDHVTDVAHEFYVRKGGAKKRPASNDADVSEPKRECTSLAQPTTSDAEAPADYADRYQNKCSRHVGMNLMLFPCKTCERMNQISNVCFTHGQRDCGECFPGMSESQPVSVVKKKTYQKLCPIHHILGRAPEIACSACDLANVDLDDCVSEQ

SEQ ID NO: 10 - DNA, 1875 bp, adeno-associated virus 3
atgccggggttctacgagattgtcctgaaggtcccgagtgacctggacgagcgcctgccgggcatttctaactcgtttgttaactgggtggccgagaaggaatgggacgtgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttgaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacgggagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataa

SEQ ID NO: 11 - protein, 623 aa, adeno-associated virus 1
MPGFYEIVIKVPSDLDEHLPGISDSFVSWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYEPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 12 - DNA, 1872 bp, adeno-associated virus 1
atgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataa

SEQ ID NO: 13 - protein, 623 aa, adeno-associated virus 6
MPGFYEIVIKVPSDLDEHLPGISDSFVNWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAHDLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYDPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 14 - DNA, 1872 bp, adeno-associated virus 6
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataa

SEQ ID NO: 15 - protein, 536 aa, adeno-associated virus 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 16 - DNA, 1611 bp, adeno-associated virus 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagcttttgggggcaacctcggacgagc

SEQ ID NO: 17 - protein, 536 aa, adeno-associated virus 2
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 18 - DNA, 1611 bp, adeno-associated virus 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagattggctcgaggacactctctctga

SEQ ID NO: 19 - protein, 397 aa, adeno-associated virus 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 20 - DNA, 1194 bp, adeno-associated virus 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 21 - protein, 610 aa, adeno-associated virus 5
MATFYEVIVRVPFDVEEHLPGISDSFVDWVTGQIWELPPESDLNLTLVEQPQLTVADRIRRVFLYEWNKFSKQESKFFVQFEKGSEYFHLHTLVETSGISSMVLGRYVSQIRAQLVKVVFQGIEPQINDWVAITKVKKGGANKVVDSGYIPAYLLPKVQPELQWAWTNLDEYKLAALNLEERKRLVAQFLAESSQRSQEAASQREFSADPVIKSKTSQKYMALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRYDCKCDYHAQFDNISNKCDECEYLNRGKNGCICHNVTHCQICHGIPPWEKENLSDFGDFDDANKEQ

SEQ ID NO: 22 - DNA, 1833 bp, adeno-associated virus 5
atggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaa

SEQ ID NO: 23 - protein, 312 aa, adeno-associated virus 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 24 - DNA, 939 bp, adeno-associated virus 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagcttttgggggcaacctcggacgagc

SEQ ID NO: 25 - protein, 627 aa, Barbarie duck parvovirus
MAFSRPLQISSDKFYEVIIRLPSDIDQDVPGLSLNFVEWLSTGVWEPTGIWNMEHVNLPMVTLADKIKNIFIQRWNQFNQDETDFFFQLEEGSEYIHLHCCIAQGNVRSFVLGRYMSQIKDSILRDVYEGKQVKIPDWFSITKTKRGGQNKTVTAAYILHYLIPKKQPELQWAFTNMPLFTAAALCLQKRQELLDAFQESEMNAVVQEDQASTAAPLISNRAAKNYSNLVDWLIEMGITSEKQWLTENKESYRSFQATSSNNRQVKAALENARAEMLLTKTATDYLIGKDPVLDITKNRIYQILKLNNYNPQYVGSVLCGWVKREFNKRNAIWLYGPATTGKTNIAEAIAHAVPFYGCVNWTNENFPFNDCVDKMLIWWEEGKMTNKVVESAKAILGGSAVRVDQKCKGSVCIEPTPVIITSNTDMCMIVDGNSTTMEHRIPLEERMFQIVLSHKLEGNFGKISKKEVKEFFKWANDNLVPVVSEFKVPTNEQTKLTEPVPERANEPSEPPKIWAPPTREELEEILRASPELFASVAPLPSSPDTSPKRKKTRGEYQVRCAMHSLDNSMNVFECLECERANFPEFQSLGENFCNQHGWYDCAFCNELKDDMNEIEHVFAIDDMENEQ

SEQ ID NO: 26 - DNA, 1884 bp, Barbarie duck parvovirus
atggcattttctaggcctcttcagatttcttctgacaaattctatgaagttatcatcaggctaccctcggatattgatcaagatgtgcctggtttgtctcttaactttgtagaatggctttctacgggggtctgggagcccaccggaatatggaatatggagcatgtgaatctccccatggttactctggcagacaaaatcaagaacattttcatccagagatggaaccaattcaatcaggacgaaacggatttcttctttcaattggaagaaggcagtgagtacatccatctgcattgctgtattgcccaggggaatgtccgatcttttgttctggggagatacatgtctcaaattaaagactcaattctgagagatgtgtatgaagggaaacaggtaaaaatcccggattggttttctataactaaaaccaaacggggagggcaaaataagaccgtgactgctgcttatattctgcattacctgattcctaaaaaacaaccggaattacaatgggcttttaccaatatgccccttttcactgctgctgctttatgcctccaaaagaggcaagagttactggatgcttttcaggaaagtgagatgaatgctgtagtgcaggaggatcaagcttcaactgcagctccccttatttccaacagagcagcaaagaactatagcaatctggttgattggctcattgagatgggtatcacctctgaaaaacagtggctaactgaaaataaagagagctaccggagctttcaggctacatcttcaaacaacagacaagtaaaagcagcacttgaaaatgcccgagcagaaatgctactaacaaaaactgccacagactatttgattggaaaagacccagttctggacattactaaaaatcggatctatcaaattctgaagttgaataactataaccctcaatatgtagggagcgtcctatgcggatgggtgaaaagagaattcaacaaaagaaatgccatatggctctacggacctgcgaccaccggaaagaccaacatagccgaggctattgcccatgctgtacccttctatggctgtgttaactggactaatgagaacttcccatttaatgactgcgttgataaaatgcttatatggtgggaggagggaaaaatgaccaataaagtagtggaatccgcaaaagcgatactgggggggtctgctgtacgagttgatcaaaagtgtaaggggtctgtttgtattgaacctactcctgtaataattaccagtaatactgatatgtgcatgattgtggatggaaattctactacaatggaacacagaattcctttggaggaaagaatgttccagattgttctttcccataagctggaaggaaattttggaaaaatttcaaaaaaggaggtaaaagagtttttcaaatgggccaatgataatcttgttccagtagtttctgagttcaaagtccctacgaatgaacaaaccaaacttactgagcccgttcctgaacgagcgaatgagccttccgagcctcctaagatatgggctccacctactagggaggagctagaggagatattaagagcgagccctgagctctttgcttcagttgctcctctgccttccagtccggacacatctcctaagagaaagaaaacccgtggggagtatcaggtacgctgtgctatgcacagtttagataactctatgaatgtttttgaatgcctggagtgtgaaagagctaattttcctgaatttcagagtctgggtgaaaacttttgtaatcaacatgggtggtatgattgtgcattctgtaatgaactgaaagatgacatgaatgaaattgaacatgtttttgctattgatgatatggagaatgaacaataa

SEQ ID NO: 27 - protein, 627 aa, goose parvovirus
MALSRPLQISSDKFYEVIIRLSSDIDQDVPGLSLNFVEWLSTGVWEPTGIWNMEHVNLPMVTLAEKIKNIFIQRWNQFNQDETDFFFQLEEGSEYIHLHCCIAQGNVRSFVLGRYMSQIKDSIIRDVYEGKQIKIPDWFAITKTKRGGQNKTVTAAYILHYLIPKKQPELQWAFTNMPLFTAAALCLQKRQELLDAFQESDLAAPLPDPQASTVAPLISNRAAKNYSNLVDWLIEMGITSEKQWLTENRESYRSFQATSSNNRQVKAALENARAEMLLTKTATDYLIGKDPVLDITKNRVYQILKMNNYNPQYIGSILCGWVKREFNKRNAIWLYGPATTGKTNIAEAIAHAVPFYGCVNWTNENFPFNDCVDKMLIWWEEGKMTNKVVESAKAILGGSAVRVDQKCKGSVCIEPTPVIITSNTDMCMIVDGNSTTMEHRIPLEERMFQIVLSHKLEPSFGKISKKEVREFFKWANDNLVPVVSEFKVRTNEQTNLPEPVPERANEPEEPPKIWAPPTREELEELLRASPELFSSVAPIPVTPQNSPEPKRSRNNYQVRCALHTYDNSMDVFECMECEKANFPEFQPLGENYCDEHGWYDCAICKELKNELAEIEHVFELDDAENEQ

SEQ ID NO: 28 - DNA, 1884 bp, goose parvovirus
atggcactttctaggcctcttcagatttcttctgataaattctatgaagttattattagattatcatcggatattgatcaagatgtccccggtctgtctcttaactttgtagaatggctttctaccggagtttgggagcccacgggcatctggaacatggagcatgtgaatctaccgatggtgaccttggcagagaagatcaagaacattttcatacaaagatggaatcagttcaaccaggacgaaacggacttcttctttcaactggaagaaggcagtgagtacattcatcttcattgctgtattgcccagggcaatgtacggtcttttgttctcgggagatatatgtctcagataaaagactctatcataagagatgtatatgaagggaaacaaatcaagatccccgattggtttgctattactaaaaccaagaggggaggacagaataagaccgtgactgcagcatacatactgcattaccttattcctaaaaagcaacctgaactgcaatgggcctttaccaatatgcctttattcactgctgctgctctttgtctgcaaaagcggcaagaattgctggatgcatttcaagaaagtgatttggctgcccctttacctgatcctcaagcatcaactgtggcaccgcttatttccaacagagcggcaaagaactatagcaaccttgttgattggctcattgaaatggggataacatctgagaagcaatggctcactgagaaccgagagagctacagaagctttcaagcaacttcttcaaataatagacaagtgaaagctgcactggaaaatgcccgtgctgaaatgttattgacaaagactgcaactgattacctgataggaaaagaccctgtcctggatataactaagaatagggtctatcaaattctgaaaatgaataactacaaccctcaatacataggaagtatcctgtgcggctgggtgaagagagagttcaacaaaagaaacgccatatggctctacggacctgccaccaccgggaagaccaacattgcagaagctattgcccatgctgtacccttctatggctgtgttaactggactaatgagaactttccttttaatgattgtgttgataaaatgctgatttggtgggaggagggaaaaatgactaataaggttgttgaatctgcaaaagcaattttgggagggtctgctgtccgggtagaccagaaatgtaaaggatctgtttgtattgaacctactcctgtaattattactagtaatactgatatgtgtatgattgttgatggcaactctactacaatggaacatagaataccattagaggagcgtatgtttcaaattgtcctatcacataaattggagccttcttttggaaaaatttctaaaaaagaagtcagagaatttttcaaatgggccaatgacaatctagttcctgttgtgtctgagttcaaagtccgaactaatgaacaaaccaacttgccagagcccgttcctgaacgagcgaacgagccggaggagcctcctaagatctgggctcctcctactagggaggagttagaagagcttttaagagccagcccagaattgttctcatcagtcgctccaattcctgtgactcctcagaactcccctgagcctaagagaagcaggaacaattaccaggtacgctgcgctttgcatacttatgacaattctatggatgtatttgaatgtatggaatgtgagaaagcaaactttcctgaatttcaacctctgggagaaaattattgtgatgaacatgggtggtatgattgtgctatatgtaaagagttgaaaaatgaacttgcagaaattgagcatgtgtttgagcttgatgatgctgaaaatgaacaataa

SEQ ID NO: 29 - protein, 626 aa, Muscovy duck parvovirus
MAFSRPLQISSDKFYEVIIRLPSDIDQDVPGLSLNFVEWLSTGVWEPTGIWNMEHVNLPMVTLADKIKNIFIQRWNQFNQDETDFFFQLEEGSEYIHLHAVCPGECRSFVLGRYMSQIKDSILRDVYEGKQVKIPDWFSITKTKRGGQNKTVTAAYILHYLIPKKQPELQWAFTNMPLFTAAALCLQKRQELLDAFQESEMNAVVQEDQASTAAPLISNRAAKNYSNLVDWLIEMGITSEKQWLTENKESYRSFQATSSNNRQVKAALENARAEMLLTKTATDYLIGKDPVLDITKNRIYQILKLNNYNPQYVGSVLCGWVKREFNKRNAIWLYGPATTGKTNIAEAIAHAVPFYGCVNWTNENFPFNDCVDKMLIWWEEGKMTNKVVESAKAILGGSAVRVDQKCKGSVCIEPTPVIITSNTDMCMIVDGNSTTMEHRIPLEERMFQIVLSHKLEGNFGKISKKEVKEFFKWANDNLVPVVSEFKVPTNEQTKLTEPVPERANEPSEPPKIWAPPTREELEEILRASPELFASVAPLPSSPDTSPKRKKTRGEYQVRCAMHSLDNSMNVFECLECERANFPEFQSLGENFCNQHGWYDCAFCNELKDDMNEIEHVFAIDDMENEQ

SEQ ID NO: 30 - DNA, 1881 bp, Muscovy duck parvovirus
atggcattttctaggcctcttcagatttcttctgacaaattctatgaagttatcatcaggctaccctcggatattgatcaagatgtgcctggtttgtctcttaactttgtagaatggctttctacgggggtctgggagcccaccggaatatggaatatggagcatgtgaatctccccatggttactctggcagacaaaatcaagaacattttcatccagagatggaaccaattcaatcaggacgaaacggatttcttctttcaattggaagaaggcagtgagtacatccatctgcatgctgtatgcccaggggaatgtcgatcttttgttctggggagatacatgtctcaaattaaagactcaattctgagagatgtgtatgaagggaaacaggtaaaaatcccggattggttttctataactaaaaccaaacggggagggcaaaataagaccgtgactgctgcttatattctgcattacctgattcctaaaaaacaaccggaattacaatgggcttttaccaatatgccccttttcactgctgctgctttatgcctccaaaagaggcaagagttactggatgcttttcaggaaagtgagatgaatgctgtagtgcaggaggatcaagcttcaactgcagctccccttatttccaacagagcagcaaagaactatagcaatctggttgattggctcattgagatgggtatcacctctgaaaaacagtggctaactgaaaataaagagagctaccggagctttcaggctacatcttcaaacaacagacaagtaaaagcagcacttgaaaatgcccgagcagaaatgctactaacaaaaactgccacagactatttgattggaaaagacccagttctggacattactaaaaatcggatctatcaaattctgaagttgaataactataaccctcaatatgtagggagcgtcctatgcggatgggtgaaaagagaattcaacaaaagaaatgccatatggctctacggacctgcgaccaccggaaagaccaacatagccgaggctattgcccatgctgtacccttctatggctgtgttaactggactaatgagaacttcccatttaatgactgcgttgataaaatgcttatatggtgggaggagggaaaaatgaccaataaagtagtggaatccgcaaaagcgatactgggggggtctgctgtacgagttgatcaaaagtgtaaggggtctgtttgtattgaacctactcctgtaataattaccagtaatactgatatgtgcatgattgtggatggaaattctactacaatggaacacagaattcctttggaggaaagaatgttccagattgttctttcccataagctggaaggaaattttggaaaaatttcaaaaaaggaggtaaaagagtttttcaaatgggccaatgataatcttgttccagtagtttctgagttcaaagtccctacgaatgaacaaaccaaacttactgagcccgttcctgaacgagcgaatgagccttccgagcctcctaagatatgggctccacctactagggaggagctagaggagatattaagagcgagccctgagctctttgcttcagttgctcctctgccttccagtccggacacatctcctaagagaaagaaaacccgtggggagtatcaggtacgctgtgctatgcacagtttagataactctatgaatgtttttgaatgcctggagtgtgaaagagctaattttcctgaatttcagagtctgggtgaaaacttttgtaatcaacatgggtggtatgattgtgcattctgtaatgaactgaaagatgacatgaatgaaattgaacatgtttttgctattgatgatatggagaatgaacaataa

SEQ ID NO: 31 - protein, 461 aa, goose parvovirus
RPELQWAFTNMPLFTAAALCLQKRQELLDAFQESDLAAPLPDPQASTVAPLISNRAAKNYSNLVDWLIEMGITSEKQWLTENRESYRSFQATSSNNRQVKAALENARAEMLLTKTATDYLIGKDPVLDITKNRVYQILKMNNYNPQYIGSILCGWVKREFNKRNAIWLYGPATTGKTNIAEAIAHAVPFYGCVNWTNENFPFNDCVDKMLIWWEEGKMTNKVVESAKAILGGSAVRVDQKCKGSVCIEPTPVIITSNTDMCMIVDGNSTTMEHRIPLEERMFQIVLSHKLEPSFGKISKKEVREFFKWANDNLVPVVSELKVRTNEQTNLPEPVPERANEPEEPPKIWAPPTREELEELLRASPELFSSVAPIPVTPQNSPEPKRSRNNYQVRCALHTYDNSMDVFECMECEKANFPEFQPLGENYCDEHGWYDCAICKELKNELAEIEHVFELDDAENEQ

SEQ ID NO: 32 - DNA, 1386 bp, goose parvovirus
cgacctgaactgcagtgggcctttaccaatatgcctttatttactgctgctgctctttgtctgcaaaagcggcaagaattgctggatgcatttcaagagagtgatttggctgcccctttacctgatcctcaagcatcaactgtggcaccgcttatttccaacagagcggcaaagaactatagcaaccttgttgattggctcattgaaatgggcataacatctgagaagcaatggctcactgagaaccgagagagctacagaagctttcaagcaacttcttcaaataatagacaagtgaaagctgcactggagaatgcccgtgctgaaatgctattaacaaagactgcaactgattacctgataggaaaagaccctgtcctggatataactaagaacagggtctatcaaattctgaaaatgaataactacaaccctcaatacataggaagtatcctgtgcggctgggtgaagagagagttcaacaaaagaaacgccatatggctctacggacctgccaccaccgggaagaccaacattgcagaagctattgcccatgctgtacccttctatggctgcgttaactggactaatgagaactttccttttaatgattgtgttgataagatgctgatttggtgggaggagggaaaaatgactaataaggttgttgaatctgcaaaagcaattttgggagggtctgctgtccgggtagaccagaaatgtaaaggatctgtttgtattgaacctactcctgtaattattaccagtaatactgatatgtgtatgattgttgatggcaactctactacaatggaacatagaataccattagaggagcgcatgtttcaaattgtcctatcacataaattggagccttctttcggaaaaatatctaaaaaggaagtcagagaatttttcaaatgggccaacgacaatttagttcctgttgtgtctgagctcaaagtccgaacgaatgaacaaaccaacttgccagagcccgttcctgaacgagcgaacgagccagaggagcctcctaaaatctgggctcctcctactagggaggagttagaagagcttttaagagccagcccagaattgttctcatcagttgctccaattcctgtgactcctcagaactcccctgagcctaagagaagcaggaacaattaccaggtacgctgtgctttgcatacttatgacaattctatggatgtctttgaatgtatggaatgtgagaaggcaaattttcctgaatttcaacctctgggagaaaattattgtgatgaacatgggtggtatgattgtgctatatgtaaagaattgaaaaatgaacttgcagaaattgagcatgtgtttgagcttgatgatgctgaaaatgaacaataa

SEQ ID NO: 33 - protein, 711 aa, chipmunk parvovirus
MAQACLSLSWADCFAAVIKLPCPLEEVLSNSQFWQYYVLCKDPLDWPALQVTELAHGWEVGAYCAFADALYLYLVGRLADEFSAYLLFFQLEPGVENPHIHVVAQATQLSAFNWRRILTQACHDMALGFLKPDYLGWAKNCVNIKKDKSGRILRSDWQFVETYLLPKVPLSKVWYAWTNKPEFEPIALSAAARDRLMRGNALCNQPGPGPSFGDRAEIQGPPIKKTKASDEFYTLCHWLAQEGILTEPAWRQRDLDGYVRMHTSTQGRQQVVSALAMAKNIILDSIPNSVFATKAEVVTELCFESNRCVRLLRTQGYDPVQFGCWVLRWLDRKTGKKNTIWFYGVATTGKTNLANAIAHSLPCYGCVNWTNENFPFNDAPDKCVLFWDEGRVTAKIVESVKAVLGGQDIRVDQKCKGSSFLRATPVIITSNGDMTVVRDGNTTTFAHRPAFKDRMVRLNFDVRLPNDFGLITPTEVREWLRYCKEQGDDYEFPDQMYQFPRDVVSVPAPPALPQPGPVTNAPEEEILDLLTQTNFVTQPGLSIEPAVGPEEEPDVADLGGSPAPAVSSTTESSADEDEDDDTSSSGDHRGGGGGVMGDLHASSSSFFTSSDSGLPTSVNTSDTPFSFSPVPVHHHGPPTLLPTSRPTRDLARGRPSFRQYEPLKGRCADSTTFGRPSWAAPCAVYNTAELTRRGAGVRVVKGSRPGAISGK

SEQ ID NO: 34 - DNA, 2136 bp, chipmunk parvovirus
atggctcaagcttgtctttctctgtcttgggcagattgctttgccgctgtcattaagttgccatgtcccctcgaagaggtgctgagcaacagccagttttggcaatactatgttctctgtaaagatccgcttgactggccggccttacaggtcactgagctggctcatggttgggaggtgggtgcgtactgtgcgtttgctgatgctttgtatttgtacctggtgggcagactagcagacgagtttagtgcgtacttgctgttctttcaactagaaccaggtgtggaaaatccccatattcatgttgtggcacaggccacccagttgtcggcatttaactggcgtcgcattttaactcaggcatgtcatgacatggctctggggtttttgaaacctgactacttgggctgggctaaaaattgtgtgaatattaaaaaagacaagtctggacgaattttacggtcagactggcaatttgtagaaacttacctattgcctaaagttcccctgagtaaggtctggtatgcctggactaacaagcccgaatttgagcccatagctctcagtgccgctgcgcgggacaggctgatgagaggcaacgcactttgtaatcagccgggaccggggccgtcttttggagaccgggcagaaattcagggacctcccattaaaaagactaaggcatcagatgagttttacactctctgtcactggttagctcaagagggaatattaacagagcctgcctggagacagagagatttagatggctatgtgcgtatgcacacctctactcaggggaggcagcaggtggtgtctgctcttgccatggccaaaaacatcatattggatagcattccaaactctgtgtttgccacaaaggcagaagtggtcacagaactctgttttgaaagtaaccgctgtgtgaggctcttgagaacacagggctatgacccggtacaatttggctgttgggtgttacggtggctggaccgtaaaacgggcaaaaaaaatactatttggttttatggggtcgctactactgggaaaactaatctagcaaatgcgattgcccactcacttccatgttatggctgtgtaaactggaccaatgaaaacttcccctttaatgacgcccccgacaaatgtgtattgttttgggacgagggtagagtcacggccaaaattgtggaaagtgttaaagctgtgttgggaggccaagacatcagagtggatcagaagtgtaaggggagctctttcttaagggctaccccagtcattataacaagtaatggggacatgaccgttgtgcgagatggaaataccacaaccttcgcccatcgccctgcctttaaggaccgcatggtccgcttaaattttgatgtgaggctcccaaatgactttgggcttatcacccccactgaggttcgcgagtggctgagatactgcaaggaacaaggggacgattatgagttcccagaccagatgtaccagtttccacgagatgttgtttctgttcctgctcctcctgccttgcctcagccagggccagtcacaaatgccccggaagaagagatccttgatctccttacccaaacaaacttcgtcactcaacctgggctctctattgagccggccgttggacctgaagaagaacctgatgtcgcagatcttggagggtctccagcaccagcagtcagcagcaccacagagtccagtgccgacgaggacgaggacgacgacacctcctcctctggcgaccacagaggaggaggaggaggggtcatgggagatttacacgcttcttcttcctccttctttacttccagtgactcaggactccccacttccgtcaacaccagcgacacccctttctccttcagccccgtaccagtgcaccaccacggacccccaacgcttctcccgacctcacgcccgacacgcgatctggcccgtgggcgcccgtctttccgccagtacgagccattgaaaggccggtgtgcggactcgactacgtttggtcgtccgtcttgggccgccccgtgtgcagtctacaacactgcggagctgactcgtcgtggagcaggtgtccgagttgtgaaggggtcaagaccaggtgcgatctctggaaagtga

SEQ ID NO: 35 - protein, 672 aa, pig-tailed macaque parvovirus
MEMFRGVVHVSANFINFVNDNWWCCFYQLEEDDWPRLQGWERLIAHLIVKVAGEFAVPGGSTLGLQYFLQAEHNHFDEGFHVHVVVGGPFVTPRNVCNIVETGFNKVLRELTEPTYEVSFKPAISKKGKYARDGFDFVTNYLMPKLYPNVVYSVTNFSEYEYVCNSLAYRRNMHKKALTNTADEGEGTSTNSEWGPEPKKQKTGTVRGEKFVSLVDSLIERGIFTENKWKQVDWLKEYACLSGSVAGVHQIKTALTLAISKCNSPEYLCELLTRPSTINFNIKENRICKIFLQNDYDPLYAGKVFLAWLGKELGKRNTIWLFGPPTTGKTNIAMSLATAVPSYGMVNWNNENFPFNDVPHKSIILWDEGLIKSTVVEAAKAILGGQNCRVDQKNKGSVEVQGTPVLITSNNDMTRVVSGNTVTLIHQRALKDRMVEFDLTVRCSNALGLIPAEECKQWLFWSQHTPCDVFSRWKEVCEFVAWKSDRTGICYDFSENEDLPGTQTPLLNSPVTSKTSALKKTIAALATAAVGTLQTSLTNNNWESSEDSGSPPRSSTPLASPERGEVPPGQQWELNTSVNSVNALNWPMYTVDWVWGSKAQRPVCCLEHDTESSVHCSLCLSLEVLPMLIENSINQPDVIRCSAHAECTNPFDVLTCKKCRELSALWSFVKYD

SEQ ID NO: 36 - DNA, 2019 bp, pig-tailed macaque parvovirus
atggaaatgtttcggggtgttgtacatgtttctgctaactttattaactttgttaacgataattggtggtgttgtttttaccagttagaggaagatgactggccgcggctgcaaggctgggaaagacttatagctcacttaattgttaaagtagcaggagaatttgctgttccgggaggcagtactttagggctgcaatattttttacaagctgaacataaccactttgatgagggatttcatgtgcatgtagtagttgggggaccgtttgttactcccaggaatgtgtgtaatattgtagaaacaggctttaacaaagttttgagggaacttacagagcctacttatgaggtgtcttttaagcctgccatttctaagaaaggaaagtatgctagagatggatttgactttgtaacaaactatttaatgccaaaactgtatcctaatgttgtttactctgttacaaatttttcagagtatgagtatgtatgtaattctttagcttacagaaggaacatgcataaaaaagctttaacaaatactgcagatgaaggtgagggcaccagtacaaattcagagtggggaccagaaccaaaaaaacagaaaactggtaccgtgcgaggagaaaagtttgttagtttggttgactctttaatagagcgtggcatatttacagaaaacaagtggaagcaggtagattggcttaaagagtatgcctgtctcagtggaagtgtagcaggagtgcaccagattaaaacagctttaactttagctatttctaaatgtaattctccagaatatttgtgtgaattgttaactagacccagtactattaattttaacatcaaagaaaacagaatttgtaagatatttttacagaatgattatgatcctctgtatgctggtaaagtttttttagcttggcttggtaaagagttgggaaagcgtaataccatttggctttttggaccgcctactactggtaaaacaaatatagctatgagtcttgccactgcagtacccagttatggtatggttaattggaataatgaaaactttccttttaacgatgtgccgcataaatctattattttgtgggatgagggacttattaaaagtactgttgtggaagccgcaaaagccattttaggagggcaaaattgcagagtggatcaaaaaaataagggcagtgtagaagttcagggcactcccgttctgatcactagcaacaatgacatgactcgcgtggtgtcaggcaacactgttacgcttatccatcagagggcgctaaaggatcgcatggttgagtttgacttgactgtgagatgctctaatgcccttggattaattcccgctgaggaatgtaagcagtggttgttctggtcacagcatactccttgtgatgttttctcaaggtggaaggaagtctgtgagtttgttgcttggaaaagtgacagaacagggatttgctatgacttctcagaaaacgaagatcttccggggactcagacccctctgctgaacagcccagtgacctcgaagacatcagcattgaagaaaacgatagcggcattagcaactgcagcggttggaacattacagacctccctcacaaacaacaactgggagtcctctgaggatagcggttccccgccccgcagcagcaccccacttgcatctcctgagcgaggcgaagttccccccggacagcagtgggaactgaacacctcagtaaactctgtaaatgctttaaactggcctatgtatacagtggattgggtttggggatctaaggctcaaagacctgtgtgttgcttagagcatgatacagaaagttcagtgcattgttctttgtgcttaagtttagaggtgttgcctatgttaattgaaaacagtattaaccagcccgatgtaattaggtgctctgctcatgctgagtgtactaatccttttgatgtgcttacctgtaagaaatgtcgagagctgagtgcactgtggagttttgttaagtatgactga

SEQ ID NO: 37 - protein, 687 aa, Simian parvovirus
MEMYRGVIQVNANFTDFANDNWWCCFFQLDVDDWPELRGPERLMAHYICKVAALLDTPSGPFLGCKYFLQVEGNHFDNGFHIHVVIGGPFLTPRNVCSAVEGGFNKVLADFTSPTITVQFKPAVSKKGKYHRDGFDFVTYYLMPKLYPNVIYSVTNLEEYQYVCNSLCYRRTMHKRQQPCNGGSVEQSSVSLYSDGEPANKKSKVVTVRGEKFCSLVDSLIERNIFNENKWKETDFKEYAALSASVAGVHQIKTALTLAVSKCNSPAYLGEILTRPNTINFNIRENRIANIFLSNNYCPLYAGKMFLAWVQKQLGKRNTIWLFGPPSTGKTNIAMSLASAVPTYGMVNWNNENFPFNDVPYKSIILWDEGLIKSTVVEAAKSILGGQPCRVDQKNKGSVEVSGTPVLITSNSDMTRVVCGNTVTLVHQRALKDRMVRFDLTVRCSNALGLIPADEAKQWLWWAQNNACDAFTQWHLSSDHVAWKVDRTTLCHDFQSEPEPDSELPSSGESVESFDRSDLSTSWLDVQDQSSSPENSDVEWDIADLLSNEHWIDDLQEDSCSPPRCSTPVAVAEPVEVPTGTGGGLKWEKNYSVHDTNELRWPMFSVDWVWGTNVKRPVCCLEHDKEFGVHCSLCLSLEVLPMLIEKSILVPDTLRCSAHGDCTNPFDVLTCKKCRDLSGLMSFLEHE

SEQ ID NO: 38 - DNA, 2064 bp, Simian parvovirus
atggagatgtatagaggagttattcaggtaaatgctaactttactgactttgctaacgataactggtggtgctgcttttttcagttagatgtagatgactggccggagcttagaggacccgagaggcttatggctcactacatttgtaaagtggctgctttactggacaccccctctgggccttttttgggttgcaagtattttttgcaagtggagggcaaccattttgataatgggtttcacattcatgtggtgattgggggaccatttctaactcctagaaatgtgtgttctgctgtggaagggggttttaacaaagtgttagcagactttacaagccctactatcactgttcagtttaaacctgctgttagtaaaaaggggaaatatcatagagatggctttgactttgtaacttactatttaatgccaaaactgtaccctaatgttatttacagtgtaactaacctagaagaataccagtatgtatgtaattctctctgttataggagaacaatgcataaaaggcaacaaccatgtaatggggggtctgttgaacagtccagtgtttctttgtattctgatggagaacctgcaaacaagaaaagcaaggttgtaactgttagaggggagaaattctgctctttggtagattcacttatagaaagaaatatatttaatgaaaacaaatggaaagaaacagactttaaggagtatgctgccttaagtgcttctgtagcaggagttcaccaaattaaaactgctctcactcttgcagtgtcaaagtgtaactctccagcttatctaggagaaattttaactagacctaacactataaattttaacattagagaaaacagaattgctaacatttttttaagtaacaactattgccctctgtatgctgggaaaatgtttttagcttgggtgcagaaacagcttggtaaaaggaatactatttggctgtttggtcctcccagtactggtaaaactaacattgcaatgagtttggcctctgctgttccaacatatggcatggtaaactggaacaatgaaaattttccgtttaatgatgtaccttataaaagcattattttgtgggacgagggactaataaagtccacggttgttgaagcagcaaaaagtattttaggaggtcagccatgtagagttgatcagaaaaataagggcagcgtggaagtcagtggcactcctgtgctcattaccagcaacagtgacatgactagagtggtgtgcggtaacactgtgacccttgtccatcagcgagctttgaaggatcgcatggttcgatttgatctgactgtgagatgctctaatgctctgggattaatccctgctgatgaggccaagcagtggctttggtgggcacagaataacgcgtgtgacgcctttactcaatggcatctgtctagtgatcacgttgcttggaaagtggaccgtacaacgctgtgtcatgacttccagagcgagccggagccagacagcgaactccctagtagcggggagtcagttgagagctttgacagaagcgacctctcaacctcctggcttgacgtccaagatcagtcaagcagtcctgaaaactctgatgtcgagtgggacatcgcagacctcctctcaaacgagcactggatcgacgacctgcaagaagatagctgttccccgccccgctgcagcaccccagtggcagtggctgagccagtcgaagttcccaccggaaccggaggaggactgaagtgggaaaaaaactattctgttcatgatactaatgaactgagatggcctatgttttctgttgattgggtgtggggtacaaatgttaaacgtccagtgtgctgtttagagcacgataaggagtttggtgtgcattgcagtttgtgtttgtctttggaggttttgcctatgcttattgaaaaaagcattctggtaccagacactctaagatgttctgctcatggtgattgtactaatccttttgacgtgcttacgtgtaagaaatgccgagatctgagtggtttaatgagctttttagagcatgagtga

SEQ ID NO: 39 - protein, 683 aa, Rhesus macaque parvovirus
MDMFRGVIQLTANITDFANDSWWCSFLQLDSDDWPELRGVERLVAIFICKVAAVLDNPSGTSLGCKYFLQAEGNHYDAGFHVHIVIGGPFINARNVCNAVETTFNKVLGDLTDPSMSVQFKPAVSKKGEYYRDGFDFVTNYLMPKLYPNVIYSVTNLEEYQYVCNSLCYRKNMHKQHMVSTVDASSSSFMNDMYEPATKRSKSCTVKGEKFRNLVDSLIERNIFSESKWKEVDFNEFARLSASVAGVHQIKTAITLAVSKCNSPDYLFQILTRPSTIHFNIKENRIAQIFLNNNYCPLYAGEVFLFWIQKQLGKRNTVWLYGPPSTGKTNVAMSLASAVPTYGMVNWNNENFPFNDVPYKSLILWDEGLIKSTVVEAAKSILGGQPCRVDQKNKGSVEVTGTPVLITSNSDMTRVVWYTVTLVHQRALKDRMVRFDLTVRCSNALGLIPADEAKQWLWWAQSQPCDAFTQWHQVSEHVAWKADRTGLFHDFSTKPEQESNAKSSGKSNDSFAGSDLANLSWLDVEDTSSSSESDLSGDIAELVSNDNWLQSGCPPTRCSTPVTVVEPKQVSPGTGGGLTKWEKNYSVHQENELAWPMFSVDWVWGSHVKRPVCCVEHDKDLVLPHCNLCLSLEVLPMLIEKSINVPDTLRCSAHGDCTNPFDVLTCKKCRDLSGLMSFLEHDQ

SEQ ID NO: 40 - DNA, 2052 bp, Rhesus macaque parvovirus
atggacatgttccggggagttattcaactgactgctaacattactgactttgctaacgatagctggtggtgtagctttttgcagttagattcagatgactggccggagctgagaggtgtcgagagactagttgctatttttatttgtaaagtagctgctgtattagacaacccctctggtacatctcttggctgtaaatattttttgcaggcagagggtaatcattatgatgctggttttcatgtgcatattgttattgggggacctttcattaatgctagaaatgtatgtaatgctgttgaaactacttttaacaaggtgctgggagatcttacggatccttctatgtctgtacaatttaaacctgctgtaagcaaaaagggagagtattacagagatggttttgactttgtgactaactacttaatgccaaaactgtatcctaatgttatttactctgtaacaaacctagaagagtaccagtatgtgtgtaattcactgtgttatagaaagaacatgcataagcaacatatggtgtctactgtagatgccagtagttctagttttatgaatgatatgtatgaaccagctacaaaaagaagtaaaagctgtacagtaaaaggagagaaatttcgtaatttagtagacagtctcattgagagaaatatttttagtgaaagtaaatggaaagaagttgattttaatgagtttgctaggcttagcgcctctgtggcaggagttcatcaaattaaaacagccattactcttgcagtgtcaaagtgtaattcaccagactatctgtttcaaattttaactagacccagtactattcattttaatattaaagaaaacaggattgctcagatctttttaaacaacaactactgtccactgtatgctggagaagtattcctcttttggattcaaaagcaattaggaaaaagaaacactgtgtggttgtatgggcctcctagtactggcaaaacaaatgtggctatgagcttagcgtctgcagtgcctacttatggcatggttaactggaataatgaaaactttccatttaatgatgtgccttataaaagtttaatactgtgggacgaagggcttattaaaagtacagttgtagaggcagcaaaaagtattctgggaggtcaaccatgtagggttgatcaaaagaataaaggcagtgtagaagtcacaggcactcctgttcttattaccagtaacagtgacatgaccagagtggtgtggtatacggtgactttagtgcatcagcgagcgttgaaggatcgcatggttcggtttgacctgactgtgagatgctctaatgctctgggattaattcccgctgatgaagccaagcagtggctgtggtgggcacagagtcagccgtgtgatgcatttacccaatggcaccaggtcagtgagcacgttgcttggaaggcggaccgtacaggcttgttccatgacttcagtacaaagccggagcaggagtcaaacgcaaagtcaagcggaaaatcaaatgactcctttgcaggaagcgacctcgcaaatctctcctggcttgacgttgaagatacctcgagctcttcggagtctgatctcagcggggacattgcagaactcgtctccaacgacaactggctccagagtggctgtcccccgacccggtgcagcaccccagttacagtggttgagccaaagcaagtttcccccggaaccggaggaggattaacaaagtgggaaaaaaattattcagttcatcaagaaaatgagctagcatggcctatgtttagtgtagactgggtgtggggttctcatgtaaaacgccctgtgtgctgtgtagagcatgataaggaccttgtactgcctcattgtaatttgtgcttgtctctcgaagtgttgcctatgttaattgagaaaagtattaatgttccagatactttgcgatgttcagctcatggtgattgtactaatccatttgatgttttaacttgtaagaagtgtagagatctcagtggccttatgagttttttagaacatgaccagtag

SEQ ID NO: 41 - protein, 671 aa, B19 virus
MELFRGVLQVSSNVLDCANDNWWCSLLDLDTSDWEPLTHTNRLMAIYLSSVASKLDFTGGPLAGCLYFFQVECNKFEEGYHIHVVIGGPGLNPRNLTVCVEGLFNNVLYHLVTENVKLKFLPGMTTKGKYFRDGEQFIENYLMKKIPLNVVWCVTNIDGYIDTCISATFRRGACHAKKPRITTAINDTSSDAGESSGTGAEVVPINGKGTKASIKFQTMVNWLCENRVFTEDKWKLVDFNQYTLLSSSHSGSFQIQSALKLAIYKATNLVPTSTFLLHTDFEQVMCIKDNKIVKLLLCQNYDPLLVGQHVLKWIDKKCGKKNTLWFYGPPSTGKTNLAMAIAKSVPVYGMVNWNNENFPFNDVAGKSLVVWDEGIIKSTIVEAAKAILGGQPTRVDQKMRGSVAVPGVPVVITSNGDITFVVSGNTTTTVHAKALKERMVKLNFTVRCSPDMGLLTEADVQQWLTWCNAQSWDHYENWAINYTFDFPGINADALHPDLQTTPIVTDTSISSSGGESSEELSESSFFNLITPGAWNTETPRSSTPIPGTSSGESFVGSSVSSEVVAASWEEAFYTPLADQFRELLVGVDYVWDGVRGLPVCCVQHINNSGGGLGLCPHCINVGAWYNGWKFREFTPDLVRCSCHVGASNPFSVLTCKKCAYLSGLQSFVDYE

SEQ ID NO: 42 - DNA, 2016 bp, B19 virus
atggagctatttagaggggtgcttcaagtttcttctaatgttctggactgtgctaacgataactggtggtgctctttactggatttagacacttctgactgggaaccactaactcatactaacagactaatggcaatatacttaagcagtgtggcttctaagcttgactttaccggggggccactagcggggtgcttgtacttttttcaagtagaatgtaacaaatttgaagaaggctatcatattcatgtggttattggggggccagggttaaaccccagaaacctcacagtgtgtgtagaggggttatttaataatgtactttatcaccttgtaactgaaaatgtaaagctaaaatttttgccaggaatgactacaaaaggcaaatactttagagatggagagcagtttatagaaaactatttaatgaaaaaaatacctttaaatgttgtatggtgtgttactaatattgatggatatatagatacctgtatttctgctacttttagaaggggagcttgccatgccaagaaaccccgcattaccacagccataaatgacactagtagtgatgctggggagtctagcggcacaggggcagaggttgtgccaattaatgggaagggaactaaggctagcataaagtttcaaactatggtaaactggttgtgtgaaaacagagtgtttacagaggataagtggaaactagttgactttaaccagtacactttactaagcagtagtcacagtggaagttttcaaattcaaagtgcactaaaactagcaatttataaagcaactaatttagtgcctacaagcacatttctattgcatacagactttgagcaggttatgtgtattaaagacaataaaattgttaaattgttactttgtcaaaactatgaccccctattagtggggcagcatgtgttaaagtggattgataaaaaatgtggcaagaaaaatacactgtggttttatgggccgccaagtacaggaaaaacaaacttggcaatggccattgctaaaagtgttccagtatatggcatggttaactggaataatgaaaactttccatttaatgatgtagcagggaaaagcttggtggtctgggatgaaggtattattaagtctacaattgtagaagctgcaaaagccattttaggcgggcaacccaccagggtagatcaaaaaatgcgtggaagtgtagctgtgcctggagtacctgtggttataaccagcaatggtgacattacttttgttgtaagcgggaacactacaacaactgtacatgctaaagccttaaaagagcgaatggtaaagttaaactttactgtaagatgcagccctgacatggggttactaacagaggctgatgtacaacagtggcttacatggtgtaatgcacaaagctgggaccactatgaaaactgggcaataaactacacttttgatttccctggaattaatgcagatgccctccacccagacctccaaaccaccccaattgtcacagacaccagtatcagcagcagtggtggtgaaagctctgaagaactcagtgaaagcagcttttttaacctcatcaccccaggcgcctggaacactgaaaccccgcgctctagtacgcccatccccgggaccagttcaggagaatcatttgtcggaagctcagtttcctccgaagttgtagctgcatcgtgggaagaagccttctacacacctttggcagaccagtttcgtgaactgttagttggggttgattatgtgtgggacggtgtaaggggtttacctgtgtgttgtgtgcaacatattaacaatagtgggggaggcttgggactttgtccccattgcattaatgtaggggcttggtataatggatggaaatttcgagaatttaccccagatttggtgcggtgtagctgccatgtgggagcttctaatcccttttctgtgctaacctgcaaaaaatgtgcttacctgtctggattgcaaagctttgtagattatgagtaa

SEQ ID NO: 43 - protein, 671 aa, Erythrovirus B19
MELFRGVLQVSSNVLDCANDNWWCSLLDLDTSDWEPLTHTNRLMAIYLSSVASKLDFTGGPLAGCLYFFQVECNKFEEGYHIHVVIGGPGLNPRNLTMCVEGLFNNVLYHLVTENVKLKFLPGMTTKGKYFRDGEQFIENYLIKKIPLNVVWCVTNIDGYIDTCISATFRRGACHAKKPRITTAINDTSSDAGESSGTGAEVVPFNGKGTKASIKFQTMVNWLCENRVFTEDKWKLVDFNQYTLLSSSHSGSFQIQSALKLAIYKATNLVPTSTFLLHTDFEQVMCIKDNKIVKLLLCQNYDPLLVGQHVLKWIDKKCGKKNTLWFYGPPSTGKTNLAMAIAKSVPVYGMVNWNNENFPFNDVAGKSLVVWDEGIIKSTIVEAAKAILGGQPTRVDQKMRGSVAVPGVPVVITSNGDITFVVSGNTTTTVHAKALKERMVKLNFTVRCSPDMGLLTEADVQQWLTWCNAQSWDHYENWAINYTFDFPGINADALHPDLQTTPIVTDTSISSSGGESSEELSESSFLNLITPGAWNTETPRSSTPIPGTSSGESFVGSPVSSEVVAASWEEAFYTPLADQFRELLVGVDYVWDGVRGLPVCCVQHINNSGGGLGLCPHCINVGAWYNGWKFREFTPDLVRCSCHVGASNPFSVLTCKKCAYLSGLQSFVDYE

SEQ ID NO: 44 - DNA, 2016 bp, Erythrovirus B19
atggagctatttagaggggtgcttcaagtttcttctaatgttctggactgtgctaacgataactggtggtgctctttactggatttagacacttctgactgggaaccactaactcatactaacagactaatggcaatatacttaagcagtgtggcttctaagcttgactttaccggggggccactagcagggtgcttgtacttttttcaagtagaatgtaacaaatttgaagaaggctatcatattcatgtggttattggggggccagggttaaaccccagaaacctcactatgtgtgtagaggggttatttaataatgtactttatcaccttgtaactgaaaatgtgaagctaaaatttttgccaggaatgactacaaaagggaaatactttagagatggagagcagtttatagaaaactatttaataaaaaaaatacctttaaatgttgtatggtgtgttactaatattgatggatatatagatacctgtatttctgctacttttagaaggggagcttgccatgccaagaaaccccgcattaccacagccataaatgatactagtagtgatgctggggagtctagcggcacaggggcagaggttgtgccatttaatgggaagggaactaaggctagcataaagtttcaaactatggtaaactggttgtgtgaaaacagagtgtttacagaggataagtggaaactagttgactttaaccagtacactttactaagcagtagtcacagtggaagttttcaaattcaaagtgcactaaaactagcaatttataaagcaactaatttagtgcctactagcacatttttattgcatacagactttgagcaggttatgtgtattaaagacaataaaattgttaaattgttactttgtcaaaactatgaccccctattggtggggcagcatgtgttaaagtggattgataaaaaatgtggcaaaaaaaatacactgtggttttatgggccgccaagtacaggaaaaacaaacttggcaatggccattgctaaaagtgttccagtatatggcatggttaattggaataatgaaaactttccatttaatgatgtagcagggaaaagcttggtggtctgggatgaaggtattattaagtctacaattgtagaagctgcaaaagccattttaggcgggcaacccaccagggtagatcaaaaaatgcgtggaagtgtagctgtgcctggagtacctgtggttataaccagcaatggtgacattacttttgttgtaagcgggaacactacaacaactgtacatgctaaagccttaaaagagcgcatggtaaagttaaactttactgtaagatgcagccctgacatggggttactaacagaggctgatgtacaacagtggcttacatggtgtaatgcacaaagctgggaccactatgaaaactgggcaataaactacacttttgatttccctggaattaatgcagatgccctccacccagacctccaaaccaccccaattgtcacagacaccagtatcagcagcagtggtggtgaaagctctgaagaactcagtgaaagcagctttcttaacctcatcaccccaggcgcctggaacactgaaaccccgcgctctagtacgcccatccccgggaccagttcaggagaatcatttgtcggaagcccagtttcctccgaagttgtagctgcatcgtgggaagaagctttctacacacctttggcagaccagtttcgtgaactgttagttggggttgattatgtgtgggacggtgtaaggggtttacctgtgtgttgtgtgcaacatattaacaatagtgggggaggcttgggactttgtccccattgcattaatgtaggggcttggtataatggatggaaatttcgagaatttaccccagatttggtgcggtgtagctgccatgtgggagcttctaatcccttttctgtgctaacctgcaaaaaatgtgcttacctgtctggattgcaaagctttgtagattatgagtaa

SEQ ID NO: 45 - protein, 490 aa, Human herpesvirus 6B
MFSIINPSDDFWTKDKYIMLTIKGPVEWEAEIPGISTDFFCKFSNVPVPHFRDMHSPGAPDIKWITACTKMIDVILNYWNNKTAVPTPAKWYAQAENKAGRPSLTLLIALDGIPTATIGKHTTEIRGVLIKDFFDGNAPKIDDWCTYAKTKKNGGGTQVFSLSYIPFALLQIIRPQFQWAWTNINELGDVCDEIHRKHIISHFNKKPNVKLMLFPKDGTNRISLKSKFLGTIEWLSDLGIVTEDAWIRRDVRSYMQLLTLTHGDVLIHRALSISKKRIRATRKAIDFIAHIDTDFEIYENPVYQLFCLQSFDPILAGTILYQWLSHRRGKKNTVSFIGPPGCGKSMLTGAILENIPLHGILHGSLNTKNLRAYGQVLVLWWKDISINFENFNIIKSLLGGQKIIFPINENDHVQIGPCPIIATSCVDIRSMVHSNIHKINLSQRVYNFTFDKVIPRNFPVIQKDDINQFLFWARNRSINCFIDYTVPKIL

SEQ ID NO: 46 - DNA, 1473 bp, Human herpesvirus 6B
atgttttccataataaatccaagtgatgatttttggactaaggacaaatatatcatgttgactatcaaaggccccgtggagtgggaggcagaaatccctggaatatctacggattttttttgcaaattctctaacgtgcccgtgccacattttagagatatgcactcaccgggagcgcccgatattaaatggataactgcatgtaccaaaatgatcgatgtcatactcaattactggaataataaaactgccgtccccacccctgcaaagtggtacgctcaagcggagaataaagctggcagaccctccttaacattattgatagctttagatggaattcccaccgcaacgataggaaaacacacaacggaaatcaggggtgtattaattaaagatttcttcgacgggaacgcccctaaaatagatgattggtgcacgtatgccaaaacaaagaaaaatggtggcggaacccaggtcttcagtctaagttatatcccctttgcccttcttcaaattattagaccacagttccaatgggcatggacaaatattaacgaactgggagacgtatgcgatgaaatacatcgaaaacacatcatatcccatttcaataaaaaacctaatgttaaacttatgctgtttccaaaggatgggaccaacagaatatctttaaaatctaaatttctgggaaccatcgaatggctgtctgatcttggaatagtcacggaagacgcgtggatacgaagagacgttagatcatacatgcaattattgacactaacacacggggacgtgctaattcatagggctctatctatatctaaaaaaagaataagagcaactagaaaagctatcgattttatagcgcacatagacactgactttgaaatctatgaaaacccggtttaccagttgttctgtctgcagtcttttgaccctatattagcaggaaccatattatatcagtggctaagccacagaagagggaaaaaaaacaccgttagttttattggtccacccggatgtggaaaatcgatgttaacgggagccattcttgaaaatatcccgttacatggaatattacacggatctttgaatactaaaaatttaagagcttacggacaggttttagtcttgtggtggaaagacataagtatcaactttgaaaattttaatattataaaatccctccttgggggtcaaaaaataatattcccaattaatgaaaacgaccacgtacagataggaccgtgtcccatcatagccacatcttgcgttgatatacgctcgatggtacattcaaatatccacaaaataaatctatcacagagggtatataattttacatttgataaagttatccctcgcaattttcctgtaattcagaaagacgacataaatcaatttctgttctgggccagaaaccgttctataaattgttttattgactacacggttccaaaaattttataa

SEQ ID NO: 47 - DNA, 63 bp, unidentified adenovirus
ttggccactccctctctgcgcgctcgctcgctcactgaggccgggcgaccaaaggtcgcccga

SEQ ID NO: 48 - DNA, 43 bp, Homo sapiens
ggcggttggggctcggcgctcgctcgctcgctgggcgggcggg

SEQ ID NO: 49 - protein, 20 aa, Artificial sequence
  Consensus sequence for SH-3 domain binding protein
  MISC_FEATURE (3)..(7): “Xaa” at positions 3 to 7 is a randomized residue which can be hydrophobically biased
  MISC_FEATURE (13)..(16): “Xaa” at positions 13 and 15-16 indicates a bias toward a hydrophobic residue (i.e. Val, Ala, Gly, Leu, Pro, Arg).
MGXXXXXRPLPPXPXXGGPP

SEQ ID NO: 50 - DNA, 63 bp, Artificial sequence
  Oligonucleotide consensus sequence for SH-3 domain binding protein
  misc_feature (7)..(20): “n” at positions 7-8, 10-11, 13-14, 16-17, and 19-20 can be any base.
atgggcnnknnknnknnknnkagacctctgcctccasbkgggsbksbkggaggcccaccttaa

SEQ ID NO: 51 - protein, 5 aa, Artificial sequence
  linker consensus sequence
GSGGS

SEQ ID NO: 52 - protein, 4 aa, Artificial sequence
  linker consensus sequence
GGGS

SEQ ID NO: 53 - protein, 61 aa, Artificial sequence
  coiled-coil presentation structure
MGCAALESEVSALESEVASLESEVAALGRGDMPLAAVKSKLSAVKSKLASVKSKLAACGPP

SEQ ID NO: 54 - protein, 6 aa, Artificial sequence
  loop structure of coiled-coil presentation structure
GRGDMP

SEQ ID NO: 55 - protein, 69 aa, Artificial sequence
  minibody presentation structure
MGRNSQATSGFTFSHFYMEWVRGGEYIAASRHKHNKYTTEYSASVKGRYIVSRDTSQSILYLQKKKGPP

SEQ ID NO: 56 - protein, 7 aa, Simian virus 40
PKKKRKV

SEQ ID NO: 57 - protein, 6 aa, Homo sapiens
ARRRRP

SEQ ID NO: 58 - protein, 10 aa, Mus musculus
EEVQRKRQKL

SEQ ID NO: 59 - protein, 9 aa, Mus musculus
EEKRKRTYE

SEQ ID NO: 60 - protein, 20 aa, Xenopus laevis
AVKRPAATKKAGQAKKKKLD

SEQ ID NO: 61 - protein, 31 aa, Mus musculus
MASPLTRFLSLNLLLLGESILGSGEAKPQAP

SEQ ID NO: 62 - protein, 21 aa, Homo sapiens
MSSFGYRTLTVALFTLICCPG

SEQ ID NO: 63 - protein, 51 aa, Mus musculus
PQRPEDCRPRGSVKGTGLDFACDIYIWAPLAGICVALLLSLIITLICYHSR

SEQ ID NO: 64 - protein, 33 aa, Homo sapiens
MVIIVTVVSVLLSLFVTSVLLCFIFGQHLRQQR

SEQ ID NO: 65 - protein, 37 aa, Rattus sp.
PNKGSGTTSGTTRLLSGHTCFTLTGLLGTLVTMGLLT

SEQ ID NO: 66 - protein, 14 aa, Homo sapiens
MGSSKSKPKDPSQR

SEQ ID NO: 67 - protein, 26 aa, Homo sapiens
LLQRLFSRQDCCGNCSDSEEELPTRL

SEQ ID NO: 68 - protein, 20 aa, Rattus norvegicus
KQFRNCMLTSLCCGKNPLGD

SEQ ID NO: 69 - protein, 19 aa, Homo sapiens
LNPPDESGPGCMSCKCVLS

SEQ ID NO: 70 - protein, 5 aa, Artificial sequence
  lysosomal degradation sequence
KFERQ

SEQ ID NO: 71 - protein, 36 aa, Cricetulus griseus
MLIPIAGFFALAGLVLIVLIAYLIGRKRSHAGYQTI

SEQ ID NO: 72 - protein, 35 aa, Homo sapiens
LVPIAVGAALAGVLILVLLAYFIGLKHHHAGYEQF

SEQ ID NO: 73 - protein, 27 aa, Yeast
MLRTSSLFTRRVQPSLFSRNILRLQST

SEQ ID NO: 74 - protein, 25 aa, Yeast
MLSLRQSIRFFKPATRTLCSSRYLL

SEQ ID NO: 75 - protein, 64 aa, Yeast
MFSMLSKRWAQRTLSKSFYSTATGAASKSGKLTQKLVTAGVAAAGITASTLLYADSLTAEAMTA

SEQ ID NO: 76 - protein, 41 aa, Yeast
MKSFITRNKTAILATVAATGTAIGAYYYYNQLQQQQQRGKK

SEQ ID NO: 77 - protein, 4 aa, Homo sapiens
KDEL

SEQ ID NO: 78 - protein, 15 aa, unidentified adenovirus
LYLSRRSFIDEKKMP

SEQ ID NO: 79 - protein, 19 aa, Homo sapiens
LNPPDESGPGCMSCKCVLS

SEQ ID NO: 80 - protein, 15 aa, Homo sapiens
LTEPTQPTRNQCCSN

SEQ ID NO: 81 - protein, 9 aa, Unknown
  cyclin B1 destruction sequence
RTALGDIGN

SEQ ID NO: 82 - protein, 20 aa, Unknown
  signal sequence from Interleukin-2
MYRMQLLSCIALSLALVTNS

SEQ ID NO: 83 - protein, 29 aa, Homo sapiens
MATGSRTSLLLAFGLLCLPWLQEGSAFPT

SEQ ID NO: 84 - protein, 27 aa, Homo sapiens
MALWMRLLPLLALLALWGPDPAAAFVN

SEQ ID NO: 85 - protein, 18 aa, Influenza virus
MKAKLLVLLYAFVAGDQI

SEQ ID NO: 86 - protein, 24 aa, Unknown
  signal sequence from Interleukin-4
MGLTSQLLPPLFFLLACAGNFVHG

SEQ ID NO: 87 - protein, 10 aa, Artificial sequence
  stability sequence
  MISC_FEATURE (3)..(6): “Xaa” at positions 3 through 6 can be any amino acid.
MGXXXXGGPP